SEQUENCE LISTING (500 sequences)

SEQ ID NO: 1 - protein, 12 aa, Artificial Sequence
  Advanced Macromolecule Transduction Domain (aMTD) Sequences for Improvement of Cell-Permeability
AAALAPVVLALP

SEQ ID NO: 2 - protein, 12 aa, Artificial Sequence
  Advanced Macromolecule Transduction Domain (aMTD) Sequences for Improvement of Cell-Permeability
AAAVPLLAVVVP

SEQ ID NO: 3 - protein, 12 aa, Artificial Sequence
  Advanced Macromolecule Transduction Domain (aMTD) Sequences for Improvement of Cell-Permeability
AALLVPAAVLAP

SEQ ID NO: 4 - protein, 12 aa, Artificial Sequence
  Advanced Macromolecule Transduction Domain (aMTD) Sequences for Improvement of Cell-Permeability
ALALLPVAALAP

SEQ ID NO: 5 - protein, 12 aa, Artificial Sequence
  Advanced Macromolecule Transduction Domain (aMTD) Sequences for Improvement of Cell-Permeability
AAALLPVALVAP

SEQ ID NO: 6 - protein, 12 aa, Artificial Sequence
  Advanced Macromolecule Transduction Domain (aMTD) Sequences for Improvement of Cell-Permeability
VVALAPALAALP

SEQ ID NO: 7 - protein, 12 aa, Artificial Sequence
  Advanced Macromolecule Transduction Domain (aMTD) Sequences for Improvement of Cell-Permeability
LLAAVPAVLLAP

SEQ ID NO: 8 - protein, 12 aa, Artificial Sequence
  Advanced Macromolecule Transduction Domain (aMTD) Sequences for Improvement of Cell-Permeability
AAALVPVVALLP

SEQ ID NO: 9 - protein, 12 aa, Artificial Sequence
  Advanced Macromolecule Transduction Domain (aMTD) Sequences for Improvement of Cell-Permeability
AVALLPALLAVP

SEQ ID NO: 10 - protein, 12 aa, Artificial Sequence
  Advanced Macromolecule Transduction Domain (aMTD) Sequences for Improvement of Cell-Permeability
AVVLVPVLAAAP

SEQ ID NO: 11 - protein, 12 aa, Artificial Sequence
  Advanced Macromolecule Transduction Domain (aMTD) Sequences for Improvement of Cell-Permeability
VVLVLPAAAAVP

SEQ ID NO: 12 - protein, 12 aa, Artificial Sequence
  Advanced Macromolecule Transduction Domain (aMTD) Sequences for Improvement of Cell-Permeability
IALAAPALIVAP

SEQ ID NO: 13 - protein, 12 aa, Artificial Sequence
  Advanced Macromolecule Transduction Domain (aMTD) Sequences for Improvement of Cell-Permeability
IVAVAPALVALP

SEQ ID NO: 14 - protein, 12 aa, Artificial Sequence
  Advanced Macromolecule Transduction Domain (aMTD) Sequences for Improvement of Cell-Permeability
VAALPVVAVVAP

SEQ ID NO: 15 - protein, 12 aa, Artificial Sequence
  Advanced Macromolecule Transduction Domain (aMTD) Sequences for Improvement of Cell-Permeability
LLAAPLVVAAVP

SEQ ID NO: 16 - protein, 12 aa, Artificial Sequence
  Advanced Macromolecule Transduction Domain (aMTD) Sequences for Improvement of Cell-Permeability
ALAVPVALLVAP

SEQ ID NO: 17 - protein, 12 aa, Artificial Sequence
  Advanced Macromolecule Transduction Domain (aMTD) Sequences for Improvement of Cell-Permeability
VAALPVLLAALP

SEQ ID NO: 18 - protein, 12 aa, Artificial Sequence
  Advanced Macromolecule Transduction Domain (aMTD) Sequences for Improvement of Cell-Permeability
VALLAPVALAVP

SEQ ID NO: 19 - protein, 12 aa, Artificial Sequence
  Advanced Macromolecule Transduction Domain (aMTD) Sequences for Improvement of Cell-Permeability
AALLVPALVAVP

SEQ ID NO: 20 - protein, 12 aa, Artificial Sequence
  Advanced Macromolecule Transduction Domain (aMTD) Sequences for Improvement of Cell-Permeability
AIVALPVAVLAP

SEQ ID NO: 21 - protein, 12 aa, Artificial Sequence
  Advanced Macromolecule Transduction Domain (aMTD) Sequences for Improvement of Cell-Permeability
IAIVAPVVALAP

SEQ ID NO: 22 - protein, 12 aa, Artificial Sequence
  Advanced Macromolecule Transduction Domain (aMTD) Sequences for Improvement of Cell-Permeability
AALLPALAALLP

SEQ ID NO: 23 - protein, 12 aa, Artificial Sequence
  Advanced Macromolecule Transduction Domain (aMTD) Sequences for Improvement of Cell-Permeability
AVVLAPVAAVLP

SEQ ID NO: 24 - protein, 12 aa, Artificial Sequence
  Advanced Macromolecule Transduction Domain (aMTD) Sequences for Improvement of Cell-Permeability
LAVAAPLALALP

SEQ ID NO: 25 - protein, 12 aa, Artificial Sequence
  Advanced Macromolecule Transduction Domain (aMTD) Sequences for Improvement of Cell-Permeability
AAVAAPLLLALP

SEQ ID NO: 26 - protein, 12 aa, Artificial Sequence
  Advanced Macromolecule Transduction Domain (aMTD) Sequences for Improvement of Cell-Permeability
LLVLPAAALAAP

SEQ ID NO: 27 - protein, 12 aa, Artificial Sequence
  Advanced Macromolecule Transduction Domain (aMTD) Sequences for Improvement of Cell-Permeability
LVALAPVAAVLP

SEQ ID NO: 28 - protein, 12 aa, Artificial Sequence
  Advanced Macromolecule Transduction Domain (aMTD) Sequences for Improvement of Cell-Permeability
LALAPAALALLP

SEQ ID NO: 29 - protein, 12 aa, Artificial Sequence
  Advanced Macromolecule Transduction Domain (aMTD) Sequences for Improvement of Cell-Permeability
ALIAAPILALAP

SEQ ID NO: 30 - protein, 12 aa, Artificial Sequence
  Advanced Macromolecule Transduction Domain (aMTD) Sequences for Improvement of Cell-Permeability
AVVAAPLVLALP

SEQ ID NO: 31 - protein, 12 aa, Artificial Sequence
  Advanced Macromolecule Transduction Domain (aMTD) Sequences for Improvement of Cell-Permeability
LLALAPAALLAP

SEQ ID NO: 32 - protein, 12 aa, Artificial Sequence
  Advanced Macromolecule Transduction Domain (aMTD) Sequences for Improvement of Cell-Permeability
AIVALPALALAP

SEQ ID NO: 33 - protein, 12 aa, Artificial Sequence
  Advanced Macromolecule Transduction Domain (aMTD) Sequences for Improvement of Cell-Permeability
AAIIVPAALLAP

SEQ ID NO: 34 - protein, 12 aa, Artificial Sequence
  Advanced Macromolecule Transduction Domain (aMTD) Sequences for Improvement of Cell-Permeability
IAVALPALIAAP

SEQ ID NO: 35 - protein, 12 aa, Artificial Sequence
  Advanced Macromolecule Transduction Domain (aMTD) Sequences for Improvement of Cell-Permeability
AVIVLPALAVAP

SEQ ID NO: 36 - protein, 12 aa, Artificial Sequence
  Advanced Macromolecule Transduction Domain (aMTD) Sequences for Improvement of Cell-Permeability
AVLAVPAVLVAP

SEQ ID NO: 37 - protein, 12 aa, Artificial Sequence
  Advanced Macromolecule Transduction Domain (aMTD) Sequences for Improvement of Cell-Permeability
VLAIVPAVALAP

SEQ ID NO: 38 - protein, 12 aa, Artificial Sequence
  Advanced Macromolecule Transduction Domain (aMTD) Sequences for Improvement of Cell-Permeability
LLAVVPAVALAP

SEQ ID NO: 39 - protein, 12 aa, Artificial Sequence
  Advanced Macromolecule Transduction Domain (aMTD) Sequences for Improvement of Cell-Permeability
AVIALPALIAAP

SEQ ID NO: 40 - protein, 12 aa, Artificial Sequence
  Advanced Macromolecule Transduction Domain (aMTD) Sequences for Improvement of Cell-Permeability
AVVALPAALIVP

SEQ ID NO: 41 - protein, 12 aa, Artificial Sequence
  Advanced Macromolecule Transduction Domain (aMTD) Sequences for Improvement of Cell-Permeability
LALVLPAALAAP

SEQ ID NO: 42 - protein, 12 aa, Artificial Sequence
  Advanced Macromolecule Transduction Domain (aMTD) Sequences for Improvement of Cell-Permeability
LAAVLPALLAAP

SEQ ID NO: 43 - protein, 12 aa, Artificial Sequence
  Advanced Macromolecule Transduction Domain (aMTD) Sequences for Improvement of Cell-Permeability
ALAVPVALAIVP

SEQ ID NO: 44 - protein, 12 aa, Artificial Sequence
  Advanced Macromolecule Transduction Domain (aMTD) Sequences for Improvement of Cell-Permeability
ALIAPVVALVAP

SEQ ID NO: 45 - protein, 12 aa, Artificial Sequence
  Advanced Macromolecule Transduction Domain (aMTD) Sequences for Improvement of Cell-Permeability
LLAAPVVIALAP

SEQ ID NO: 46 - protein, 12 aa, Artificial Sequence
  Advanced Macromolecule Transduction Domain (aMTD) Sequences for Improvement of Cell-Permeability
LAAIVPAIIAVP

SEQ ID NO: 47 - protein, 12 aa, Artificial Sequence
  Advanced Macromolecule Transduction Domain (aMTD) Sequences for Improvement of Cell-Permeability
AALVLPLIIAAP

SEQ ID NO: 48 - protein, 12 aa, Artificial Sequence
  Advanced Macromolecule Transduction Domain (aMTD) Sequences for Improvement of Cell-Permeability
LALAVPALAALP

SEQ ID NO: 49 - protein, 12 aa, Artificial Sequence
  Advanced Macromolecule Transduction Domain (aMTD) Sequences for Improvement of Cell-Permeability
LIAALPAVAALP

SEQ ID NO: 50 - protein, 12 aa, Artificial Sequence
  Advanced Macromolecule Transduction Domain (aMTD) Sequences for Improvement of Cell-Permeability
ALALVPAIAALP

SEQ ID NO: 51 - protein, 12 aa, Artificial Sequence
  Advanced Macromolecule Transduction Domain (aMTD) Sequences for Improvement of Cell-Permeability
AAILAPIVALAP

SEQ ID NO: 52 - protein, 12 aa, Artificial Sequence
  Advanced Macromolecule Transduction Domain (aMTD) Sequences for Improvement of Cell-Permeability
ALLIAPAAVIAP

SEQ ID NO: 53 - protein, 12 aa, Artificial Sequence
  Advanced Macromolecule Transduction Domain (aMTD) Sequences for Improvement of Cell-Permeability
AILAVPIAVVAP

SEQ ID NO: 54 - protein, 12 aa, Artificial Sequence
  Advanced Macromolecule Transduction Domain (aMTD) Sequences for Improvement of Cell-Permeability
ILAAVPIALAAP

SEQ ID NO: 55 - protein, 12 aa, Artificial Sequence
  Advanced Macromolecule Transduction Domain (aMTD) Sequences for Improvement of Cell-Permeability
VAALLPAAAVLP

SEQ ID NO: 56 - protein, 12 aa, Artificial Sequence
  Advanced Macromolecule Transduction Domain (aMTD) Sequences for Improvement of Cell-Permeability
AAAVVPVLLVAP

SEQ ID NO: 57 - protein, 12 aa, Artificial Sequence
  Advanced Macromolecule Transduction Domain (aMTD) Sequences for Improvement of Cell-Permeability
AALLVPALVAAP

SEQ ID NO: 58 - protein, 12 aa, Artificial Sequence
  Advanced Macromolecule Transduction Domain (aMTD) Sequences for Improvement of Cell-Permeability
AAVLLPVALAAP

SEQ ID NO: 59 - protein, 12 aa, Artificial Sequence
  Advanced Macromolecule Transduction Domain (aMTD) Sequences for Improvement of Cell-Permeability
AAALAPVLALVP

SEQ ID NO: 60 - protein, 12 aa, Artificial Sequence
  Advanced Macromolecule Transduction Domain (aMTD) Sequences for Improvement of Cell-Permeability
LVLVPLLAAAAP

SEQ ID NO: 61 - protein, 12 aa, Artificial Sequence
  Advanced Macromolecule Transduction Domain (aMTD) Sequences for Improvement of Cell-Permeability
ALIAVPAIIVAP

SEQ ID NO: 62 - protein, 12 aa, Artificial Sequence
  Advanced Macromolecule Transduction Domain (aMTD) Sequences for Improvement of Cell-Permeability
ALAVIPAAAILP

SEQ ID NO: 63 - protein, 12 aa, Artificial Sequence
  Advanced Macromolecule Transduction Domain (aMTD) Sequences for Improvement of Cell-Permeability
LAAAPVVIVIAP

SEQ ID NO: 64 - protein, 12 aa, Artificial Sequence
  Advanced Macromolecule Transduction Domain (aMTD) Sequences for Improvement of Cell-Permeability
VLAIAPLLAAVP

SEQ ID NO: 65 - protein, 12 aa, Artificial Sequence
  Advanced Macromolecule Transduction Domain (aMTD) Sequences for Improvement of Cell-Permeability
ALIVLPAAVAVP

SEQ ID NO: 66 - protein, 12 aa, Artificial Sequence
  Advanced Macromolecule Transduction Domain (aMTD) Sequences for Improvement of Cell-Permeability
VLAVAPALIVAP

SEQ ID NO: 67 - protein, 12 aa, Artificial Sequence
  Advanced Macromolecule Transduction Domain (aMTD) Sequences for Improvement of Cell-Permeability
AALLAPALIVAP

SEQ ID NO: 68 - protein, 12 aa, Artificial Sequence
  Advanced Macromolecule Transduction Domain (aMTD) Sequences for Improvement of Cell-Permeability
ALIAPAVALIVP

SEQ ID NO: 69 - protein, 12 aa, Artificial Sequence
  Advanced Macromolecule Transduction Domain (aMTD) Sequences for Improvement of Cell-Permeability
AIVLLPAAVVAP

SEQ ID NO: 70 - protein, 12 aa, Artificial Sequence
  Advanced Macromolecule Transduction Domain (aMTD) Sequences for Improvement of Cell-Permeability
VIAAPVLAVLAP

SEQ ID NO: 71 - protein, 12 aa, Artificial Sequence
  Advanced Macromolecule Transduction Domain (aMTD) Sequences for Improvement of Cell-Permeability
LALAPALALLAP

SEQ ID NO: 72 - protein, 12 aa, Artificial Sequence
  Advanced Macromolecule Transduction Domain (aMTD) Sequences for Improvement of Cell-Permeability
AIILAPIAAIAP

SEQ ID NO: 73 - protein, 12 aa, Artificial Sequence
  Advanced Macromolecule Transduction Domain (aMTD) Sequences for Improvement of Cell-Permeability
IALAAPILLAAP

SEQ ID NO: 74 - protein, 12 aa, Artificial Sequence
  Advanced Macromolecule Transduction Domain (aMTD) Sequences for Improvement of Cell-Permeability
IVAVALPALAVP

SEQ ID NO: 75 - protein, 12 aa, Artificial Sequence
  Advanced Macromolecule Transduction Domain (aMTD) Sequences for Improvement of Cell-Permeability
VVAIVLPALAAP

SEQ ID NO: 76 - protein, 12 aa, Artificial Sequence
  Advanced Macromolecule Transduction Domain (aMTD) Sequences for Improvement of Cell-Permeability
IVAVALPVALAP

SEQ ID NO: 77 - protein, 12 aa, Artificial Sequence
  Advanced Macromolecule Transduction Domain (aMTD) Sequences for Improvement of Cell-Permeability
IVAVALPAALVP

SEQ ID NO: 78 - protein, 12 aa, Artificial Sequence
  Advanced Macromolecule Transduction Domain (aMTD) Sequences for Improvement of Cell-Permeability
IVAVALPAVALP

SEQ ID NO: 79 - protein, 12 aa, Artificial Sequence
  Advanced Macromolecule Transduction Domain (aMTD) Sequences for Improvement of Cell-Permeability
IVAVALPAVLAP

SEQ ID NO: 80 - protein, 12 aa, Artificial Sequence
  Advanced Macromolecule Transduction Domain (aMTD) Sequences for Improvement of Cell-Permeability
VIVALAPAVLAP

SEQ ID NO: 81 - protein, 12 aa, Artificial Sequence
  Advanced Macromolecule Transduction Domain (aMTD) Sequences for Improvement of Cell-Permeability
IVAVALPALVAP

SEQ ID NO: 82 - protein, 12 aa, Artificial Sequence
  Advanced Macromolecule Transduction Domain (aMTD) Sequences for Improvement of Cell-Permeability
ALLIVAPVAVAP

SEQ ID NO: 83 - protein, 12 aa, Artificial Sequence
  Advanced Macromolecule Transduction Domain (aMTD) Sequences for Improvement of Cell-Permeability
AVVIVAPAVIAP

SEQ ID NO: 84 - protein, 12 aa, Artificial Sequence
  Advanced Macromolecule Transduction Domain (aMTD) Sequences for Improvement of Cell-Permeability
AVLAVAPALIVP

SEQ ID NO: 85 - protein, 12 aa, Artificial Sequence
  Advanced Macromolecule Transduction Domain (aMTD) Sequences for Improvement of Cell-Permeability
LVAAVAPALIVP

SEQ ID NO: 86 - protein, 12 aa, Artificial Sequence
  Advanced Macromolecule Transduction Domain (aMTD) Sequences for Improvement of Cell-Permeability
AVIVVAPALLAP

SEQ ID NO: 87 - protein, 12 aa, Artificial Sequence
  Advanced Macromolecule Transduction Domain (aMTD) Sequences for Improvement of Cell-Permeability
VVAIVLPAVAAP

SEQ ID NO: 88 - protein, 12 aa, Artificial Sequence
  Advanced Macromolecule Transduction Domain (aMTD) Sequences for Improvement of Cell-Permeability
AAALVIPAILAP

SEQ ID NO: 89 - protein, 12 aa, Artificial Sequence
  Advanced Macromolecule Transduction Domain (aMTD) Sequences for Improvement of Cell-Permeability
VIVALAPALLAP

SEQ ID NO: 90 - protein, 12 aa, Artificial Sequence
  Advanced Macromolecule Transduction Domain (aMTD) Sequences for Improvement of Cell-Permeability
VIVAIAPALLAP

SEQ ID NO: 91 - protein, 12 aa, Artificial Sequence
  Advanced Macromolecule Transduction Domain (aMTD) Sequences for Improvement of Cell-Permeability
IVAIAVPALVAP

SEQ ID NO: 92 - protein, 12 aa, Artificial Sequence
  Advanced Macromolecule Transduction Domain (aMTD) Sequences for Improvement of Cell-Permeability
AALAVIPAAILP

SEQ ID NO: 93 - protein, 12 aa, Artificial Sequence
  Advanced Macromolecule Transduction Domain (aMTD) Sequences for Improvement of Cell-Permeability
ALAAVIPAAILP

SEQ ID NO: 94 - protein, 12 aa, Artificial Sequence
  Advanced Macromolecule Transduction Domain (aMTD) Sequences for Improvement of Cell-Permeability
AAALVIPAAILP

SEQ ID NO: 95 - protein, 12 aa, Artificial Sequence
  Advanced Macromolecule Transduction Domain (aMTD) Sequences for Improvement of Cell-Permeability
LAAAVIPAAILP

SEQ ID NO: 96 - protein, 12 aa, Artificial Sequence
  Advanced Macromolecule Transduction Domain (aMTD) Sequences for Improvement of Cell-Permeability
LAAAVIPVAILP

SEQ ID NO: 97 - protein, 12 aa, Artificial Sequence
  Advanced Macromolecule Transduction Domain (aMTD) Sequences for Improvement of Cell-Permeability
AAILAAPLIAVP

SEQ ID NO: 98 - protein, 12 aa, Artificial Sequence
  Advanced Macromolecule Transduction Domain (aMTD) Sequences for Improvement of Cell-Permeability
VVAILAPLLAAP

SEQ ID NO: 99 - protein, 12 aa, Artificial Sequence
  Advanced Macromolecule Transduction Domain (aMTD) Sequences for Improvement of Cell-Permeability
AVVVAAPVLALP

SEQ ID NO: 100 - protein, 12 aa, Artificial Sequence
  Advanced Macromolecule Transduction Domain (aMTD) Sequences for Improvement of Cell-Permeability
AVVAIAPVLALP

SEQ ID NO: 101 - protein, 12 aa, Artificial Sequence
  Advanced Macromolecule Transduction Domain (aMTD) Sequences for Improvement of Cell-Permeability
ALAALVPAVLVP

SEQ ID NO: 102 - protein, 12 aa, Artificial Sequence
  Advanced Macromolecule Transduction Domain (aMTD) Sequences for Improvement of Cell-Permeability
ALAALVPVALVP

SEQ ID NO: 103 - protein, 12 aa, Artificial Sequence
  Advanced Macromolecule Transduction Domain (aMTD) Sequences for Improvement of Cell-Permeability
LAAALVPVALVP

SEQ ID NO: 104 - protein, 12 aa, Artificial Sequence
  Advanced Macromolecule Transduction Domain (aMTD) Sequences for Improvement of Cell-Permeability
ALAALVPALVVP

SEQ ID NO: 105 - protein, 12 aa, Artificial Sequence
  Advanced Macromolecule Transduction Domain (aMTD) Sequences for Improvement of Cell-Permeability
IAAVIVPAVALP

SEQ ID NO: 106 - protein, 12 aa, Artificial Sequence
  Advanced Macromolecule Transduction Domain (aMTD) Sequences for Improvement of Cell-Permeability
IAAVLVPAVALP

SEQ ID NO: 107 - protein, 12 aa, Artificial Sequence
  Advanced Macromolecule Transduction Domain (aMTD) Sequences for Improvement of Cell-Permeability
AVAILVPLLAAP

SEQ ID NO: 108 - protein, 12 aa, Artificial Sequence
  Advanced Macromolecule Transduction Domain (aMTD) Sequences for Improvement of Cell-Permeability
AVVILVPLAAAP

SEQ ID NO: 109 - protein, 12 aa, Artificial Sequence
  Advanced Macromolecule Transduction Domain (aMTD) Sequences for Improvement of Cell-Permeability
IAAVIVPVAALP

SEQ ID NO: 110 - protein, 12 aa, Artificial Sequence
  Advanced Macromolecule Transduction Domain (aMTD) Sequences for Improvement of Cell-Permeability
AIAIAIVPVALP

SEQ ID NO: 111 - protein, 12 aa, Artificial Sequence
  Advanced Macromolecule Transduction Domain (aMTD) Sequences for Improvement of Cell-Permeability
ILAVAAIPVAVP

SEQ ID NO: 112 - protein, 12 aa, Artificial Sequence
  Advanced Macromolecule Transduction Domain (aMTD) Sequences for Improvement of Cell-Permeability
ILAAAIIPAALP

SEQ ID NO: 113 - protein, 12 aa, Artificial Sequence
  Advanced Macromolecule Transduction Domain (aMTD) Sequences for Improvement of Cell-Permeability
LAVVLAAPAIVP

SEQ ID NO: 114 - protein, 12 aa, Artificial Sequence
  Advanced Macromolecule Transduction Domain (aMTD) Sequences for Improvement of Cell-Permeability
AILAAIVPLAVP

SEQ ID NO: 115 - protein, 12 aa, Artificial Sequence
  Advanced Macromolecule Transduction Domain (aMTD) Sequences for Improvement of Cell-Permeability
VIVALAVPALAP

SEQ ID NO: 116 - protein, 12 aa, Artificial Sequence
  Advanced Macromolecule Transduction Domain (aMTD) Sequences for Improvement of Cell-Permeability
AIVALAVPVLAP

SEQ ID NO: 117 - protein, 12 aa, Artificial Sequence
  Advanced Macromolecule Transduction Domain (aMTD) Sequences for Improvement of Cell-Permeability
AAIIIVLPAALP

SEQ ID NO: 118 - protein, 12 aa, Artificial Sequence
  Advanced Macromolecule Transduction Domain (aMTD) Sequences for Improvement of Cell-Permeability
LIVALAVPALAP

SEQ ID NO: 119 - protein, 12 aa, Artificial Sequence
  Advanced Macromolecule Transduction Domain (aMTD) Sequences for Improvement of Cell-Permeability
AIIIVIAPAAAP

SEQ ID NO: 120 - protein, 12 aa, Artificial Sequence
  Advanced Macromolecule Transduction Domain (aMTD) Sequences for Improvement of Cell-Permeability
LAALIVVPAVAP

SEQ ID NO: 121 - protein, 12 aa, Artificial Sequence
  Advanced Macromolecule Transduction Domain (aMTD) Sequences for Improvement of Cell-Permeability
ALLVIAVPAVAP

SEQ ID NO: 122 - protein, 12 aa, Artificial Sequence
  Advanced Macromolecule Transduction Domain (aMTD) Sequences for Improvement of Cell-Permeability
AVALIVVPALAP

SEQ ID NO: 123 - protein, 12 aa, Artificial Sequence
  Advanced Macromolecule Transduction Domain (aMTD) Sequences for Improvement of Cell-Permeability
ALAIVVAPVAVP

SEQ ID NO: 124 - protein, 12 aa, Artificial Sequence
  Advanced Macromolecule Transduction Domain (aMTD) Sequences for Improvement of Cell-Permeability
LLALIIAPAAAP

SEQ ID NO: 125 - protein, 12 aa, Artificial Sequence
  Advanced Macromolecule Transduction Domain (aMTD) Sequences for Improvement of Cell-Permeability
ALALIIVPAVAP

SEQ ID NO: 126 - protein, 12 aa, Artificial Sequence
  Advanced Macromolecule Transduction Domain (aMTD) Sequences for Improvement of Cell-Permeability
LLAALIAPAALP

SEQ ID NO: 127 - protein, 12 aa, Artificial Sequence
  Advanced Macromolecule Transduction Domain (aMTD) Sequences for Improvement of Cell-Permeability
IVALIVAPAAVP

SEQ ID NO: 128 - protein, 12 aa, Artificial Sequence
  Advanced Macromolecule Transduction Domain (aMTD) Sequences for Improvement of Cell-Permeability
VVLVLAAPAAVP

SEQ ID NO: 129 - protein, 12 aa, Artificial Sequence
  Advanced Macromolecule Transduction Domain (aMTD) Sequences for Improvement of Cell-Permeability
AAVAIVLPAVVP

SEQ ID NO: 130 - protein, 12 aa, Artificial Sequence
  Advanced Macromolecule Transduction Domain (aMTD) Sequences for Improvement of Cell-Permeability
ALIAAIVPALVP

SEQ ID NO: 131 - protein, 12 aa, Artificial Sequence
  Advanced Macromolecule Transduction Domain (aMTD) Sequences for Improvement of Cell-Permeability
ALAVIVVPALAP

SEQ ID NO: 132 - protein, 12 aa, Artificial Sequence
  Advanced Macromolecule Transduction Domain (aMTD) Sequences for Improvement of Cell-Permeability
VAIALIVPALAP

SEQ ID NO: 133 - protein, 12 aa, Artificial Sequence
  Advanced Macromolecule Transduction Domain (aMTD) Sequences for Improvement of Cell-Permeability
VAIVLVAPAVAP

SEQ ID NO: 134 - protein, 12 aa, Artificial Sequence
  Advanced Macromolecule Transduction Domain (aMTD) Sequences for Improvement of Cell-Permeability
VAVALIVPALAP

SEQ ID NO: 135 - protein, 12 aa, Artificial Sequence
  Advanced Macromolecule Transduction Domain (aMTD) Sequences for Improvement of Cell-Permeability
AVILALAPIVAP

SEQ ID NO: 136 - protein, 12 aa, Artificial Sequence
  Advanced Macromolecule Transduction Domain (aMTD) Sequences for Improvement of Cell-Permeability
ALIVAIAPALVP

SEQ ID NO: 137 - protein, 12 aa, Artificial Sequence
  Advanced Macromolecule Transduction Domain (aMTD) Sequences for Improvement of Cell-Permeability
AAILIAVPIAAP

SEQ ID NO: 138 - protein, 12 aa, Artificial Sequence
  Advanced Macromolecule Transduction Domain (aMTD) Sequences for Improvement of Cell-Permeability
VIVALAAPVLAP

SEQ ID NO: 139 - protein, 12 aa, Artificial Sequence
  Advanced Macromolecule Transduction Domain (aMTD) Sequences for Improvement of Cell-Permeability
VLVALAAPVIAP

SEQ ID NO: 140 - protein, 12 aa, Artificial Sequence
  Advanced Macromolecule Transduction Domain (aMTD) Sequences for Improvement of Cell-Permeability
VALIAVAPAVVP

SEQ ID NO: 141 - protein, 12 aa, Artificial Sequence
  Advanced Macromolecule Transduction Domain (aMTD) Sequences for Improvement of Cell-Permeability
VIAAVLAPVAVP

SEQ ID NO: 142 - protein, 12 aa, Artificial Sequence
  Advanced Macromolecule Transduction Domain (aMTD) Sequences for Improvement of Cell-Permeability
ALIVLAAPVAVP

SEQ ID NO: 143 - protein, 12 aa, Artificial Sequence
  Advanced Macromolecule Transduction Domain (aMTD) Sequences for Improvement of Cell-Permeability
VAAAIALPAIVP

SEQ ID NO: 144 - protein, 12 aa, Artificial Sequence
  Advanced Macromolecule Transduction Domain (aMTD) Sequences for Improvement of Cell-Permeability
ILAAAAAPLIVP

SEQ ID NO: 145 - protein, 12 aa, Artificial Sequence
  Advanced Macromolecule Transduction Domain (aMTD) Sequences for Improvement of Cell-Permeability
LALVLAAPAIVP

SEQ ID NO: 146 - protein, 12 aa, Artificial Sequence
  Advanced Macromolecule Transduction Domain (aMTD) Sequences for Improvement of Cell-Permeability
ALAVVALPAIVP

SEQ ID NO: 147 - protein, 12 aa, Artificial Sequence
  Advanced Macromolecule Transduction Domain (aMTD) Sequences for Improvement of Cell-Permeability
AAILAPIVAALP

SEQ ID NO: 148 - protein, 12 aa, Artificial Sequence
  Advanced Macromolecule Transduction Domain (aMTD) Sequences for Improvement of Cell-Permeability
ILIAIAIPAAAP

SEQ ID NO: 149 - protein, 12 aa, Artificial Sequence
  Advanced Macromolecule Transduction Domain (aMTD) Sequences for Improvement of Cell-Permeability
LAIVLAAPVAVP

SEQ ID NO: 150 - protein, 12 aa, Artificial Sequence
  Advanced Macromolecule Transduction Domain (aMTD) Sequences for Improvement of Cell-Permeability
AAIAIIAPAIVP

SEQ ID NO: 151 - protein, 12 aa, Artificial Sequence
  Advanced Macromolecule Transduction Domain (aMTD) Sequences for Improvement of Cell-Permeability
LAVAIVAPALVP

SEQ ID NO: 152 - protein, 12 aa, Artificial Sequence
  Advanced Macromolecule Transduction Domain (aMTD) Sequences for Improvement of Cell-Permeability
LAIVLAAPAVLP

SEQ ID NO: 153 - protein, 12 aa, Artificial Sequence
  Advanced Macromolecule Transduction Domain (aMTD) Sequences for Improvement of Cell-Permeability
AAIVLALPAVLP

SEQ ID NO: 154 - protein, 12 aa, Artificial Sequence
  Advanced Macromolecule Transduction Domain (aMTD) Sequences for Improvement of Cell-Permeability
ALLVAVLPAALP

SEQ ID NO: 155 - protein, 12 aa, Artificial Sequence
  Advanced Macromolecule Transduction Domain (aMTD) Sequences for Improvement of Cell-Permeability
AALVAVLPVALP

SEQ ID NO: 156 - protein, 12 aa, Artificial Sequence
  Advanced Macromolecule Transduction Domain (aMTD) Sequences for Improvement of Cell-Permeability
AILAVALPLLAP

SEQ ID NO: 157 - protein, 12 aa, Artificial Sequence
  Advanced Macromolecule Transduction Domain (aMTD) Sequences for Improvement of Cell-Permeability
IVAVALVPALAP

SEQ ID NO: 158 - protein, 12 aa, Artificial Sequence
  Advanced Macromolecule Transduction Domain (aMTD) Sequences for Improvement of Cell-Permeability
IVAVALLPALAP

SEQ ID NO: 159 - protein, 12 aa, Artificial Sequence
  Advanced Macromolecule Transduction Domain (aMTD) Sequences for Improvement of Cell-Permeability
IVAVALLPAVAP

SEQ ID NO: 160 - protein, 12 aa, Artificial Sequence
  Advanced Macromolecule Transduction Domain (aMTD) Sequences for Improvement of Cell-Permeability
IVALAVLPAVAP

SEQ ID NO: 161 - protein, 12 aa, Artificial Sequence
  Advanced Macromolecule Transduction Domain (aMTD) Sequences for Improvement of Cell-Permeability
VAVLAVLPALAP

SEQ ID NO: 162 - protein, 12 aa, Artificial Sequence
  Advanced Macromolecule Transduction Domain (aMTD) Sequences for Improvement of Cell-Permeability
IAVLAVAPAVLP

SEQ ID NO: 163 - protein, 12 aa, Artificial Sequence
  Advanced Macromolecule Transduction Domain (aMTD) Sequences for Improvement of Cell-Permeability
LAVAIIAPAVAP

SEQ ID NO: 164 - protein, 12 aa, Artificial Sequence
  Advanced Macromolecule Transduction Domain (aMTD) Sequences for Improvement of Cell-Permeability
VALAIALPAVLP

SEQ ID NO: 165 - protein, 12 aa, Artificial Sequence
  Advanced Macromolecule Transduction Domain (aMTD) Sequences for Improvement of Cell-Permeability
AIAIALVPVALP

SEQ ID NO: 166 - protein, 12 aa, Artificial Sequence
  Advanced Macromolecule Transduction Domain (aMTD) Sequences for Improvement of Cell-Permeability
AAVVIVAPVALP

SEQ ID NO: 167 - protein, 12 aa, Artificial Sequence
  Advanced Macromolecule Transduction Domain (aMTD) Sequences for Improvement of Cell-Permeability
VAIIVVAPALAP

SEQ ID NO: 168 - protein, 12 aa, Artificial Sequence
  Advanced Macromolecule Transduction Domain (aMTD) Sequences for Improvement of Cell-Permeability
VALLAIAPALAP

SEQ ID NO: 169 - protein, 12 aa, Artificial Sequence
  Advanced Macromolecule Transduction Domain (aMTD) Sequences for Improvement of Cell-Permeability
VAVLIAVPALAP

SEQ ID NO: 170 - protein, 12 aa, Artificial Sequence
  Advanced Macromolecule Transduction Domain (aMTD) Sequences for Improvement of Cell-Permeability
AVALAVLPAVVP

SEQ ID NO: 171 - protein, 12 aa, Artificial Sequence
  Advanced Macromolecule Transduction Domain (aMTD) Sequences for Improvement of Cell-Permeability
AVALAVVPAVLP

SEQ ID NO: 172 - protein, 12 aa, Artificial Sequence
  Advanced Macromolecule Transduction Domain (aMTD) Sequences for Improvement of Cell-Permeability
IVVIAVAPAVAP

SEQ ID NO: 173 - protein, 12 aa, Artificial Sequence
  Advanced Macromolecule Transduction Domain (aMTD) Sequences for Improvement of Cell-Permeability
IVVAAVVPALAP

SEQ ID NO: 174 - protein, 12 aa, Artificial Sequence
  Advanced Macromolecule Transduction Domain (aMTD) Sequences for Improvement of Cell-Permeability
IVALVPAVAIAP

SEQ ID NO: 175 - protein, 12 aa, Artificial Sequence
  Advanced Macromolecule Transduction Domain (aMTD) Sequences for Improvement of Cell-Permeability
VAALPAVALVVP

SEQ ID NO: 176 - protein, 12 aa, Artificial Sequence
  Advanced Macromolecule Transduction Domain (aMTD) Sequences for Improvement of Cell-Permeability
LVAIAPLAVLAP

SEQ ID NO: 177 - protein, 12 aa, Artificial Sequence
  Advanced Macromolecule Transduction Domain (aMTD) Sequences for Improvement of Cell-Permeability
AVALVPVIVAAP

SEQ ID NO: 178 - protein, 12 aa, Artificial Sequence
  Advanced Macromolecule Transduction Domain (aMTD) Sequences for Improvement of Cell-Permeability
AIAVAIAPVALP

SEQ ID NO: 179 - protein, 12 aa, Artificial Sequence
  Advanced Macromolecule Transduction Domain (aMTD) Sequences for Improvement of Cell-Permeability
AIALAVPVLALP

SEQ ID NO: 180 - protein, 12 aa, Artificial Sequence
  Advanced Macromolecule Transduction Domain (aMTD) Sequences for Improvement of Cell-Permeability
LVLIAAAPIALP

SEQ ID NO: 181 - protein, 12 aa, Artificial Sequence
  Advanced Macromolecule Transduction Domain (aMTD) Sequences for Improvement of Cell-Permeability
LVALAVPAAVLP

SEQ ID NO: 182 - protein, 12 aa, Artificial Sequence
  Advanced Macromolecule Transduction Domain (aMTD) Sequences for Improvement of Cell-Permeability
AVALAVPALVLP

SEQ ID NO: 183 - protein, 12 aa, Artificial Sequence
  Advanced Macromolecule Transduction Domain (aMTD) Sequences for Improvement of Cell-Permeability
LVVLAAAPLAVP

SEQ ID NO: 184 - protein, 12 aa, Artificial Sequence
  Advanced Macromolecule Transduction Domain (aMTD) Sequences for Improvement of Cell-Permeability
LIVLAAPALAAP

SEQ ID NO: 185 - protein, 12 aa, Artificial Sequence
  Advanced Macromolecule Transduction Domain (aMTD) Sequences for Improvement of Cell-Permeability
VIVLAAPALAAP

SEQ ID NO: 186 - protein, 12 aa, Artificial Sequence
  Advanced Macromolecule Transduction Domain (aMTD) Sequences for Improvement of Cell-Permeability
AVVLAVPALAVP

SEQ ID NO: 187 - protein, 12 aa, Artificial Sequence
  Advanced Macromolecule Transduction Domain (aMTD) Sequences for Improvement of Cell-Permeability
LIIVAAAPAVAP

SEQ ID NO: 188 - protein, 12 aa, Artificial Sequence
  Advanced Macromolecule Transduction Domain (aMTD) Sequences for Improvement of Cell-Permeability
IVAVIVAPAVAP

SEQ ID NO: 189 - protein, 12 aa, Artificial Sequence
  Advanced Macromolecule Transduction Domain (aMTD) Sequences for Improvement of Cell-Permeability
LVALAAPIIAVP

SEQ ID NO: 190 - protein, 12 aa, Artificial Sequence
  Advanced Macromolecule Transduction Domain (aMTD) Sequences for Improvement of Cell-Permeability
IAAVLAAPALVP

SEQ ID NO: 191 - protein, 12 aa, Artificial Sequence
  Advanced Macromolecule Transduction Domain (aMTD) Sequences for Improvement of Cell-Permeability
IALLAAPIIAVP

SEQ ID NO: 192 - protein, 12 aa, Artificial Sequence
  Advanced Macromolecule Transduction Domain (aMTD) Sequences for Improvement of Cell-Permeability
AALALVAPVIVP

SEQ ID NO: 193 - protein, 12 aa, Artificial Sequence
  Advanced Macromolecule Transduction Domain (aMTD) Sequences for Improvement of Cell-Permeability
IALVAAPVALVP

SEQ ID NO: 194 - protein, 12 aa, Artificial Sequence
  Advanced Macromolecule Transduction Domain (aMTD) Sequences for Improvement of Cell-Permeability
IIVAVAPAAIVP

SEQ ID NO: 195 - protein, 12 aa, Artificial Sequence
  Advanced Macromolecule Transduction Domain (aMTD) Sequences for Improvement of Cell-Permeability
AVAAIVPVIVAP

SEQ ID NO: 196 - protein, 12 aa, Artificial Sequence
  Advanced Macromolecule Transduction Domain (aMTD) Sequences for Improvement of Cell-Permeability
AVLVLVAPAAAP

SEQ ID NO: 197 - protein, 12 aa, Artificial Sequence
  Advanced Macromolecule Transduction Domain (aMTD) Sequences for Improvement of Cell-Permeability
VVALLAPLIAAP

SEQ ID NO: 198 - protein, 12 aa, Artificial Sequence
  Advanced Macromolecule Transduction Domain (aMTD) Sequences for Improvement of Cell-Permeability
AAVVIAPLLAVP

SEQ ID NO: 199 - protein, 12 aa, Artificial Sequence
  Advanced Macromolecule Transduction Domain (aMTD) Sequences for Improvement of Cell-Permeability
IAVAVAAPLLVP

SEQ ID NO: 200 - protein, 12 aa, Artificial Sequence
  Advanced Macromolecule Transduction Domain (aMTD) Sequences for Improvement of Cell-Permeability
LVAIVVLPAVAP

SEQ ID NO: 201 - protein, 12 aa, Artificial Sequence
  Advanced Macromolecule Transduction Domain (aMTD) Sequences for Improvement of Cell-Permeability
AVAIVVLPAVAP

SEQ ID NO: 202 - protein, 12 aa, Artificial Sequence
  Advanced Macromolecule Transduction Domain (aMTD) Sequences for Improvement of Cell-Permeability
AVILLAPLIAAP

SEQ ID NO: 203 - protein, 12 aa, Artificial Sequence
  Advanced Macromolecule Transduction Domain (aMTD) Sequences for Improvement of Cell-Permeability
LVIALAAPVALP

SEQ ID NO: 204 - protein, 12 aa, Artificial Sequence
  Advanced Macromolecule Transduction Domain (aMTD) Sequences for Improvement of Cell-Permeability
VLAVVLPAVALP

SEQ ID NO: 205 - protein, 12 aa, Artificial Sequence
  Advanced Macromolecule Transduction Domain (aMTD) Sequences for Improvement of Cell-Permeability
VLAVAAPAVLLP

SEQ ID NO: 206 - protein, 12 aa, Artificial Sequence
  Advanced Macromolecule Transduction Domain (aMTD) Sequences for Improvement of Cell-Permeability
AAVVLLPIIAAP

SEQ ID NO: 207 - protein, 12 aa, Artificial Sequence
  Advanced Macromolecule Transduction Domain (aMTD) Sequences for Improvement of Cell-Permeability
ALLVIAPAIAVP

SEQ ID NO: 208 - protein, 12 aa, Artificial Sequence
  Advanced Macromolecule Transduction Domain (aMTD) Sequences for Improvement of Cell-Permeability
AVLVIAVPAIAP

SEQ ID NO: 209 - protein, 12 aa, Artificial Sequence
  Advanced Macromolecule Transduction Domain (aMTD) Sequences for Improvement of Cell-Permeability
ALLVVIAPLAAP

SEQ ID NO: 210 - protein, 12 aa, Artificial Sequence
  Advanced Macromolecule Transduction Domain (aMTD) Sequences for Improvement of Cell-Permeability
VLVAAILPAAIP

SEQ ID NO: 211 - protein, 12 aa, Artificial Sequence
  Advanced Macromolecule Transduction Domain (aMTD) Sequences for Improvement of Cell-Permeability
VLVAAVLPIAAP

SEQ ID NO: 212 - protein, 12 aa, Artificial Sequence
  Advanced Macromolecule Transduction Domain (aMTD) Sequences for Improvement of Cell-Permeability
VLAAAVLPLVVP

SEQ ID NO: 213 - protein, 12 aa, Artificial Sequence
  Advanced Macromolecule Transduction Domain (aMTD) Sequences for Improvement of Cell-Permeability
AIAIVVPAVAVP

SEQ ID NO: 214 - protein, 12 aa, Artificial Sequence
  Advanced Macromolecule Transduction Domain (aMTD) Sequences for Improvement of Cell-Permeability
VAIIAVPAVVAP

SEQ ID NO: 215 - protein, 12 aa, Artificial Sequence
  Advanced Macromolecule Transduction Domain (aMTD) Sequences for Improvement of Cell-Permeability
IVALVAPAAVVP

SEQ ID NO: 216 - protein, 12 aa, Artificial Sequence
  Advanced Macromolecule Transduction Domain (aMTD) Sequences for Improvement of Cell-Permeability
AAIVLLPAVVVP

SEQ ID NO: 217 - protein, 12 aa, Artificial Sequence
  Advanced Macromolecule Transduction Domain (aMTD) Sequences for Improvement of Cell-Permeability
AALIVVPAVAVP

SEQ ID NO: 218 - protein, 12 aa, Artificial Sequence
  Advanced Macromolecule Transduction Domain (aMTD) Sequences for Improvement of Cell-Permeability
AIALVVPAVAVP

SEQ ID NO: 219 - protein, 12 aa, Artificial Sequence
  Advanced Macromolecule Transduction Domain (aMTD) Sequences for Improvement of Cell-Permeability
LAIVPAAIAALP

SEQ ID NO: 220 - protein, 12 aa, Artificial Sequence
  Advanced Macromolecule Transduction Domain (aMTD) Sequences for Improvement of Cell-Permeability
LVAIAPAVAVLP

SEQ ID NO: 221 - protein, 12 aa, Artificial Sequence
  Advanced Macromolecule Transduction Domain (aMTD) Sequences for Improvement of Cell-Permeability
VLAVAPAVAVLP

SEQ ID NO: 222 - protein, 12 aa, Artificial Sequence
  Advanced Macromolecule Transduction Domain (aMTD) Sequences for Improvement of Cell-Permeability
ILAVVAIPAAAP

SEQ ID NO: 223 - protein, 12 aa, Artificial Sequence
  Advanced Macromolecule Transduction Domain (aMTD) Sequences for Improvement of Cell-Permeability
ILVAAAPIAALP

SEQ ID NO: 224 - protein, 12 aa, Artificial Sequence
  Advanced Macromolecule Transduction Domain (aMTD) Sequences for Improvement of Cell-Permeability
ILAVAAIPAALP

SEQ ID NO: 225 - protein, 12 aa, Artificial Sequence
  Advanced Macromolecule Transduction Domain (aMTD) Sequences for Improvement of Cell-Permeability
VIAIPAILAAAP

SEQ ID NO: 226 - protein, 12 aa, Artificial Sequence
  Advanced Macromolecule Transduction Domain (aMTD) Sequences for Improvement of Cell-Permeability
AIIIVVPAIAAP

SEQ ID NO: 227 - protein, 12 aa, Artificial Sequence
  Advanced Macromolecule Transduction Domain (aMTD) Sequences for Improvement of Cell-Permeability
AILIVVAPIAAP

SEQ ID NO: 228 - protein, 12 aa, Artificial Sequence
  Advanced Macromolecule Transduction Domain (aMTD) Sequences for Improvement of Cell-Permeability
AVIVPVAIIAAP

SEQ ID NO: 229 - protein, 12 aa, Artificial Sequence
  Advanced Macromolecule Transduction Domain (aMTD) Sequences for Improvement of Cell-Permeability
AVVIALPAVVAP

SEQ ID NO: 230 - protein, 12 aa, Artificial Sequence
  Advanced Macromolecule Transduction Domain (aMTD) Sequences for Improvement of Cell-Permeability
ALVAVIAPVVAP

SEQ ID NO: 231 - protein, 12 aa, Artificial Sequence
  Advanced Macromolecule Transduction Domain (aMTD) Sequences for Improvement of Cell-Permeability
ALVAVLPAVAVP

SEQ ID NO: 232 - protein, 12 aa, Artificial Sequence
  Advanced Macromolecule Transduction Domain (aMTD) Sequences for Improvement of Cell-Permeability
ALVAPLLAVAVP

SEQ ID NO: 233 - protein, 12 aa, Artificial Sequence
  Advanced Macromolecule Transduction Domain (aMTD) Sequences for Improvement of Cell-Permeability
AVLAVVAPVVAP

SEQ ID NO: 234 - protein, 12 aa, Artificial Sequence
  Advanced Macromolecule Transduction Domain (aMTD) Sequences for Improvement of Cell-Permeability
AVIAVAPLVVAP

SEQ ID NO: 235 - protein, 12 aa, Artificial Sequence
  Advanced Macromolecule Transduction Domain (aMTD) Sequences for Improvement of Cell-Permeability
AVIALAPVVVAP

SEQ ID NO: 236 - protein, 12 aa, Artificial Sequence
  Advanced Macromolecule Transduction Domain (aMTD) Sequences for Improvement of Cell-Permeability
VAIALAPVVVAP

SEQ ID NO: 237 - protein, 12 aa, Artificial Sequence
  Advanced Macromolecule Transduction Domain (aMTD) Sequences for Improvement of Cell-Permeability
VALALAPVVVAP

SEQ ID NO: 238 - protein, 12 aa, Artificial Sequence
  Advanced Macromolecule Transduction Domain (aMTD) Sequences for Improvement of Cell-Permeability
VAALLPAVVVAP

SEQ ID NO: 239 - protein, 12 aa, Artificial Sequence
  Advanced Macromolecule Transduction Domain (aMTD) Sequences for Improvement of Cell-Permeability
VALALPAVVVAP

SEQ ID NO: 240 - protein, 12 aa, Artificial Sequence
  Advanced Macromolecule Transduction Domain (aMTD) Sequences for Improvement of Cell-Permeability
VALLAPAVVVAP

SEQ ID NO: 241 - DNA, 36 bp, Artificial Sequence
  The polynucleotide sequence that encodes advanced Macromolecule Transduction Domain (aMTD) peptide for Improvement of Cell-Permeability
gcggcggcgctggcgccggtggtgctggcgctgccg

SEQ ID NO: 242 - DNA, 36 bp, Artificial Sequence
  The polynucleotide sequence that encodes advanced Macromolecule Transduction Domain (aMTD) peptide for Improvement of Cell-Permeability
gcggcggcggtgccgctgctggcggtggtggtgccg

SEQ ID NO: 243 - DNA, 36 bp, Artificial Sequence
  The polynucleotide sequence that encodes advanced Macromolecule Transduction Domain (aMTD) peptide for Improvement of Cell-Permeability
gcggcgctgctggtgccggcggcggtgctggcgccg

SEQ ID NO: 244 - DNA, 36 bp, Artificial Sequence
  The polynucleotide sequence that encodes advanced Macromolecule Transduction Domain (aMTD) peptide for Improvement of Cell-Permeability
gcgctggcgctgctgccggtggcggcgctggcgccg

SEQ ID NO: 245 - DNA, 36 bp, Artificial Sequence
  The polynucleotide sequence that encodes advanced Macromolecule Transduction Domain (aMTD) peptide for Improvement of Cell-Permeability
gcggcggcgctgctgccggtggcgctggtggcgccg

SEQ ID NO: 246 - DNA, 36 bp, Artificial Sequence
  The polynucleotide sequence that encodes advanced Macromolecule Transduction Domain (aMTD) peptide for Improvement of Cell-Permeability
gtggtggcgctggcgccggcgctggcggcgctgccg

SEQ ID NO: 247 - DNA, 36 bp, Artificial Sequence
  The polynucleotide sequence that encodes advanced Macromolecule Transduction Domain (aMTD) peptide for Improvement of Cell-Permeability
ctgctggcggcggtgccggcggtgctgctggcgccg

SEQ ID NO: 248 - DNA, 36 bp, Artificial Sequence
  The polynucleotide sequence that encodes advanced Macromolecule Transduction Domain (aMTD) peptide for Improvement of Cell-Permeability
gcggcggcgctggtgccggtggtggcgctgctgccg

SEQ ID NO: 249 - DNA, 36 bp, Artificial Sequence
  The polynucleotide sequence that encodes advanced Macromolecule Transduction Domain (aMTD) peptide for Improvement of Cell-Permeability
gcggtggcgctgctgccggcgctgctggcggtgccg

SEQ ID NO: 250 - DNA, 36 bp, Artificial Sequence
  The polynucleotide sequence that encodes advanced Macromolecule Transduction Domain (aMTD) peptide for Improvement of Cell-Permeability
gcggtggtgctggtgccggtgctggcggcggcgccg

SEQ ID NO: 251 - DNA, 36 bp, Artificial Sequence
  The polynucleotide sequence that encodes advanced Macromolecule Transduction Domain (aMTD) peptide for Improvement of Cell-Permeability
gtggtgctggtgctgccggcggcggcggcggtgccg

SEQ ID NO: 252 - DNA, 36 bp, Artificial Sequence
  The polynucleotide sequence that encodes advanced Macromolecule Transduction Domain (aMTD) peptide for Improvement of Cell-Permeability
attgcgctggcggcgccggcgctgattgtggcgccg

SEQ ID NO: 253 - DNA, 36 bp, Artificial Sequence
  The polynucleotide sequence that encodes advanced Macromolecule Transduction Domain (aMTD) peptide for Improvement of Cell-Permeability
attgtggcggtggcgccggcgctggtggcgctgccg

SEQ ID NO: 254 - DNA, 36 bp, Artificial Sequence
  The polynucleotide sequence that encodes advanced Macromolecule Transduction Domain (aMTD) peptide for Improvement of Cell-Permeability
gtggcggcgctgccggtggtggcggtggtggcgccg

SEQ ID NO: 255 - DNA, 36 bp, Artificial Sequence
  The polynucleotide sequence that encodes advanced Macromolecule Transduction Domain (aMTD) peptide for Improvement of Cell-Permeability
ctgctggcggcgccgctggtggtggcggcggtgccg

SEQ ID NO: 256 - DNA, 36 bp, Artificial Sequence
  The polynucleotide sequence that encodes advanced Macromolecule Transduction Domain (aMTD) peptide for Improvement of Cell-Permeability
gcgctggcggtgccggtggcgctgctggtggcgccg

SEQ ID NO: 257 - DNA, 36 bp, Artificial Sequence
  The polynucleotide sequence that encodes advanced Macromolecule Transduction Domain (aMTD) peptide for Improvement of Cell-Permeability
gtggcggcgctgccggtgctgctggcggcgctgccg

SEQ ID NO: 258 - DNA, 36 bp, Artificial Sequence
  The polynucleotide sequence that encodes advanced Macromolecule Transduction Domain (aMTD) peptide for Improvement of Cell-Permeability
gtggcgctgctggcgccggtggcgctggcggtgccg

SEQ ID NO: 259 - DNA, 36 bp, Artificial Sequence
  The polynucleotide sequence that encodes advanced Macromolecule Transduction Domain (aMTD) peptide for Improvement of Cell-Permeability
gcggcgctgctggtgccggcgctggtggcggtgccg

SEQ ID NO: 260 - DNA, 36 bp, Artificial Sequence
  The polynucleotide sequence that encodes advanced Macromolecule Transduction Domain (aMTD) peptide for Improvement of Cell-Permeability
gcgattgtggcgctgccggtggcggtgctggcgccg

SEQ ID NO: 261 - DNA, 36 bp, Artificial Sequence
  The polynucleotide sequence that encodes advanced Macromolecule Transduction Domain (aMTD) peptide for Improvement of Cell-Permeability
attgcgattgtggcgccggtggtggcgctggcgccg

SEQ ID NO: 262 - DNA, 36 bp, Artificial Sequence
  The polynucleotide sequence that encodes advanced Macromolecule Transduction Domain (aMTD) peptide for Improvement of Cell-Permeability
gcggcgctgctgccggcgctggcggcgctgctgccg

SEQ ID NO: 263 - DNA, 36 bp, Artificial Sequence
  The polynucleotide sequence that encodes advanced Macromolecule Transduction Domain (aMTD) peptide for Improvement of Cell-Permeability
gcggtggtgctggcgccggtggcggcggtgctgccg

SEQ ID NO: 264 - DNA, 36 bp, Artificial Sequence
  The polynucleotide sequence that encodes advanced Macromolecule Transduction Domain (aMTD) peptide for Improvement of Cell-Permeability
ctggcggtggcggcgccgctggcgctggcgctgccg

SEQ ID NO: 265 - DNA, 36 bp, Artificial Sequence
  The polynucleotide sequence that encodes advanced Macromolecule Transduction Domain (aMTD) peptide for Improvement of Cell-Permeability
gcggcggtggcggcgccgctgctgctggcgctgccg

SEQ ID NO: 266 - DNA, 36 bp, Artificial Sequence
  The polynucleotide sequence that encodes advanced Macromolecule Transduction Domain (aMTD) peptide for Improvement of Cell-Permeability
ctgctggtgctgccggcggcggcgctggcggcgccg

SEQ ID NO: 267 - DNA, 36 bp, Artificial Sequence
  The polynucleotide sequence that encodes advanced Macromolecule Transduction Domain (aMTD) peptide for Improvement of Cell-Permeability
ctggtggcggtggcgccggtggcggcggtgctgccg

SEQ ID NO: 268 - DNA, 36 bp, Artificial Sequence
  The polynucleotide sequence that encodes advanced Macromolecule Transduction Domain (aMTD) peptide for Improvement of Cell-Permeability
ctggcgctggcgccggcggcgctggcgctgctgccg

SEQ ID NO: 269 - DNA, 36 bp, Artificial Sequence
  The polynucleotide sequence that encodes advanced Macromolecule Transduction Domain (aMTD) peptide for Improvement of Cell-Permeability
gcgctgattgcggcgccgattctggcgctggcgccg

SEQ ID NO: 270 - DNA, 36 bp, Artificial Sequence
  The polynucleotide sequence that encodes advanced Macromolecule Transduction Domain (aMTD) peptide for Improvement of Cell-Permeability
gcggtggtggcggcgccgctggtgctggcgctgccg

SEQ ID NO: 271 - DNA, 36 bp, Artificial Sequence
  The polynucleotide sequence that encodes advanced Macromolecule Transduction Domain (aMTD) peptide for Improvement of Cell-Permeability
ctgctggcgctggcgccggcggcgctgctggcgccg

SEQ ID NO: 272 - DNA, 36 bp, Artificial Sequence
  The polynucleotide sequence that encodes advanced Macromolecule Transduction Domain (aMTD) peptide for Improvement of Cell-Permeability
gcgattgtggcgctgccggcgctggcgctggcgccg

SEQ ID NO: 273 - DNA, 36 bp, Artificial Sequence
  The polynucleotide sequence that encodes advanced Macromolecule Transduction Domain (aMTD) peptide for Improvement of Cell-Permeability
gcggcgattattgtgccggcggcgctgctggcgccg

SEQ ID NO: 274 - DNA, 36 bp, Artificial Sequence
  The polynucleotide sequence that encodes advanced Macromolecule Transduction Domain (aMTD) peptide for Improvement of Cell-Permeability
attgcggtggcgctgccggcgctgattgcggcgccg

SEQ ID NO: 275 - DNA, 36 bp, Artificial Sequence
  The polynucleotide sequence that encodes advanced Macromolecule Transduction Domain (aMTD) peptide for Improvement of Cell-Permeability
gcggtgattgtgctgccggcgctggcggtggcgccg

SEQ ID NO: 276 - DNA, 36 bp, Artificial Sequence
  The polynucleotide sequence that encodes advanced Macromolecule Transduction Domain (aMTD) peptide for Improvement of Cell-Permeability
gcggtgctggcggtgccggcggtgctggtggcgccg

SEQ ID NO: 277 - DNA, 36 bp, Artificial Sequence
  The polynucleotide sequence that encodes advanced Macromolecule Transduction Domain (aMTD) peptide for Improvement of Cell-Permeability
gtgctggcgattgtgccggcggtggcgctggcgccg

SEQ ID NO: 278 - DNA, 36 bp, Artificial Sequence
  The polynucleotide sequence that encodes advanced Macromolecule Transduction Domain (aMTD) peptide for Improvement of Cell-Permeability
ctgctggcggtggtgccggcggtggcgctggcgccg

SEQ ID NO: 279 - DNA, 36 bp, Artificial Sequence
  The polynucleotide sequence that encodes advanced Macromolecule Transduction Domain (aMTD) peptide for Improvement of Cell-Permeability
gcggtgattgcgctgccggcgctgattgcggcgccg

SEQ ID NO: 280 - DNA, 36 bp, Artificial Sequence
  The polynucleotide sequence that encodes advanced Macromolecule Transduction Domain (aMTD) peptide for Improvement of Cell-Permeability
gcggtggtggcgctgccggcggcgctgattgtgccg

SEQ ID NO: 281 - DNA, 36 bp, Artificial Sequence
  The polynucleotide sequence that encodes advanced Macromolecule Transduction Domain (aMTD) peptide for Improvement of Cell-Permeability
ctggcgctggtgctgccggcggcgctggcggcgccg

SEQ ID NO: 282 - DNA, 36 bp, Artificial Sequence
  The polynucleotide sequence that encodes advanced Macromolecule Transduction Domain (aMTD) peptide for Improvement of Cell-Permeability
ctggcggcggtgctgccggcgctgctggcggcgccg

SEQ ID NO: 283 - DNA, 36 bp, Artificial Sequence
  The polynucleotide sequence that encodes advanced Macromolecule Transduction Domain (aMTD) peptide for Improvement of Cell-Permeability
gcgctggcggtgccggtggcgctggcgattgtgccg

SEQ ID NO: 284 - DNA, 36 bp, Artificial Sequence
  The polynucleotide sequence that encodes advanced Macromolecule Transduction Domain (aMTD) peptide for Improvement of Cell-Permeability
gcgctgattgcgccggtggtggcgctggtggcgccg

SEQ ID NO: 285 - DNA, 36 bp, Artificial Sequence
  The polynucleotide sequence that encodes advanced Macromolecule Transduction Domain (aMTD) peptide for Improvement of Cell-Permeability
ctgctggcggcgccggtggtgattgcgctggcgccg

SEQ ID NO: 286 - DNA, 36 bp, Artificial Sequence
  The polynucleotide sequence that encodes advanced Macromolecule Transduction Domain (aMTD) peptide for Improvement of Cell-Permeability
ctggcggcgattgtgccggcgattattgcggtgccg

SEQ ID NO: 287 - DNA, 36 bp, Artificial Sequence
  The polynucleotide sequence that encodes advanced Macromolecule Transduction Domain (aMTD) peptide for Improvement of Cell-Permeability
gcggcgctggtgctgccgctgattattgcggcgccg

SEQ ID NO: 288 - DNA, 36 bp, Artificial Sequence
  The polynucleotide sequence that encodes advanced Macromolecule Transduction Domain (aMTD) peptide for Improvement of Cell-Permeability
ctggcgctggcggtgccggcgctggcggcgctgccg

SEQ ID NO: 289 - DNA, 36 bp, Artificial Sequence
  The polynucleotide sequence that encodes advanced Macromolecule Transduction Domain (aMTD) peptide for Improvement of Cell-Permeability
ctgattgcggcgctgccggcggtggcggcgctgccg

SEQ ID NO: 290 - DNA, 36 bp, Artificial Sequence
  The polynucleotide sequence that encodes advanced Macromolecule Transduction Domain (aMTD) peptide for Improvement of Cell-Permeability
gcgctggcgctggtgccggcgattgcggcgctgccg

SEQ ID NO: 291 - DNA, 36 bp, Artificial Sequence
  The polynucleotide sequence that encodes advanced Macromolecule Transduction Domain (aMTD) peptide for Improvement of Cell-Permeability
gcggcgattctggcgccgattgtggcgctggcgccg

SEQ ID NO: 292 - DNA, 36 bp, Artificial Sequence
  The polynucleotide sequence that encodes advanced Macromolecule Transduction Domain (aMTD) peptide for Improvement of Cell-Permeability
gcgctgctgattgcgccggcggcggtgattgcgccg

SEQ ID NO: 293 - DNA, 36 bp, Artificial Sequence
  The polynucleotide sequence that encodes advanced Macromolecule Transduction Domain (aMTD) peptide for Improvement of Cell-Permeability
gcgattctggcggtgccgattgcggtggtggcgccg

SEQ ID NO: 294 - DNA, 36 bp, Artificial Sequence
  The polynucleotide sequence that encodes advanced Macromolecule Transduction Domain (aMTD) peptide for Improvement of Cell-Permeability
attctggcggcggtgccgattgcgctggcggcgccg

SEQ ID NO: 295 - DNA, 36 bp, Artificial Sequence
  The polynucleotide sequence that encodes advanced Macromolecule Transduction Domain (aMTD) peptide for Improvement of Cell-Permeability
gtggcggcgctgctgccggcggcggcggtgctgccg

SEQ ID NO: 296 - DNA, 36 bp, Artificial Sequence
  The polynucleotide sequence that encodes advanced Macromolecule Transduction Domain (aMTD) peptide for Improvement of Cell-Permeability
gcggcggcggtggtgccggtgctgctggtggcgccg

SEQ ID NO: 297 - DNA, 36 bp, Artificial Sequence
  The polynucleotide sequence that encodes advanced Macromolecule Transduction Domain (aMTD) peptide for Improvement of Cell-Permeability
gcggcgctgctggtgccggcgctggtggcggcgccg

SEQ ID NO: 298 - DNA, 36 bp, Artificial Sequence
  The polynucleotide sequence that encodes advanced Macromolecule Transduction Domain (aMTD) peptide for Improvement of Cell-Permeability
gcggcggtgctgctgccggtggcgctggcggcgccg

SEQ ID NO: 299 - DNA, 36 bp, Artificial Sequence
  The polynucleotide sequence that encodes advanced Macromolecule Transduction Domain (aMTD) peptide for Improvement of Cell-Permeability
gcggcggcgctggcgccggtgctggcgctggtgccg

SEQ ID NO: 300 - DNA, 36 bp, Artificial Sequence
  The polynucleotide sequence that encodes advanced Macromolecule Transduction Domain (aMTD) peptide for Improvement of Cell-Permeability
ctggtgctggtgccgctgctggcggcggcggcgccg

SEQ ID NO: 301 - DNA, 36 bp, Artificial Sequence
  The polynucleotide sequence that encodes advanced Macromolecule Transduction Domain (aMTD) peptide for Improvement of Cell-Permeability
gcgctgattgcggtgccggcgattattgtggcgccg

SEQ ID NO: 302 - DNA, 36 bp, Artificial Sequence
  The polynucleotide sequence that encodes advanced Macromolecule Transduction Domain (aMTD) peptide for Improvement of Cell-Permeability
gcgctggcggtgattccggcggcggcgattctgccg

SEQ ID NO: 303 - DNA, 36 bp, Artificial Sequence
  The polynucleotide sequence that encodes advanced Macromolecule Transduction Domain (aMTD) peptide for Improvement of Cell-Permeability
ctggcggcggcgccggtggtgattgtgattgcgccg

SEQ ID NO: 304 - DNA, 36 bp, Artificial Sequence
  The polynucleotide sequence that encodes advanced Macromolecule Transduction Domain (aMTD) peptide for Improvement of Cell-Permeability
gtgctggcgattgcgccgctgctggcggcggtgccg

SEQ ID NO: 305 - DNA, 36 bp, Artificial Sequence
  The polynucleotide sequence that encodes advanced Macromolecule Transduction Domain (aMTD) peptide for Improvement of Cell-Permeability
gcgctgattgtgctgccggcggcggtggcggtgccg

SEQ ID NO: 306 - DNA, 36 bp, Artificial Sequence
  The polynucleotide sequence that encodes advanced Macromolecule Transduction Domain (aMTD) peptide for Improvement of Cell-Permeability
gtgctggcggtggcgccggcgctgattgtggcgccg

SEQ ID NO: 307 - DNA, 36 bp, Artificial Sequence
  The polynucleotide sequence that encodes advanced Macromolecule Transduction Domain (aMTD) peptide for Improvement of Cell-Permeability
gcggcgctgctggcgccggcgctgattgtggcgccg

SEQ ID NO: 308 - DNA, 36 bp, Artificial Sequence
  The polynucleotide sequence that encodes advanced Macromolecule Transduction Domain (aMTD) peptide for Improvement of Cell-Permeability
gcgctgattgcgccggcggtggcgctgattgtgccg

SEQ ID NO: 309 - DNA, 36 bp, Artificial Sequence
  The polynucleotide sequence that encodes advanced Macromolecule Transduction Domain (aMTD) peptide for Improvement of Cell-Permeability
gcgattgtgctgctgccggcggcggtggtggcgccg

SEQ ID NO: 310 - DNA, 36 bp, Artificial Sequence
  The polynucleotide sequence that encodes advanced Macromolecule Transduction Domain (aMTD) peptide for Improvement of Cell-Permeability
gtgattgcggcgccggtgctggcggtgctggcgccg

SEQ ID NO: 311 - DNA, 36 bp, Artificial Sequence
  The polynucleotide sequence that encodes advanced Macromolecule Transduction Domain (aMTD) peptide for Improvement of Cell-Permeability
ctggcgctggcgccggcgctggcgctgctggcgccg

SEQ ID NO: 312 - DNA, 36 bp, Artificial Sequence
  The polynucleotide sequence that encodes advanced Macromolecule Transduction Domain (aMTD) peptide for Improvement of Cell-Permeability
gcgattattctggcgccgattgcggcgattgcgccg

SEQ ID NO: 313 - DNA, 36 bp, Artificial Sequence
  The polynucleotide sequence that encodes advanced Macromolecule Transduction Domain (aMTD) peptide for Improvement of Cell-Permeability
attgcgctggcggcgccgattctgctggcggcgccg

SEQ ID NO: 314 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcgctggcggtgccg

SEQ ID NO: 315 - DNA, 36 bp, Artificial Sequence
  The polynucleotide sequence that encodes advanced Macromolecule Transduction Domain (aMTD) peptide for Improvement of Cell-Permeability
gtggtggcgattgtgctgccggcgctggcggcgccg

SEQ ID NO: 316 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggtggcgctggcgccg

SEQ ID NO: 317 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcggcgctggtgccg

SEQ ID NO: 318 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcggtggcgctgccg

SEQ ID NO: 319 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcggtgctggcgccg

SEQ ID NO: 320 - DNA, 36 bp, Artificial Sequence
  The polynucleotide sequence that encodes advanced Macromolecule Transduction Domain (aMTD) peptide for Improvement of Cell-Permeability
gtgattgtggcgctggcgccggcggtgctggcgccg

SEQ ID NO: 321 - DNA, 36 bp, Artificial Sequence
  The polynucleotide sequence that encodes advanced Macromolecule Transduction Domain (aMTD) peptide for Improvement of Cell-Permeability
attgtggcggtggcgctgccggcgctggtggcgccg

SEQ ID NO: 322 - DNA, 36 bp, Artificial Sequence
  The polynucleotide sequence that encodes advanced Macromolecule Transduction Domain (aMTD) peptide for Improvement of Cell-Permeability
gcgctgctgattgtggcgccggtggcggtggcgccg

SEQ ID NO: 323 - DNA, 36 bp, Artificial Sequence
  The polynucleotide sequence that encodes advanced Macromolecule Transduction Domain (aMTD) peptide for Improvement of Cell-Permeability
gcggtggtgattgtggcgccggcggtgattgcgccg

SEQ ID NO: 324 - DNA, 36 bp, Artificial Sequence
  The polynucleotide sequence that encodes advanced Macromolecule Transduction Domain (aMTD) peptide for Improvement of Cell-Permeability
gcggtgctggcggtggcgccggcgctgattgtgccg

SEQ ID NO: 325 - DNA, 36 bp, Artificial Sequence
  The polynucleotide sequence that encodes advanced Macromolecule Transduction Domain (aMTD) peptide for Improvement of Cell-Permeability
ctggtggcggcggtggcgccggcgctgattgtgccg

SEQ ID NO: 326 - DNA, 36 bp, Artificial Sequence
  The polynucleotide sequence that encodes advanced Macromolecule Transduction Domain (aMTD) peptide for Improvement of Cell-Permeability
gcggtgattgtggtggcgccggcgctgctggcgccg

SEQ ID NO: 327 - DNA, 36 bp, Artificial Sequence
  The polynucleotide sequence that encodes advanced Macromolecule Transduction Domain (aMTD) peptide for Improvement of Cell-Permeability
gtggtggcgattgtgctgccggcggtggcggcgccg

SEQ ID NO: 328 - DNA, 36 bp, Artificial Sequence
  The polynucleotide sequence that encodes advanced Macromolecule Transduction Domain (aMTD) peptide for Improvement of Cell-Permeability
gcggcggcgctggtgattccggcgattctggcgccg

SEQ ID NO: 329 - DNA, 36 bp, Artificial Sequence
  The polynucleotide sequence that encodes advanced Macromolecule Transduction Domain (aMTD) peptide for Improvement of Cell-Permeability
gtgattgtggcgctggcgccggcgctgctggcgccg

SEQ ID NO: 330 - DNA, 36 bp, Artificial Sequence
  The polynucleotide sequence that encodes advanced Macromolecule Transduction Domain (aMTD) peptide for Improvement of Cell-Permeability
gtgattgtggcgattgcgccggcgctgctggcgccg

SEQ ID NO: 331 - DNA, 36 bp, Artificial Sequence
  The polynucleotide sequence that encodes advanced Macromolecule Transduction Domain (aMTD) peptide for Improvement of Cell-Permeability
attgtggcgattgcggtgccggcgctggtggcgccg

SEQ ID NO: 332 - DNA, 36 bp, Artificial Sequence
  The polynucleotide sequence that encodes advanced Macromolecule Transduction Domain (aMTD) peptide for Improvement of Cell-Permeability
gcggcgctggcggtgattccggcggcgattctgccg

SEQ ID NO: 333 - DNA, 36 bp, Artificial Sequence
  The polynucleotide sequence that encodes advanced Macromolecule Transduction Domain (aMTD) peptide for Improvement of Cell-Permeability
gcgctggcggcggtgattccggcggcgattctgccg

SEQ ID NO: 334 - DNA, 36 bp, Artificial Sequence
  The polynucleotide sequence that encodes advanced Macromolecule Transduction Domain (aMTD) peptide for Improvement of Cell-Permeability
gcggcggcgctggtgattccggcggcgattctgccg

SEQ ID NO: 335 - DNA, 36 bp, Artificial Sequence
  The polynucleotide sequence that encodes advanced Macromolecule Transduction Domain (aMTD) peptide for Improvement of Cell-Permeability
ctggcggcggcggtgattccggcggcgattctgccg

SEQ ID NO: 336 - DNA, 36 bp, Artificial Sequence
  The polynucleotide sequence that encodes advanced Macromolecule Transduction Domain (aMTD) peptide for Improvement of Cell-Permeability
ctggcggcggcggtgattccggtggcgattctgccg

SEQ ID NO: 337 - DNA, 36 bp, Artificial Sequence
  The polynucleotide sequence that encodes advanced Macromolecule Transduction Domain (aMTD) peptide for Improvement of Cell-Permeability
gcggcgattctggcggcgccgctgattgcggtgccg

SEQ ID NO: 338 - DNA, 36 bp, Artificial Sequence
  The polynucleotide sequence that encodes advanced Macromolecule Transduction Domain (aMTD) peptide for Improvement of Cell-Permeability
gtggtggcgattctggcgccgctgctggcggcgccg

SEQ ID NO: 339 - DNA, 36 bp, Artificial Sequence
  The polynucleotide sequence that encodes advanced Macromolecule Transduction Domain (aMTD) peptide for Improvement of Cell-Permeability
gcggtggtggtggcggcgccggtgctggcgctgccg

SEQ ID NO: 340 - DNA, 36 bp, Artificial Sequence
  The polynucleotide sequence that encodes advanced Macromolecule Transduction Domain (aMTD) peptide for Improvement of Cell-Permeability
gcggtggtggcgattgcgccggtgctggcgctgccg

SEQ ID NO: 341 - DNA, 36 bp, Artificial Sequence
  The polynucleotide sequence that encodes advanced Macromolecule Transduction Domain (aMTD) peptide for Improvement of Cell-Permeability
gcgctggcggcgctggtgccggcggtgctggtgccg

SEQ ID NO: 342 - DNA, 36 bp, Artificial Sequence
  The polynucleotide sequence that encodes advanced Macromolecule Transduction Domain (aMTD) peptide for Improvement of Cell-Permeability
gcgctggcggcgctggtgccggtggcgctggtgccg

SEQ ID NO: 343 - DNA, 36 bp, Artificial Sequence
  The polynucleotide sequence that encodes advanced Macromolecule Transduction Domain (aMTD) peptide for Improvement of Cell-Permeability
ctggcggcggcgctggtgccggtggcgctggtgccg

SEQ ID NO: 344 - DNA, 36 bp, Artificial Sequence
  The polynucleotide sequence that encodes advanced Macromolecule Transduction Domain (aMTD) peptide for Improvement of Cell-Permeability
gcgctggcggcgctggtgccggcgctggtggtgccg

SEQ ID NO: 345 - DNA, 36 bp, Artificial Sequence
  The polynucleotide sequence that encodes advanced Macromolecule Transduction Domain (aMTD) peptide for Improvement of Cell-Permeability
attgcggcggtgattgtgccggcggtggcgctgccg

SEQ ID NO: 346 - DNA, 36 bp, Artificial Sequence
  The polynucleotide sequence that encodes advanced Macromolecule Transduction Domain (aMTD) peptide for Improvement of Cell-Permeability
attgcggcggtgctggtgccggcggtggcgctgccg

SEQ ID NO: 347 - DNA, 36 bp, Artificial Sequence
  The polynucleotide sequence that encodes advanced Macromolecule Transduction Domain (aMTD) peptide for Improvement of Cell-Permeability
gcggtggcgattctggtgccgctgctggcggcgccg

SEQ ID NO: 348 - DNA, 36 bp, Artificial Sequence
  The polynucleotide sequence that encodes advanced Macromolecule Transduction Domain (aMTD) peptide for Improvement of Cell-Permeability
gcggtggtgattctggtgccgctggcggcggcgccg

SEQ ID NO: 349 - DNA, 36 bp, Artificial Sequence
  The polynucleotide sequence that encodes advanced Macromolecule Transduction Domain (aMTD) peptide for Improvement of Cell-Permeability
attgcggcggtgattgtgccggtggcggcgctgccg

SEQ ID NO: 350 - DNA, 36 bp, Artificial Sequence
  The polynucleotide sequence that encodes advanced Macromolecule Transduction Domain (aMTD) peptide for Improvement of Cell-Permeability
gcgattgcgattgcgattgtgccggtggcgctgccg

SEQ ID NO: 351 - DNA, 36 bp, Artificial Sequence
  The polynucleotide sequence that encodes advanced Macromolecule Transduction Domain (aMTD) peptide for Improvement of Cell-Permeability
attctggcggtggcggcgattccggtggcggtgccg

SEQ ID NO: 352 - DNA, 36 bp, Artificial Sequence
  The polynucleotide sequence that encodes advanced Macromolecule Transduction Domain (aMTD) peptide for Improvement of Cell-Permeability
attctggcggcggcgattattccggcggcgctgccg

SEQ ID NO: 353 - DNA, 36 bp, Artificial Sequence
  The polynucleotide sequence that encodes advanced Macromolecule Transduction Domain (aMTD) peptide for Improvement of Cell-Permeability
ctggcggtggtgctggcggcgccggcgattgtgccg

SEQ ID NO: 354 - DNA, 36 bp, Artificial Sequence
  The polynucleotide sequence that encodes advanced Macromolecule Transduction Domain (aMTD) peptide for Improvement of Cell-Permeability
gcgattctggcggcgattgtgccgctggcggtgccg

SEQ ID NO: 355 - DNA, 36 bp, Artificial Sequence
  The polynucleotide sequence that encodes advanced Macromolecule Transduction Domain (aMTD) peptide for Improvement of Cell-Permeability
gtgattgtggcgctggcggtgccggcgctggcgccg

SEQ ID NO: 356 - DNA, 36 bp, Artificial Sequence
  The polynucleotide sequence that encodes advanced Macromolecule Transduction Domain (aMTD) peptide for Improvement of Cell-Permeability
gcgattgtggcgctggcggtgccggtgctggcgccg

SEQ ID NO: 357 - DNA, 36 bp, Artificial Sequence
  The polynucleotide sequence that encodes advanced Macromolecule Transduction Domain (aMTD) peptide for Improvement of Cell-Permeability
gcggcgattattattgtgctgccggcggcgctgccg

SEQ ID NO: 358 - DNA, 36 bp, Artificial Sequence
  The polynucleotide sequence that encodes advanced Macromolecule Transduction Domain (aMTD) peptide for Improvement of Cell-Permeability
ctgattgtggcgctggcggtgccggcgctggcgccg

SEQ ID NO: 359 - DNA, 36 bp, Artificial Sequence
  The polynucleotide sequence that encodes advanced Macromolecule Transduction Domain (aMTD) peptide for Improvement of Cell-Permeability
gcgattattattgtgattgcgccggcggcggcgccg

SEQ ID NO: 360 - DNA, 36 bp, Artificial Sequence
  The polynucleotide sequence that encodes advanced Macromolecule Transduction Domain (aMTD) peptide for Improvement of Cell-Permeability
ctggcggcgctgattgtggtgccggcggtggcgccg

SEQ ID NO: 361 - DNA, 36 bp, Artificial Sequence
  The polynucleotide sequence that encodes advanced Macromolecule Transduction Domain (aMTD) peptide for Improvement of Cell-Permeability
gcgctgctggtgattgcggtgccggcggtggcgccg

SEQ ID NO: 362 - DNA, 36 bp, Artificial Sequence
  The polynucleotide sequence that encodes advanced Macromolecule Transduction Domain (aMTD) peptide for Improvement of Cell-Permeability
gcggtggcgctgattgtggtgccggcgctggcgccg

SEQ ID NO: 363 - DNA, 36 bp, Artificial Sequence
  The polynucleotide sequence that encodes advanced Macromolecule Transduction Domain (aMTD) peptide for Improvement of Cell-Permeability
gcgctggcgattgtggtggcgccggtggcggtgccg

SEQ ID NO: 364 - DNA, 36 bp, Artificial Sequence
  The polynucleotide sequence that encodes advanced Macromolecule Transduction Domain (aMTD) peptide for Improvement of Cell-Permeability
ctgctggcgctgattattgcgccggcggcggcgccg

SEQ ID NO: 365 - DNA, 36 bp, Artificial Sequence
  The polynucleotide sequence that encodes advanced Macromolecule Transduction Domain (aMTD) peptide for Improvement of Cell-Permeability
gcgctggcgctgattattgtgccggcggtggcgccg

SEQ ID NO: 366 - DNA, 36 bp, Artificial Sequence
  The polynucleotide sequence that encodes advanced Macromolecule Transduction Domain (aMTD) peptide for Improvement of Cell-Permeability
ctgctggcggcgctgattgcgccggcggcgctgccg

SEQ ID NO: 367 - DNA, 36 bp, Artificial Sequence
  The polynucleotide sequence that encodes advanced Macromolecule Transduction Domain (aMTD) peptide for Improvement of Cell-Permeability
attgtggcgctgattgtggcgccggcggcggtgccg

SEQ ID NO: 368 - DNA, 36 bp, Artificial Sequence
  The polynucleotide sequence that encodes advanced Macromolecule Transduction Domain (aMTD) peptide for Improvement of Cell-Permeability
gtggtgctggtgctggcggcgccggcggcggtgccg

SEQ ID NO: 369 - DNA, 36 bp, Artificial Sequence
  The polynucleotide sequence that encodes advanced Macromolecule Transduction Domain (aMTD) peptide for Improvement of Cell-Permeability
gcggcggtggcgattgtgctgccggcggtggtgccg

SEQ ID NO: 370 - DNA, 36 bp, Artificial Sequence
  The polynucleotide sequence that encodes advanced Macromolecule Transduction Domain (aMTD) peptide for Improvement of Cell-Permeability
gcgctgattgcggcgattgtgccggcgctggtgccg

SEQ ID NO: 371 - DNA, 36 bp, Artificial Sequence
  The polynucleotide sequence that encodes advanced Macromolecule Transduction Domain (aMTD) peptide for Improvement of Cell-Permeability
gcgctggcggtgattgtggtgccggcgctggcgccg

SEQ ID NO: 372 - DNA, 36 bp, Artificial Sequence
  The polynucleotide sequence that encodes advanced Macromolecule Transduction Domain (aMTD) peptide for Improvement of Cell-Permeability
gtggcgattgcgctgattgtgccggcgctggcgccg

SEQ ID NO: 373 - DNA, 36 bp, Artificial Sequence
  The polynucleotide sequence that encodes advanced Macromolecule Transduction Domain (aMTD) peptide for Improvement of Cell-Permeability
gtggcgattgtgctggtggcgccggcggtggcgccg

SEQ ID NO: 374 - DNA, 36 bp, Artificial Sequence
  The polynucleotide sequence that encodes advanced Macromolecule Transduction Domain (aMTD) peptide for Improvement of Cell-Permeability
gtggcggtggcgctgattgtgccggcgctggcgccg

SEQ ID NO: 375 - DNA, 36 bp, Artificial Sequence
  The polynucleotide sequence that encodes advanced Macromolecule Transduction Domain (aMTD) peptide for Improvement of Cell-Permeability
gcggtgattctggcgctggcgccgattgtggcgccg

SEQ ID NO: 376 - DNA, 36 bp, Artificial Sequence
  The polynucleotide sequence that encodes advanced Macromolecule Transduction Domain (aMTD) peptide for Improvement of Cell-Permeability
gcgctgattgtggcgattgcgccggcgctggtgccg

SEQ ID NO: 377 - DNA, 36 bp, Artificial Sequence
  The polynucleotide sequence that encodes advanced Macromolecule Transduction Domain (aMTD) peptide for Improvement of Cell-Permeability
gcggcgattctgattgcggtgccgattgcggcgccg

SEQ ID NO: 378 - DNA, 36 bp, Artificial Sequence
  The polynucleotide sequence that encodes advanced Macromolecule Transduction Domain (aMTD) peptide for Improvement of Cell-Permeability
gtgattgtggcgctggcggcgccggtgctggcgccg

SEQ ID NO: 379 - DNA, 36 bp, Artificial Sequence
  The polynucleotide sequence that encodes advanced Macromolecule Transduction Domain (aMTD) peptide for Improvement of Cell-Permeability
gtgctggtggcgctggcggcgccggtgattgcgccg

SEQ ID NO: 380 - DNA, 36 bp, Artificial Sequence
  The polynucleotide sequence that encodes advanced Macromolecule Transduction Domain (aMTD) peptide for Improvement of Cell-Permeability
gtggcgctgattgcggtggcgccggcggtggtgccg

SEQ ID NO: 381 - DNA, 36 bp, Artificial Sequence
  The polynucleotide sequence that encodes advanced Macromolecule Transduction Domain (aMTD) peptide for Improvement of Cell-Permeability
gtgattgcggcggtgctggcgccggtggcggtgccg

SEQ ID NO: 382 - DNA, 36 bp, Artificial Sequence
  The polynucleotide sequence that encodes advanced Macromolecule Transduction Domain (aMTD) peptide for Improvement of Cell-Permeability
gcgctgattgtgctggcggcgccggtggcggtgccg

SEQ ID NO: 383 - DNA, 36 bp, Artificial Sequence
  The polynucleotide sequence that encodes advanced Macromolecule Transduction Domain (aMTD) peptide for Improvement of Cell-Permeability
gtggcggcggcgattgcgctgccggcgattgtgccg

SEQ ID NO: 384 - DNA, 36 bp, Artificial Sequence
  The polynucleotide sequence that encodes advanced Macromolecule Transduction Domain (aMTD) peptide for Improvement of Cell-Permeability
attctggcggcggcggcggcgccgctgattgtgccg

SEQ ID NO: 385 - DNA, 36 bp, Artificial Sequence
  The polynucleotide sequence that encodes advanced Macromolecule Transduction Domain (aMTD) peptide for Improvement of Cell-Permeability
ctggcgctggtgctggcggcgccggcgattgtgccg

SEQ ID NO: 386 - DNA, 36 bp, Artificial Sequence
  The polynucleotide sequence that encodes advanced Macromolecule Transduction Domain (aMTD) peptide for Improvement of Cell-Permeability
gcgctggcggtggtggcgctgccggcgattgtgccg

SEQ ID NO: 387 - DNA, 36 bp, Artificial Sequence
  The polynucleotide sequence that encodes advanced Macromolecule Transduction Domain (aMTD) peptide for Improvement of Cell-Permeability
gcggcgattctggcgccgattgtggcggcgctgccg

SEQ ID NO: 388 - DNA, 36 bp, Artificial Sequence
  The polynucleotide sequence that encodes advanced Macromolecule Transduction Domain (aMTD) peptide for Improvement of Cell-Permeability
attctgattgcgattgcgattccggcggcggcgccg

SEQ ID NO: 389 - DNA, 36 bp, Artificial Sequence
  The polynucleotide sequence that encodes advanced Macromolecule Transduction Domain (aMTD) peptide for Improvement of Cell-Permeability
ctggcgattgtgctggcggcgccggtggcggtgccg

SEQ ID NO: 390 - DNA, 36 bp, Artificial Sequence
  The polynucleotide sequence that encodes advanced Macromolecule Transduction Domain (aMTD) peptide for Improvement of Cell-Permeability
gcggcgattgcgattattgcgccggcgattgtgccg

SEQ ID NO: 391 - DNA, 36 bp, Artificial Sequence
  The polynucleotide sequence that encodes advanced Macromolecule Transduction Domain (aMTD) peptide for Improvement of Cell-Permeability
ctggcggtggcgattgtggcgccggcgctggtgccg

SEQ ID NO: 392 - DNA, 36 bp, Artificial Sequence
  The polynucleotide sequence that encodes advanced Macromolecule Transduction Domain (aMTD) peptide for Improvement of Cell-Permeability
ctggcgattgtgctggcggcgccggcggtgctgccg

SEQ ID NO: 393 - DNA, 36 bp, Artificial Sequence
  The polynucleotide sequence that encodes advanced Macromolecule Transduction Domain (aMTD) peptide for Improvement of Cell-Permeability
gcggcgattgtgctggcgctgccggcggtgctgccg

SEQ ID NO: 394 - DNA, 36 bp, Artificial Sequence
  The polynucleotide sequence that encodes advanced Macromolecule Transduction Domain (aMTD) peptide for Improvement of Cell-Permeability
gcgctgctggtggcggtgctgccggcggcgctgccg

SEQ ID NO: 395 - DNA, 36 bp, Artificial Sequence
  The polynucleotide sequence that encodes advanced Macromolecule Transduction Domain (aMTD) peptide for Improvement of Cell-Permeability
gcggcgctggtggcggtgctgccggtggcgctgccg

SEQ ID NO: 396 - DNA, 36 bp, Artificial Sequence
  The polynucleotide sequence that encodes advanced Macromolecule Transduction Domain (aMTD) peptide for Improvement of Cell-Permeability
gcgattctggcggtggcgctgccgctgctggcgccg

SEQ ID NO: 397 - DNA, 36 bp, Artificial Sequence
  The polynucleotide sequence that encodes advanced Macromolecule Transduction Domain (aMTD) peptide for Improvement of Cell-Permeability
attgtggcggtggcgctggtgccggcgctggcgccg

SEQ ID NO: 398 - DNA, 36 bp, Artificial Sequence
  The polynucleotide sequence that encodes advanced Macromolecule Transduction Domain (aMTD) peptide for Improvement of Cell-Permeability
attgtggcggtggcgctgctgccggcgctggcgccg

SEQ ID NO: 399 - DNA, 36 bp, Artificial Sequence
  The polynucleotide sequence that encodes advanced Macromolecule Transduction Domain (aMTD) peptide for Improvement of Cell-Permeability
attgtggcggtggcgctgctgccggcggtggcgccg

SEQ ID NO: 400 - DNA, 36 bp, Artificial Sequence
  The polynucleotide sequence that encodes advanced Macromolecule Transduction Domain (aMTD) peptide for Improvement of Cell-Permeability
attgtggcgctggcggtgctgccggcggtggcgccg

SEQ ID NO: 401 - DNA, 36 bp, Artificial Sequence
  The polynucleotide sequence that encodes advanced Macromolecule Transduction Domain (aMTD) peptide for Improvement of Cell-Permeability
gtggcggtgctggcggtgctgccggcgctggcgccg

SEQ ID NO: 402 - DNA, 36 bp, Artificial Sequence
  The polynucleotide sequence that encodes advanced Macromolecule Transduction Domain (aMTD) peptide for Improvement of Cell-Permeability
attgcggtgctggcggtggcgccggcggtgctgccg

SEQ ID NO: 403 - DNA, 36 bp, Artificial Sequence
  The polynucleotide sequence that encodes advanced Macromolecule Transduction Domain (aMTD) peptide for Improvement of Cell-Permeability
ctggcggtggcgattattgcgccggcggtggcgccg

SEQ ID NO: 404 - DNA, 36 bp, Artificial Sequence
  The polynucleotide sequence that encodes advanced Macromolecule Transduction Domain (aMTD) peptide for Improvement of Cell-Permeability
gtggcgctggcgattgcgctgccggcggtgctgccg

SEQ ID NO: 405 - DNA, 36 bp, Artificial Sequence
  The polynucleotide sequence that encodes advanced Macromolecule Transduction Domain (aMTD) peptide for Improvement of Cell-Permeability
gcgattgcgattgcgctggtgccggtggcgctgccg

SEQ ID NO: 406 - DNA, 36 bp, Artificial Sequence
  The polynucleotide sequence that encodes advanced Macromolecule Transduction Domain (aMTD) peptide for Improvement of Cell-Permeability
gcggcggtggtgattgtggcgccggtggcgctgccg

SEQ ID NO: 407 - DNA, 36 bp, Artificial Sequence
  The polynucleotide sequence that encodes advanced Macromolecule Transduction Domain (aMTD) peptide for Improvement of Cell-Permeability
gcggcgattctggcgattgtggcgccgctggcgccg

SEQ ID NO: 408 - DNA, 36 bp, Artificial Sequence
  The polynucleotide sequence that encodes advanced Macromolecule Transduction Domain (aMTD) peptide for Improvement of Cell-Permeability
gtggcgctgctggcgattgcgccggcgctggcgccg

SEQ ID NO: 409 - DNA, 36 bp, Artificial Sequence
  The polynucleotide sequence that encodes advanced Macromolecule Transduction Domain (aMTD) peptide for Improvement of Cell-Permeability
gtggcggtgctgattgcggtgccggcgctggcgccg

SEQ ID NO: 410 - DNA, 36 bp, Artificial Sequence
  The polynucleotide sequence that encodes advanced Macromolecule Transduction Domain (aMTD) peptide for Improvement of Cell-Permeability
gcggtggcgctggcggtgctgccggcggtggtgccg

SEQ ID NO: 411 - DNA, 36 bp, Artificial Sequence
  The polynucleotide sequence that encodes advanced Macromolecule Transduction Domain (aMTD) peptide for Improvement of Cell-Permeability
gcggtggcgctggcggtggtgccggcggtgctgccg

SEQ ID NO: 412 - DNA, 36 bp, Artificial Sequence
  The polynucleotide sequence that encodes advanced Macromolecule Transduction Domain (aMTD) peptide for Improvement of Cell-Permeability
attgtggtgattgcggtggcgccggcggtggcgccg

SEQ ID NO: 413 - DNA, 36 bp, Artificial Sequence
  The polynucleotide sequence that encodes advanced Macromolecule Transduction Domain (aMTD) peptide for Improvement of Cell-Permeability
attgtggtggcggcggtggtgccggcgctggcgccg

SEQ ID NO: 414 - DNA, 36 bp, Artificial Sequence
  The polynucleotide sequence that encodes advanced Macromolecule Transduction Domain (aMTD) peptide for Improvement of Cell-Permeability
attgtggcgctggtgccggcggtggcgattgcgccg

SEQ ID NO: 415 - DNA, 36 bp, Artificial Sequence
  The polynucleotide sequence that encodes advanced Macromolecule Transduction Domain (aMTD) peptide for Improvement of Cell-Permeability
gtggcggcgctgccggcggtggcgctggtggtgccg

SEQ ID NO: 416 - DNA, 36 bp, Artificial Sequence
  The polynucleotide sequence that encodes advanced Macromolecule Transduction Domain (aMTD) peptide for Improvement of Cell-Permeability
ctggtggcgattgcgccgctggcggtgctggcgccg

SEQ ID NO: 417 - DNA, 36 bp, Artificial Sequence
  The polynucleotide sequence that encodes advanced Macromolecule Transduction Domain (aMTD) peptide for Improvement of Cell-Permeability
gcggtggcgctggtgccggtgattgtggcggcgccg

SEQ ID NO: 418 - DNA, 36 bp, Artificial Sequence
  The polynucleotide sequence that encodes advanced Macromolecule Transduction Domain (aMTD) peptide for Improvement of Cell-Permeability
gcgattgcggtggcgattgcgccggtggcgctgccg

SEQ ID NO: 419 - DNA, 36 bp, Artificial Sequence
  The polynucleotide sequence that encodes advanced Macromolecule Transduction Domain (aMTD) peptide for Improvement of Cell-Permeability
gcgattgcgctggcggtgccggtgctggcgctgccg

SEQ ID NO: 420 - DNA, 36 bp, Artificial Sequence
  The polynucleotide sequence that encodes advanced Macromolecule Transduction Domain (aMTD) peptide for Improvement of Cell-Permeability
ctggtgctgattgcggcggcgccgattgcgctgccg

SEQ ID NO: 421 - DNA, 36 bp, Artificial Sequence
  The polynucleotide sequence that encodes advanced Macromolecule Transduction Domain (aMTD) peptide for Improvement of Cell-Permeability
ctggtggcgctggcggtgccggcggcggtgctgccg

SEQ ID NO: 422 - DNA, 36 bp, Artificial Sequence
  The polynucleotide sequence that encodes advanced Macromolecule Transduction Domain (aMTD) peptide for Improvement of Cell-Permeability
gcggtggcgctggcggtgccggcgctggtgctgccg

SEQ ID NO: 423 - DNA, 36 bp, Artificial Sequence
  The polynucleotide sequence that encodes advanced Macromolecule Transduction Domain (aMTD) peptide for Improvement of Cell-Permeability
ctggtggtgctggcggcggcgccgctggcggtgccg

SEQ ID NO: 424 - DNA, 36 bp, Artificial Sequence
  The polynucleotide sequence that encodes advanced Macromolecule Transduction Domain (aMTD) peptide for Improvement of Cell-Permeability
ctgattgtgctggcggcgccggcgctggcggcgccg

SEQ ID NO: 425 - DNA, 36 bp, Artificial Sequence
  The polynucleotide sequence that encodes advanced Macromolecule Transduction Domain (aMTD) peptide for Improvement of Cell-Permeability
gtgattgtgctggcggcgccggcgctggcggcgccg

SEQ ID NO: 426 - DNA, 36 bp, Artificial Sequence
  The polynucleotide sequence that encodes advanced Macromolecule Transduction Domain (aMTD) peptide for Improvement of Cell-Permeability
gcggtggtgctggcggtgccggcgctggcggtgccg

SEQ ID NO: 427 - DNA, 36 bp, Artificial Sequence
  The polynucleotide sequence that encodes advanced Macromolecule Transduction Domain (aMTD) peptide for Improvement of Cell-Permeability
ctgattattgtggcggcggcgccggcggtggcgccg

SEQ ID NO: 428 - DNA, 36 bp, Artificial Sequence
  The polynucleotide sequence that encodes advanced Macromolecule Transduction Domain (aMTD) peptide for Improvement of Cell-Permeability
attgtggcggtgattgtggcgccggcggtggcgccg

SEQ ID NO: 429 - DNA, 36 bp, Artificial Sequence
  The polynucleotide sequence that encodes advanced Macromolecule Transduction Domain (aMTD) peptide for Improvement of Cell-Permeability
ctggtggcgctggcggcgccgattattgcggtgccg

SEQ ID NO: 430 - DNA, 36 bp, Artificial Sequence
  The polynucleotide sequence that encodes advanced Macromolecule Transduction Domain (aMTD) peptide for Improvement of Cell-Permeability
attgcggcggtgctggcggcgccggcgctggtgccg

SEQ ID NO: 431 - DNA, 36 bp, Artificial Sequence
  The polynucleotide sequence that encodes advanced Macromolecule Transduction Domain (aMTD) peptide for Improvement of Cell-Permeability
attgcgctgctggcggcgccgattattgcggtgccg

SEQ ID NO: 432 - DNA, 36 bp, Artificial Sequence
  The polynucleotide sequence that encodes advanced Macromolecule Transduction Domain (aMTD) peptide for Improvement of Cell-Permeability
gcggcgctggcgctggtggcgccggtgattgtgccg

SEQ ID NO: 433 - DNA, 36 bp, Artificial Sequence
  The polynucleotide sequence that encodes advanced Macromolecule Transduction Domain (aMTD) peptide for Improvement of Cell-Permeability
attgcgctggtggcggcgccggtggcgctggtgccg

SEQ ID NO: 434 - DNA, 36 bp, Artificial Sequence
  The polynucleotide sequence that encodes advanced Macromolecule Transduction Domain (aMTD) peptide for Improvement of Cell-Permeability
attattgtggcggtggcgccggcggcgattgtgccg

SEQ ID NO: 435 - DNA, 36 bp, Artificial Sequence
  The polynucleotide sequence that encodes advanced Macromolecule Transduction Domain (aMTD) peptide for Improvement of Cell-Permeability
gcggtggcggcgattgtgccggtgattgtggcgccg

SEQ ID NO: 436 - DNA, 36 bp, Artificial Sequence
  The polynucleotide sequence that encodes advanced Macromolecule Transduction Domain (aMTD) peptide for Improvement of Cell-Permeability
gcggtgctggtgctggtggcgccggcggcggcgccg

SEQ ID NO: 437 - DNA, 36 bp, Artificial Sequence
  The polynucleotide sequence that encodes advanced Macromolecule Transduction Domain (aMTD) peptide for Improvement of Cell-Permeability
gtggtggcgctgctggcgccgctgattgcggcgccg

SEQ ID NO: 438 - DNA, 36 bp, Artificial Sequence
  The polynucleotide sequence that encodes advanced Macromolecule Transduction Domain (aMTD) peptide for Improvement of Cell-Permeability
gcggcggtggtgattgcgccgctgctggcggtgccg

SEQ ID NO: 439 - DNA, 36 bp, Artificial Sequence
  The polynucleotide sequence that encodes advanced Macromolecule Transduction Domain (aMTD) peptide for Improvement of Cell-Permeability
attgcggtggcggtggcggcgccgctgctggtgccg

SEQ ID NO: 440 - DNA, 36 bp, Artificial Sequence
  The polynucleotide sequence that encodes advanced Macromolecule Transduction Domain (aMTD) peptide for Improvement of Cell-Permeability
ctggtggcgattgtggtgctgccggcggtggcgccg

SEQ ID NO: 441 - DNA, 36 bp, Artificial Sequence
  The polynucleotide sequence that encodes advanced Macromolecule Transduction Domain (aMTD) peptide for Improvement of Cell-Permeability
gcggtggcgattgtggtgctgccggcggtggcgccg

SEQ ID NO: 442 - DNA, 36 bp, Artificial Sequence
  The polynucleotide sequence that encodes advanced Macromolecule Transduction Domain (aMTD) peptide for Improvement of Cell-Permeability
gcggtgattctgctggcgccgctgattgcggcgccg

SEQ ID NO: 443 - DNA, 36 bp, Artificial Sequence
  The polynucleotide sequence that encodes advanced Macromolecule Transduction Domain (aMTD) peptide for Improvement of Cell-Permeability
ctggtgattgcgctggcggcgccggtggcgctgccg

SEQ ID NO: 444 - DNA, 36 bp, Artificial Sequence
  The polynucleotide sequence that encodes advanced Macromolecule Transduction Domain (aMTD) peptide for Improvement of Cell-Permeability
gtgctggcggtggtgctgccggcggtggcgctgccg

SEQ ID NO: 445 - DNA, 36 bp, Artificial Sequence
  The polynucleotide sequence that encodes advanced Macromolecule Transduction Domain (aMTD) peptide for Improvement of Cell-Permeability
gtgctggcggtggcggcgccggcggtgctgctgccg

SEQ ID NO: 446 - DNA, 36 bp, Artificial Sequence
  The polynucleotide sequence that encodes advanced Macromolecule Transduction Domain (aMTD) peptide for Improvement of Cell-Permeability
gcggcggtggtgctgctgccgattattgcggcgccg

SEQ ID NO: 447 - DNA, 36 bp, Artificial Sequence
  The polynucleotide sequence that encodes advanced Macromolecule Transduction Domain (aMTD) peptide for Improvement of Cell-Permeability
gcgctgctggtgattgcgccggcgattgcggtgccg

SEQ ID NO: 448 - DNA, 36 bp, Artificial Sequence
  The polynucleotide sequence that encodes advanced Macromolecule Transduction Domain (aMTD) peptide for Improvement of Cell-Permeability
gcggtgctggtgattgcggtgccggcgattgcgccg

SEQ ID NO: 449 - DNA, 36 bp, Artificial Sequence
  The polynucleotide sequence that encodes advanced Macromolecule Transduction Domain (aMTD) peptide for Improvement of Cell-Permeability
gcgctgctggtggtgattgcgccgctggcggcgccg

SEQ ID NO: 450 - DNA, 36 bp, Artificial Sequence
  The polynucleotide sequence that encodes advanced Macromolecule Transduction Domain (aMTD) peptide for Improvement of Cell-Permeability
gtgctggtggcggcgattctgccggcggcgattccg

SEQ ID NO: 451 - DNA, 36 bp, Artificial Sequence
  The polynucleotide sequence that encodes advanced Macromolecule Transduction Domain (aMTD) peptide for Improvement of Cell-Permeability
gtgctggtggcggcggtgctgccgattgcggcgccg

SEQ ID NO: 452 - DNA, 36 bp, Artificial Sequence
  The polynucleotide sequence that encodes advanced Macromolecule Transduction Domain (aMTD) peptide for Improvement of Cell-Permeability
gtgctggcggcggcggtgctgccgctggtggtgccg

SEQ ID NO: 453 - DNA, 36 bp, Artificial Sequence
  The polynucleotide sequence that encodes advanced Macromolecule Transduction Domain (aMTD) peptide for Improvement of Cell-Permeability
gcgattgcgattgtggtgccggcggtggcggtgccg

SEQ ID NO: 454 - DNA, 36 bp, Artificial Sequence
  The polynucleotide sequence that encodes advanced Macromolecule Transduction Domain (aMTD) peptide for Improvement of Cell-Permeability
gtggcgattattgcggtgccggcggtggtggcgccg

SEQ ID NO: 455 - DNA, 36 bp, Artificial Sequence
  The polynucleotide sequence that encodes advanced Macromolecule Transduction Domain (aMTD) peptide for Improvement of Cell-Permeability
attgtggcgctggtggcgccggcggcggtggtgccg

SEQ ID NO: 456 - DNA, 36 bp, Artificial Sequence
  The polynucleotide sequence that encodes advanced Macromolecule Transduction Domain (aMTD) peptide for Improvement of Cell-Permeability
gcggcgattgtgctgctgccggcggtggtggtgccg

SEQ ID NO: 457 - DNA, 36 bp, Artificial Sequence
  The polynucleotide sequence that encodes advanced Macromolecule Transduction Domain (aMTD) peptide for Improvement of Cell-Permeability
gcggcgctgattgtggtgccggcggtggcggtgccg

SEQ ID NO: 458 - DNA, 36 bp, Artificial Sequence
  The polynucleotide sequence that encodes advanced Macromolecule Transduction Domain (aMTD) peptide for Improvement of Cell-Permeability
gcgattgcgctggtggtgccggcggtggcggtgccg

SEQ ID NO: 459 - DNA, 36 bp, Artificial Sequence
  The polynucleotide sequence that encodes advanced Macromolecule Transduction Domain (aMTD) peptide for Improvement of Cell-Permeability
ctggcgattgtgccggcggcgattgcggcgctgccg

SEQ ID NO: 460 - DNA, 36 bp, Artificial Sequence
  The polynucleotide sequence that encodes advanced Macromolecule Transduction Domain (aMTD) peptide for Improvement of Cell-Permeability
ctggtggcgattgcgccggcggtggcggtgctgccg

SEQ ID NO: 461 - DNA, 36 bp, Artificial Sequence
  The polynucleotide sequence that encodes advanced Macromolecule Transduction Domain (aMTD) peptide for Improvement of Cell-Permeability
gtgctggcggtggcgccggcggtggcggtgctgccg

SEQ ID NO: 462 - DNA, 36 bp, Artificial Sequence
  The polynucleotide sequence that encodes advanced Macromolecule Transduction Domain (aMTD) peptide for Improvement of Cell-Permeability
attctggcggtggtggcgattccggcggcggcgccg

SEQ ID NO: 463 - DNA, 36 bp, Artificial Sequence
  The polynucleotide sequence that encodes advanced Macromolecule Transduction Domain (aMTD) peptide for Improvement of Cell-Permeability
attctggtggcggcggcgccgattgcggcgctgccg

SEQ ID NO: 464 - DNA, 36 bp, Artificial Sequence
  The polynucleotide sequence that encodes advanced Macromolecule Transduction Domain (aMTD) peptide for Improvement of Cell-Permeability
attctggcggtggcggcgattccggcggcgctgccg

SEQ ID NO: 465 - DNA, 36 bp, Artificial Sequence
  The polynucleotide sequence that encodes advanced Macromolecule Transduction Domain (aMTD) peptide for Improvement of Cell-Permeability
gtgattgcgattccggcgattctggcggcggcgccg

SEQ ID NO: 466 - DNA, 36 bp, Artificial Sequence
  The polynucleotide sequence that encodes advanced Macromolecule Transduction Domain (aMTD) peptide for Improvement of Cell-Permeability
gcgattattattgtggtgccggcgattgcggcgccg

SEQ ID NO: 467 - DNA, 36 bp, Artificial Sequence
  The polynucleotide sequence that encodes advanced Macromolecule Transduction Domain (aMTD) peptide for Improvement of Cell-Permeability
gcgattctgattgtggtggcgccgattgcggcgccg

SEQ ID NO: 468 - DNA, 36 bp, Artificial Sequence
  The polynucleotide sequence that encodes advanced Macromolecule Transduction Domain (aMTD) peptide for Improvement of Cell-Permeability
gcggtgattgtgccggtggcgattattgcggcgccg

SEQ ID NO: 469 - DNA, 36 bp, Artificial Sequence
  The polynucleotide sequence that encodes advanced Macromolecule Transduction Domain (aMTD) peptide for Improvement of Cell-Permeability
gcggtggtgattgcgctgccggcggtggtggcgccg

SEQ ID NO: 470 - DNA, 36 bp, Artificial Sequence
  The polynucleotide sequence that encodes advanced Macromolecule Transduction Domain (aMTD) peptide for Improvement of Cell-Permeability
gcgctggtggcggtgattgcgccggtggtggcgccg

SEQ ID NO: 471 - DNA, 36 bp, Artificial Sequence
  The polynucleotide sequence that encodes advanced Macromolecule Transduction Domain (aMTD) peptide for Improvement of Cell-Permeability
gcgctggtggcggtgctgccggcggtggcggtgccg

SEQ ID NO: 472 - DNA, 36 bp, Artificial Sequence
  The polynucleotide sequence that encodes advanced Macromolecule Transduction Domain (aMTD) peptide for Improvement of Cell-Permeability
gcgctggtggcgccgctgctggcggtggcggtgccg

SEQ ID NO: 473 - DNA, 36 bp, Artificial Sequence
  The polynucleotide sequence that encodes advanced Macromolecule Transduction Domain (aMTD) peptide for Improvement of Cell-Permeability
gcggtgctggcggtggtggcgccggtggtggcgccg

SEQ ID NO: 474 - DNA, 36 bp, Artificial Sequence
  The polynucleotide sequence that encodes advanced Macromolecule Transduction Domain (aMTD) peptide for Improvement of Cell-Permeability
gcggtgattgcggtggcgccgctggtggtggcgccg

SEQ ID NO: 475 - DNA, 36 bp, Artificial Sequence
  The polynucleotide sequence that encodes advanced Macromolecule Transduction Domain (aMTD) peptide for Improvement of Cell-Permeability
gcggtgattgcgctggcgccggtggtggtggcgccg

SEQ ID NO: 476 - DNA, 36 bp, Artificial Sequence
  The polynucleotide sequence that encodes advanced Macromolecule Transduction Domain (aMTD) peptide for Improvement of Cell-Permeability
gtggcgattgcgctggcgccggtggtggtggcgccg

SEQ ID NO: 477 - DNA, 36 bp, Artificial Sequence
  The polynucleotide sequence that encodes advanced Macromolecule Transduction Domain (aMTD) peptide for Improvement of Cell-Permeability
gtggcgctggcgctggcgccggtggtggtggcgccg

SEQ ID NO: 478 - DNA, 36 bp, Artificial Sequence
  The polynucleotide sequence that encodes advanced Macromolecule Transduction Domain (aMTD) peptide for Improvement of Cell-Permeability
gtggcggcgctgctgccggcggtggtggtggcgccg

SEQ ID NO: 479 - DNA, 36 bp, Artificial Sequence
  The polynucleotide sequence that encodes advanced Macromolecule Transduction Domain (aMTD) peptide for Improvement of Cell-Permeability
gtggcgctggcgctgccggcggtggtggtggcgccg

SEQ ID NO: 480 - DNA, 36 bp, Artificial Sequence
  The polynucleotide sequence that encodes advanced Macromolecule Transduction Domain (aMTD) peptide for Improvement of Cell-Permeability
gtggcgctgctggcgccggcggtggtggtggcgccg

SEQ ID NO: 481 - DNA, 57 bp, Artificial Sequence
  cDNA Sequence of Histidine Tag
atgggcagcagccatcatcatcatcatcacagcagcggcctggtgccgcgcggcagc

SEQ ID NO: 482 - protein, 19 aa, Artificial Sequence
  Amino Acid Sequence of Histidine Tag
MGSSHHHHHHSSGLVPRGS

SEQ ID NO: 483 - DNA, 36 bp, Artificial Sequence; identical to SEQ ID NO: 257
  cDNA Sequences of aMTD61

SEQ ID NO: 484 - protein, 12 aa, Artificial Sequence
  Amino Acid Sequences of aMTD61
VAALPVLLAALP

SEQ ID NO: 485 - DNA, 36 bp, Artificial Sequence
  cDNA Sequences of aMTD165
ccggtggcggtgccggtggcgctggcgattgtgccg

SEQ ID NO: 486 - protein, 12 aa, Artificial Sequence
  Amino Acid Sequences of aMTD165
ALAVPVALAIVP

SEQ ID NO: 487 - DNA, 561 bp, Artificial Sequence
  cDNA Sequence of human GPX7
atggtggcggcgacggtggcagcggcgtggctgctcctgtgggctgcggcctgcgcgcagcaggagcaggacttctacgacttcaaggcggtcaacatccggggcaaactggtgtcgctggagaagtaccgcggatcggtgtccctggtggtgaatgtggccagcgagtgcggcttcacagaccagcactaccgagccctgcagcagctgcagcgagacctgggcccccaccacttcaacgtgctcgccttcccctgcaaccagtttggccaacaggagcctgacagcaacaaggagattgagagctttgcccgccgcacctacagtgtctcattccccatgtttagcaagattgcagtcaccggtactggtgcccatcctgccttcaagtacctggcccagacttctgggaaggagcccacctggaacttctggaagtacctagtagccccagatggaaaggtggtaggggcttgggacccaactgtgtcagtggaggaggtcagaccccagatcacagcgctcgtgaggaagctcatcctactgaagcgagaagactta

SEQ ID NO: 488 - protein, 187 aa, Artificial Sequence
  Amino Acid Sequence of human GPX7
MVAATVAAAWLLLWAAACAQQEQDFYDFKAVNIRGKLVSLEKYRGSVSLVVNVASECGFTDQHYRALQQLQRDLGPHHFNVLAFPCNQFGQQEPDSNKEIESFARRTYSVSFPMFSKIAVTGTGAHPAFKYLAQTSGKEPTWNFWKYLVAPDGKVVGAWDPTVSVEEVRPQITALVRKLILLKREDL

SEQ ID NO: 489 - DNA, 552 bp, Artificial Sequence
  cDNA Sequences of SDA
atggcaaatattaccgttttctataacgaagacttccagggtaagcaggtcgatctgccgcctggcaactatacccgcgcccagttggcggcgctgggcatcgagaataataccatcagctcggtgaaggtgccgcctggcgtgaaggctatcctgtaccagaacgatggtttcgccggcgaccagatcgaagtggtggccaatgccgaggagttgggcccgctgaataataacgtctccagcatccgcgtcatctccgtgcccgtgcagccgcgcatggcaaatattaccgttttctataacgaagacttccagggtaagcaggtcgatctgccgcctggcaactatacccgcgcccagttggcggcgctgggcatcgagaataataccatcagctcggtgaaggtgccgcctggcgtgaaggctatcctctaccagaacgatggtttcgccggcgaccagatcgaagtggtggccaatgccgaggagctgggtccgctgaataataacgtctccagcatccgcgtcatctccgtgccggtgcagccgagg

SEQ ID NO: 490 - protein, 184 aa, Artificial Sequence
  Amino Acid Sequences of SDA
MANITVFYNEDFQGKQVDLPPGNYTRAQLAALGIENNTISSVKVPPGVKAILYQNDGFAGDQIEVVANAEELGPLNNNVSSIRVISVPVQPRMANITVFYNEDFQGKQVDLPPGNYTRAQLAALGIENNTISSVKVPPGVKAILYQNDGFAGDQIEVVANAEELGPLNNNVSSIRVISVPVQPR

SEQ ID NO: 491 - DNA, 297 bp, Artificial Sequence
  cDNA Sequences of SDB
atggcagaacaaagcgacaaggatgtgaagtactacactctggaggagattcagaagcacaaagacagcaagagcacctgggtgatcctacatcataaggtgtacgatctgaccaagtttctcgaagagcatcctggtggggaagaagtcctgggcgagcaagctgggggtgatgctactgagaactttgaggacgtcgggcactctacggatgcacgagaactgtccaaaacatacatcatcggggagctccatccagatgacagatcaaagatagccaagccttcggaaaccctt

SEQ ID NO: 492 - protein, 99 aa, Artificial Sequence
  Amino Acid Sequences of SDB
MAEQSDKDVKYYTLEEIQKHKDSKSTWVILHHKVYDLTKFLEEHPGGEEVLGEQAGGDATENFEDVGHSTDARELSKTYIIGELHPDDRSKIAKPSETL

SEQ ID NO: 493 - DNA, 327 bp, Artificial Sequence
  cDNA Sequences of SDC
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcc

SEQ ID NO: 494 - protein, 109 aa, Artificial Sequence
  Amino Acid Sequences of SDC
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 495 - DNA, 624 bp, Artificial Sequence
  cDNA Sequences of SDD
atgaaaaagatttggctggcgctggctggtttagttttagcgtttagcgcatcggcggcgcagtatgaagatggtaaacagtacactaccctggaaaaaccggtagctggcgcgccgcaagtgctggagtttttctctttcttctgcccgcactgctatcagtttgaagaagttctgcatatttctgataatgtgaagaaaaaactgccggaaggcgtgaagatgactaaataccacgtcaacttcatgggtggtgacctgggcaaagatctgactcaggcatgggctgtggcgatggcgctgggcgtggaagacaaagtgactgttccgctgtttgaaggcgtacagaaaacccagaccattcgttctgcttctgatatccgcgatgtatttatcaacgcaggtattaaaggtgaagagtacgacgcggcgtggaacagcttcgtggtgaaatctctggtcgctcagcaggaaaaagctgcagctgacgtgcaattgcgtggcgttccggcgatgtttgttaacggtaaatatcagctgaatccgcagggtatggataccagcaatatggatgtttttgttcagcagtatgctgatacagtgaaatatctgtccgagaaaaaa

SEQ ID NO: 496 - protein, 208 aa, Artificial Sequence
  Amino Acid Sequences of SDD
MKKIWLALAGLVLAFSASAAQYEDGKQYTTLEKPVAGAPQVLEFFSFFCPHCYQFEEVLHISDNVKKKLPEGVKMTKYHVNFMGGDLGKDLTQAWAVAMALGVEDKVTVPLFEGVQKTQTIRSASDIRDVFINAGIKGEEYDAAWNSFVVKSLVAQQEKAAADVQLRGVPAMFVNGKYQLNPQGMDTSNMDVFVQQYADTVKYLSEKK

SEQ ID NO: 497 - DNA, 300 bp, Artificial Sequence
  cDNA Sequences of SDE
gggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcaccgcgaacagattggaggt

SEQ ID NO: 498 - protein, 100 aa, Artificial Sequence
  Amino Acid Sequences of SDE
GSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGG

SEQ ID NO: 499 - DNA, 890 bp, Artificial Sequence
  cDNA Sequences of SDF
ggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccgg

SEQ ID NO: 500 - protein, 297 aa, Artificial Sequence
  Amino Acid Sequences of SDF
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

(19) United States
(12) Patent Application Publication
(10) Pub. No.: US 2016/0068825 A1
(43) Pub. Date: Mar. 10, 2016

(54) Development of Protein-Based Biotherapeutics That Penetrate Cell-Membrane and Induce Anti-Cancer Effect- Cell-Permeable Glutathione Peroxidase7 (CP-GPX7) in Gastrointestinal Track (GIT), Polynucleotides Encoding the Same, and Anti-Cancer Compositions Comprising the Same

(71) Applicants: DAEWOONG JO, Brentwood, TN (US); Cellivery Therapeutics, Inc., Seoul (KR)

(72) Inventors: Daewoong JO, Brentwood, TN (US); Young Sil CHOI, Seoul (KR); So Yeong LEE, Seoul (KR); Eun Kyung LEE, Seoul (KR); Mi Rin KANG, Seoul (KR)

(21) Appl. No.: 14/846,474

(22) Filed: Sep. 4, 2015

Related U.S. Application Data
(60) Provisional application No. 62/046,067, filed on Sep. 4, 2014.

(51) Int. Cl. C12N 9/08; A61K 38/44

(52) U.S. Cl. CPC C12N 9/0065; A61K 38/44; C12Y 111/01009

(57) ABSTRACT

Gastrointestinal track (GIT) including oesophageal and gastric cancers are a leading cause of cancer death worldwide. Limited therapeutic options highlight the need to understand the molecular changes responsible for the disease and to develop therapies based on this understanding. Advances in understanding the molecular changes responsible for GIT cancer etiology and progression are expected to improve disease diagnosis and treatment. The glutathione peroxidase 7 (GPX7) a candidate tumor suppressor implicated in GIT cancers including esophageal and gastric cancers has been implicated as a potential tumor suppressor gene in esophageal and gastric cancers; however, this claim is controversial. The goal of this invention is to develop cell-permeable (CP-) form of GPX7 to utilize the therapeutic potential of GPX7 in the treatment of GIT cancers. Using macromolecule intracellular transduction technology (MITT) enabled by novel hydrophobic cell-penetrating peptide (CPP) called advanced macromolecule transduction domains (aMTDs) which are able to promote protein uptake by mammalian cells and tissues, the first CP-GPX7 protein has been developed to deliver biologically active GPX7 protein into human oesophageal and gastric cancer cells, resulting in suppression of cell phenotypes and induction of changes in biomarker expression consistent with previously described effects of GPX7. CP-GPX7 recombinant protein fused to aMTD also suppresses the growth of human gastric tumors in a mouse xenograft model. The results of this art provide further evidence that GPX7 can function as an anti-cancer molecule and suggest that practical methods to augment GPX7 function could be useful in treating of some types of GIT cancers. The present art with CP-GPX7 recombinant protein illustrates the use of protein-based therapies to target GIT cancers.

### CROSS-REFERENCE TO RELATED APPLICATIONS

This application claims the benefit of the filing date of U.S. Provisional Application No. 62/046,067, filed on Sep. 4, 2014, in the United States Patent and Trademark Office, the disclosure of which is incorporated herein in its entirety by reference.

### TECHNICAL FIELD

The present invention pertains to have (i) cell-permeable GPX7 (CP-GPX7) proteins as protein-based biotherapeutics, which are well-enhanced in their ability to transport biologically active GPX7 proteins across the plasma membrane, to increase in its solubility and manufacturing yield, and to induce anti-cancer effect in gastrointestinal track (GIT) (ii) polynucleotides that encode the same, and (iii) anti-cancer compositions that comprise the same.

### BACKGROUND ART

Gastrointestinal track (GIT) cancer including gastric and oesophageal cancer is the most common cancer in Asian countries (e.g., Korea, Japan) and a leading cause of cancer death worldwide, provoking considerable effort to understand the pathogenesis of the disease and to develop improved methods for diagnosis and treatment. Therapeutic options are limited for GIT-cancer not cured by surgical resection, and over-all 5-year survival rates are in the range of 30%.

Gastro-oesophageal reflux disease (GORD) is a condition where gastric acid, usually mixed with bile acids, refluxes into the lower oesophagus. GORD-associated chronic mucosal injury and inflammation is a major risk factor for the development of Barrett's oesophagus (BO), a premalignant condition that is closely associated with the development of oesophageal adenocarcinoma (OAC). Patients with BO can progress to low-grade dysplasia (LGD), high-grade dysplasia (HGD), and OAC at 30 to 60 times that of the general population.

Gastric acid and/or in combination with bile acids has been reported to induce a significant increase in intracellular reactive oxygen species (ROS) in oesophageal epithelial cells. The ROS levels are known to be higher in tissues with Barrett's oesophagus and OAC. In addition, higher levels of oxidative DNA damage, as well as single and double strand breaks in human Barrett's oesophagus and OAC are also major characteristics of the GIT-cancers. Normal cells have intact anti-oxidative properties that protect cells from ROS-induced DNA damage.

Among these systems, the glutathione peroxidase (GPX) family is a major anti-oxidative enzyme family that catalyzes the reduction of hydrogen peroxide, organic hydroperoxide, and lipid peroxides by reduced glutathione. It has been recently reported a frequent dysfunction of glutathione peroxidase 7 (GPX7) in OAC and its precancerous BO and dysplasia, suggesting that impairment of the anti-oxidative capacity may contribute to the development of OAC. GPX7 is the first example of a novel class of tumor suppressor genes, which are of particular importance in the proximal digestive tract to limit adenocarcinoma growth. A recent discovery showed that GPX7 deficiency in mice leads to systemic oxidative stress, increased tumor suppressor functions in OAC. Loss of expression and dysfunction of GPX7 are frequent in OAC and its precancerous lesions. GPX7 hypermethylation is observed in cancer and Barrett's oesophagus, but not in adjacent normal squamous epithelium. Methylation of tumor suppressor pathway is emerging as a major anti-cancer mechanism across the entire tract.

In principle, protein-based therapeutics offer to a way to control biochemical processes in living cells under non steady-state conditions and with fewer off target effects than conventional small molecule therapeutics. In practice, systemic protein delivery in animals has proven difficult due to poor tissue penetration and rapid clearance. Protein transduction exploits the ability of some cell-penetrating peptide (CPP) sequences to enhance the uptake of proteins and other macromolecules by mammalian cells. Previously developed hydrophobic CPPs, named membrane translocating sequence (MTS), membrane translocating motif (MTM) and macromolecule transduction domain (MTD), are able to deliver biologically active proteins into a variety of cells and tissues. Various cargo proteins fused to these CPPs have been used to test the functional and/or therapeutic efficacy of protein transduction.

However, the recombinant proteins fused to previously developed hydrophobic CPPs displayed extremely low solubility, poor yields and relatively low cell- and tissue-permeability. Therefore, these recombinant proteins were not suitable for further clinical development as therapeutic agents. To overcome these limitations, cell-permeable GPX7 recombinant proteins (CP-GPX7) fused to the combination of novel hydrophobic CPPs, namely advanced macromolecule transduction domains (aMTDs) to greatly improve the efficiency of membrane penetrating ability in vitro and in vivo with solubilization domains to increase in their solubility and manufacturing yield when expressed and purified from bacteria cells.

In this new art of invention, aMTD/SDs-fused GPX7 recombinant proteins (CP-GPX7), much improved physicochemical characteristics (solubility & yield) and functional activity (cell-/tissue-permeability) compared with the proteins fused to previously developed hydrophobic CPPs. In addition, the newly developed CP-GPX7 has now been demonstrated to have therapeutic application in treating GIT cancer, exploiting the ability of GPX7 to suppress NF-κB signaling. The present invention represents that macromolecule intracellular transduction technology (MITT) enabled by the new hydrophobic CPPs that are aMTD may provide novel protein therapy through GPX7-intracellular protein replacement against the GIT cancer. These findings suggest that intracellular restoration of GPX7 with CP-GPX7 creates a new paradigm for anti-cancer therapy, and the intracellular protein replacement therapy with the GPX7 recombinant protein fused to the combination of aMTD and SDs pair may be useful to treat the cancer.

### SUMMARY

The present invention relates to cell-permeable GPX7 (CP-GPX7) recombinant proteins capable of mediating the transduction of biologically active macromolecules into live cells.

CP-GPX7 fused to novel hydrophobic CPPs—namely advanced macromolecule transduction domains (aMTDs)—greatly improve the efficiency of membrane penetrating ability in vitro and in vivo of the recombinant proteins.

CP-GPX7 fused to solubilization domains (SDs) greatly increase in their solubility and manufacturing yield when they are expressed and purified in the bacteria system.

The present invention also, relates to its therapeutic application for delivery of a biologically active molecule to a cell, involving a cell-permeable GPX7 recombinant protein, where the aMTD is attached to a biologically active cargo molecule.

Other aspects of the present invention relate to an efficient use of aMTD sequences for drug delivery, protein therapy, intracellular protein therapy, protein replacement therapy and peptide therapy.

The present invention provides cell-permeable GPX7 as a biotherapeutics having improved solubility/yield and cell-/tissue-permeability and anti-cancer effects in gastrointestinal track (GIT). Therefore, this would allow their practically effective applications in drug delivery and protein therapy including intracellular protein therapy and protein replacement therapy.

### BRIEF DESCRIPTION OF DRAWINGS

The above and other objects, features and other advantages of the present invention will be more clearly understood from the following detailed description taken in conjunction with the accompanying drawings.

FIG. 1 shows aMTDs-mediated SDA recombinant proteins delivery into cells. Uptake of aMTD fused-protein (HM61SA and HM165SA) by RAW264.7 cells. Cells were exposed to 10 μmol/L of the FITC conjugated proteins containing aMTD (HM61SA and HM165SA, red) or lacking aMTD (HSA, blue) or 10 μmol/L of FITC alone (green) for 1 hour, and analyzed by flow cytometry.

FIG. 2 shows visualized cell-permeability of aMTDs-fused SDA recombinant proteins. aMTD fused-protein uptake by NIH3T3 cells. NIH3T3 cells were incubated with 10 μmol/L unconjugated FITC (FITC only) and FITC-conjugated proteins containing aMTD (HM61SA and HM165SA) for 1 hour, and visualized by fluorescence confocal laser scanning microscopy.

FIG. 3 shows the structure of GPX7 recombinant proteins—Set 1. A schematic diagram of GPX7 recombinant proteins having cell-permeability is presented and constructed according to the present invention. Set 1 of GPX7 recombinant proteins (HG7, HM61G7, HM61G7SA and HM61G7SB) contained histidine tag for affinity purification (MGSSHHHHHHSSLVPRGSH, white), GPX7 (gray), aMTD61 (red), SDA (blue) and SDB (light blue).

FIG. 4 shows the construction of expression vector for GPX7 recombinant proteins—Set 1. Agarose gel electrophoresis analysis show plasmid DNA fragments insert encoding set 1 of aMTD-GPX7-SD recombinant proteins cloned into the pET-28a (+) vector.

FIG. 5 shows the inducible expression and purification of GPX7 recombinant proteins—Set 1. Set 1 of GPX7 recombinant proteins were expressed in E. coli BL21-Gold (DE3). SDS-PAGE analysis of cell lysates before (−) and after (+) IPTG induction; aliquots of Ni2+ affinity purified proteins (P); and molecular weight standard (M). The yield (mg/L) and solubility of each recombinant protein is indicated. Solubility was scored on a 5-point scale from highly soluble, with little tendency to precipitate (+++++), to largely insoluble proteins (+).

FIG. 6 shows the structural change of GPX7 recombinant proteins with aMTD/SD-fusion—Set 2. A schematic diagram of GPX7 recombinant proteins having cell-permeability is presented and constructed according to the present invention. Set 2 of GPX7 recombinant proteins (SCHG7M165, SCHM165 and SCHG7) contained histidine tag for affinity purification (white), GPX7 (gray), aMTD165 (red) and SDC (yellow).

FIG. 7 shows the construction of expression vector for GPX7 recombinant proteins—Set 2. Agarose gel electrophoresis analysis show plasmid DNA fragments insert encoding set 2 of aMTD-GPX7-SD recombinant proteins cloned into the pET-32a (+) vector.

FIG. 8 shows the inducible expression and purification of GPX7 recombinant proteins—Set 2. Set 2 of GPX7 recombinant proteins were expressed in E. coli BL21-Gold (DE3). SDS-PAGE analysis of cell lysates before (−) and after (+) IPTG induction; aliquots of Ni2+ affinity purified proteins (P); and molecular weight standard (M). The yield (mg/L) and solubility of each recombinant protein is indicated. Solubility was scored on a 5-point scale from highly soluble, with little tendency to precipitate (+++++), to largely insoluble proteins (+).

FIG. 9 shows the structural change of GPX7 recombinant proteins with aMTD/SD-fusion—Set 3. A schematic diagram of GPX7 recombinant proteins having cell-permeability is presented and constructed according to the present invention. Set 3 of GPX7 recombinant proteins (SDHG7M165 and M165G7SFH) contained histidine tag for affinity purification (white), GPX7 (gray), aMTD165 (red), SDD (orange) and SDF (scarlet).

FIG. 10 shows the construction of expression vectors for GPX7 recombinant proteins—Set 3. Agarose gel electrophoresis analysis show plasmid DNA fragments insert encoding set 3 of aMTD-GPX7-SD recombinant proteins cloned into the pET-39b (+) vector and pH6HTC His6HaloTag®T7 vector.

FIG. 11 shows the inducible expression and purification of GPX7 recombinant proteins—Set 3. Set 3 of GPX7 recombinant proteins were expressed in E. coli BL21-Gold (DE3). SDS-PAGE analysis of cell lysates before (−) and after (+) IPTG induction; aliquots of Ni2+ affinity purified proteins (P); and molecular weight standard (M). The yield (mg/L) and solubility of each recombinant protein is indicated. Solubility was scored on a 5-point scale from highly soluble, with little tendency to precipitate (+++++), to largely insoluble proteins (+).

FIG. 12 shows the structure of aMTD/SD-fused GPX7 recombinant proteins—Final Clones. A schematic diagram of GPX7 recombinant proteins having cell-permeability is presented and constructed according to the present invention. Final clones of GPX7 recombinant proteins (HSAM165G7SB, HSAG7SB, HSAM165SB) contained histidine tag for affinity purification (white), GPX7 (gray), aMTD165 (red), SDA (blue) and SDB (light blue).

FIG. 13 shows the construction of expression vectors for GPX7 recombinant proteins—Final Clones. Agarose gel electrophoresis analysis show plasmid DNA fragments insert encoding final clones of aMTD-GPX7-SD recombinant proteins cloned into the pET-28a (+) vector.

FIG. 14 shows the expression, purification and determination of solubility/yield of SD-fused GPX7 recombinant proteins. Final clones of GPX7 recombinant proteins were expressed in E. coli BL21-Gold (DE3). SDS-PAGE analysis of cell lysates before (−) and after (+) IPTG induction; aliquots of Ni2+ affinity purified proteins (P); and molecular weight standard (M). The yield (mg/L) and solubility of each recombinant protein is indicated. Solubility was scored on a 5-point scale from highly soluble, with little tendency to precipitate (+++++), to largely insoluble proteins (+).

FIG. 15 shows the aMTD-mediated cell-permeability of GPX7 Recombinant Proteins. Uptake of aMTD-GPX7 protein (HSAM165G7SB) by RAW264.7 cells. Cells were exposed to 10 μmol/L of the FITC conjugated GPX7 recombinant proteins containing aMTD (HSAM165G7SB, red) or lacking aMTD (HSAG7SB, blue) or 10 μmol/L of FITC alone (green) for 1 hour, and analyzed by flow cytometry.

FIG. 16 aMTD-Mediated Intracellular Delivery and Localization of GPX7 Recombinant Proteins. GPX7 recombinant protein uptake by NIH3T3 cells. NIH3T3 cells were incubated with 10 μmol/L unconjugated FITC (FITC only) and FITC-conjugated recombinant aMTD-GPX7 proteins for 1 hours, and visualized by fluorescence confocal laser scanning microscopy.

FIG. 17 Systemic Delivery of aMTD/SD-Fused GPX7 Recombinant Proteins In Vivo. Systemic GPX7 recombinant protein delivery to murine tissues. Cryosections (20 μm) of organs were prepared from mice 2 hours after intraperitoneal injection of 75 μg FITC or FITC-labeled GPX7 proteins with (HSAM165G7SB) and without (HSAG7SB) the aMTD165 sequence. Tissue distribution of the recombinant proteins (green staining) was assessed by fluorescence microscopy.

FIG. 18 Mechanism of aMTD-Mediated GPX7 Recombinant Proteins Uptake into Cells. (A) Cell surface protein-independence of aMTD165-mediated protein uptake. (B) Endocytosis-independence of aMTD165 mediated protein uptake. (C) ATP source-independence of aMTD165-mediated protein uptake. (D) EDTA suppresses aMTD165-mediated protein uptake. (E) Temperature-dependence of aMTD165-stimulated protein uptake. Cells (shaded) were exposed for one hour to HSAM165G7SB (red), HSAG7SB (blue) and FITC (green) were processed as before to remove non-internalized protein and were analyzed by flow cytometry.

FIG. 19 Inhibition of TNF-α-Mediated NF-κB Nuclear Translocation in Human Gastric Cancer Cells with CP-GPX7. Immunofluorescence staining of NF-κB-p65 in human gastric cancer cells (AGS and MKN45). Cells were treated with TNF-α (50 ng/ml) for 30 minutes alone or in combination with CP-CPX7 for 2 hours. Control cells treated with TNF-α were increased p65 nuclear staining in nucleus. However, cells treated with TNF-α and HSAM165G7SB were decreased TNF-α-induced p65 nuclear staining.

FIG. 20 Suppression of NF-κB Phosphorylation Induced by TNF- [symbol] [symbol] in Human Gastric Cancer Cells with CP-GPX7. Western blot analysis of p65 protein in human gastric cancer cells. Human gastric cancer cells (AGS and MKN74) were treated with GPX7 recombinant proteins of 10 μM for 24 hours. HSAM165G7SB suppressed phosphorylation of p65 in gastric cancer cells.

FIG. 21 Cell-Permeability of CP-GPX7 (HSAM165G7SB) in Gastric Cancer Cells. Systemic GPX7 recombinant protein delivery to murine stomach. Cryosections (20 μm) of organs were prepared from mice 2 hours after intraperitoneal injection of 75 μg FITC or FITC-labeled GPX7 proteins with (HSAM165G7SB) and without (HSAG7SB) the aMTD165 sequence. Tissue distribution of the recombinant proteins (green staining) was assessed by fluorescence microscopy.

FIG. 22 Tissue Distribution of CP-GPX7 (HSAM165G7SB) into Stomach. Uptake of aMTD-GPX7 protein (HSAM165G7SB) by gastric cancer cells (AGS and MKN75). Cells were exposed to 10 μmol/L of the FITC conjugated GPX7 recombinant proteins containing aMTD (HSAM165G7SB, red) or lacking aMTD (HSAG7SB, blue) or 10 μmol/L of FITC alone (green) for 1 hour, and analyzed by flow cytometry.

FIG. 23 Inhibition of Human Gastric Cancer Cell Proliferation with CP-GPX7. Human gastric cancer cells (MKN45, AGS, MKN75 and MKN74) were incubated with either no treatment (medium) as a negative control or CP-GPX7 at 1, 5, and 10 μmol/L for 72 hours. Cell viability was then determined using the Cell-Titer Glo assay. Bar chart shows percentage cell viability relative to the medium control. Treatment with CP-GPX7 significantly reduced gastric cancer cell viability (*p<0.05) in a time-dependent manner. This figure is representative of at least three independent experiments.

FIG. 24 Inhibition of Human Oesophageal Cancer Cell Proliferation with CP-GPX7. Human oesopageal cancer cells (FLO-1, OE19 and OE33) were treated with HSAG7SB or HSAM165SB or HSAM165G7SB proteins 10 μmol/L for 72 hours. Cell viability was then determined using the Cell-Titer Glo assay. Bar chart shows percentage cell viability relative to the medium control. Treatment with CP-GPX7 significantly reduced gastric cancer cell viability (*p<0.05) in a time-dependent manner. This figure is representative of at least three independent experiments.

FIG. 25 Regulation of Tumor Suppressor Genes in Human Gastric Cancer Cells with CP-GPX7.

CP-GPX7 suppressed phosphorylation of RB, enhanced the expression of the p21 in gastric cancer cells. Gastric cancer cells (AGS and MKN74) were treated with the indicated 10 μM proteins for 24 hours and cell extracts were immunoblotted with antibody against p21, phospho-retinoblastoma tumor suppressor (RB) and β-actin.

FIG. 26 Stimulation of Apoptosis in Human GIT-Cancer Cell with CP-GPX7 Recombinant Proteins. Apoptotic GIT-cancer cells were determined by TUNEL staining and visualized at 200× magnification. Red color is TUNEL staining representing apoptotic cell; blue color is the cell nucleus stained by DAPI.

FIG. 27 Stimulation of Apoptosis in Gastric Cancer Cells with CP-GPX7. Human Gastric cancer cells, AGS, were treated for 24 hrs with 10 μM HSAM165G7SB, HSAG7SB or HSAM165SB proteins and analyzed by flow cytometry of cells stained with annexin-V and 7-AAD.

FIG. 28 Inhibition of Human GIT-Cancer Cell Migration with CP-GPX7. Gastric cancer cells, AGS and MKN75, and oesophageal cancer cells, SK-GT-4, were treated with HSAM165G7SB or HSAG7SB proteins for 2 hour in serum-free media, visualized after an additional 24 hours or 72 hours in serum-free media. Photographed data shown here are representative of 3 independent assays. The data are presented as means±SD. *P<0.05 as determined by a Student unpaired t test.

FIG. 29 Inhibition of Migration/Invasion in Human GIT-Cancer Cells with CP-GPX7. Gastric cancer cells, AGS, and oesophageal cancer cells, FLO-1, were treated with HSAM165G7SB or HSAM165SB proteins for 24 hour in serum-free media, and migration/invasion were measured by Transwell assay. The data are presented as means±SD. **P<0.01 as determined by a Student unpaired t test.

FIG. 30 External Appearances of Gastric Tumor Bearing Mice

Female Balb/c nu/nu mice were implanted with NCI-N87 tumor block (1 mm3) into left side of back. After tumor reached a size of 50-80 mm3 (start), the mice were injected daily (I.V.) for 3 weeks with the diluent alone, HSAG7SB or HSAM165G7SB (CP-GPX7) and observed for 2 weeks following the termination of the treatment.

FIG. 31 Suppression of Subcutaneously Implanted Gastric Cancer with CP-GPX7 Female Balb/c nu/nu mice were subcutaneously implanted with NCI-N87 tumor block (1 mm3) and after tumor reached size of 50-80 mm3 (start), the mice were injected daily (I.V.) for 3 weeks with the diluent alone, HSAG7SB or HSAM165G7SB (CP-GPX7) and observed for 2 weeks following the termination of the treatment. Tumor volumes were measured in the indicated day.

### DETAILED DESCRIPTION

In this invention, it has been hypothesized that exogenously administered GPX7 proteins could compensate for the apparent inability of endogenously expressed members of this physiologic regulator to interrupt constitutively active cancer-initiating NF-κB signaling and excessive cell cycle, resulting in the inhibition of the tumorigenesis. To prove our hypothesis, the GPX7 recombinant proteins were fused to novel hydrophobic CPPs called aMTDs to have high cell-/tissue-permeability, additionally adopted solubilization domains to increase their solubility/yield in physiological condition, and then tested whether exogenous administration of GPX7 proteins can reconstitute their endogenous stores and restore their basic function as the inhibitor that attenuates NF-κB signaling. This art of invention has demonstrated “intracellular protein therapy” by designing and introducing cell-permeable form of GPX7 that has a great potential of anti-cancer therapeutic applicability in gastrointestinal track.

### 1. Novel Hydrophobic Cell-Penetrating Peptides—Advanced Macromolecule Transduction Domains

1-1. Analysis of Previously Developed Hydrophobic CPPs

To address the limitation of previously developed hydrophobic CPPs, novel sequences have been developed. To design new hydrophobic CPPs for intracellular delivery of cargo proteins such as GPX7, identification of optimal common sequence and/or homologous structural determinants, namely critical factors (CFs), had been crucial. To do it, the physicochemical characteristics of previously published hydrophobic CPPs were analyzed. To keep the similar mechanism on cellular uptake, all CPPs analyzed were hydrophobic region of signal peptide (HRSP)-derived CPPs (e.g. membrane translocating sequence: MTS and macromolecule transduction domain: MTD) as explained previously.

### (1) Basic Characteristics of CPPs Sequence.

These 17 hydrophobic CPPs published from 1995 to 2014 have been analyzed for their 11 different characteristics—sequence, amino acid length, molecular weight, pI value, bending potential, rigidity/flexibility, structural feature, hydropathy, residue structure, amino acid composition, and secondary structure of the sequences. Two peptide/protein analysis programs were used (ExPasy: http://web.expasy.org/protparam/, SoSui: http://harrier.nagahama-i-bio.ac.jp/sosui/sosui_submit.html) to determine various indexes, structural features of the peptide sequences and to design new sequence. Followings are important factors analyzed.

Average length, molecular weight and pI value of the peptides analyzed were 10.8±2.4, 1,011±189.6 and 5.6±0.1, respectively.

### (2) Bending Potential (Proline Position: PP)

Bending potential (Bending or No-Bending) was determined based on the fact whether proline (P) exists and/or where the amino acid(s) providing bending potential to the peptide in recombinant protein is/are located. Proline differs from the other common amino acids in that its side chain is bonded to the backbone nitrogen atom as well as the alpha-carbon atom. The resulting cyclic structure markedly influences protein architecture which is often found in the bends of folded peptide/protein chain. Eleven out of 17 were determined as ‘Bending’ peptide which means that proline should be present in the middle of sequence for peptide bending and/or located at the end of the peptide for protein bending. As indicated above, peptide sequences could penetrate the plasma membrane in a “bent” configuration. Therefore, bending or no-bending potential is considered as one of the critical factors for the improvement of current hydrophobic CPPs.

### (3) Rigidity/Flexibility (Instability Index: II)

Since one of the crucial structural features of any peptide is based on the fact whether the motif is rigid or flexible, which is an intact physicochemical characteristic of the peptide sequence, instability index (II) of the sequence was determined. The index value representing rigidity/flexibility of the peptide was extremely varied (8.9-79.1), but average value was 40.1±21.9 which suggested that the peptide should be somehow flexible, but not too rigid or flexible.

### (4) Hydropathy (Grand Average of Hydropathy: GRAVY) and Structural Feature (Aliphatic Index: AI)

Alanine (V), valine (V), leucine (L) and isoleucine (I) contain aliphatic side chain and are hydrophobic—that is, they have an aversion to water and like to cluster. These amino acids having hydrophobicity and aliphatic residue enable them to pack together to form compact structure with few holes. Analyzed peptide sequence showed that all composing amino acids were hydrophobic (A, V, L and I) except glycine (G) in only one out of 17 and aliphatic (A, V, L, I, and P). Their hydropathic index (Grand Average of Hydropathy: GRAVY) and aliphatic index (AI) were 2.5±0.4 and 217.9±43.6, respectively.

### (5) Determination of Critical Factors (CFs)

In the 11 characteristics analyzed, the following 6 are selected namely “Critical Factors (CFs)” for the development of new hydrophobic CPPs—advanced MTDs: i) amino acid length, ii) bending potential (proline presence and location), iii) rigidity/flexibility (instability index: II), iv) structural feature (aliphatic index: AI), v) hydropathy (GRAVY) and vi) amino acid composition/residue structure (hydrophobic and aliphatic A/a).

1-2. Analysis of Selected Hydrophobic CPPs to Optimize ‘Critical Factors’

Since the analyzed data of the 17 different hydrophobic CPPs (analysis A) previously developed during the past 2 decades showed high variation and were hard to make common- or consensus-features, additional analysis B and C was also conducted to optimize the critical factors for better design of improved CPPs-aMTDs.

In analysis B, 8 CPPs were used with each cargo in vivo. Length was 11±3.2, but 3 out of 8 CPPs possessed little bending potential. Rigidity/Flexibility was 41±15, but removing one [MTD85: rigid, with minimal (II: 9.1)] of the peptides increased the overall instability index to 45.6±9.3. This suggested that higher flexibility (40 or higher II) is potentially be better. All other characteristics of the 8 CPPs were similar to the analysis A, including structural feature and hydropathy.

To optimize the ‘Common Range and/or Consensus Feature of Critical Factor’ for the practical design of aMTDs and the random peptides (rPs or rPeptides), which were to prove that the ‘Critical Factors’ determined in the analysis A, B and C were correct to improve the current problems of hydrophobic CPPs—protein aggregation, low solubility/yield, and poor cell/tissue-permeability of the recombinant proteins fused to the MTS/MTM or MTD, and non-common sequence and non-homologous structure of the peptides, empirically selected peptides were analyzed for their structural features and physicochemical factor indexes.

The peptides which did not have a bending potential, rigid or too flexible sequences (too low or too high Instability Index), or too low or too high hydrophobic CPP were unselected, but secondary structure was not considered because helix structure of sequence was not required. 8 selected CPP sequences that could provide a bending potential and higher flexibility were finally analyzed. Common amino acid length is 12 (11.6±3.0). Proline should be presence in the middle of and/or the end of sequence. Rigidity/Flexibility (II) is 45.5-57.3 (Avg: 50.1±3.6). AI and GRAVY representing structural feature and hydrophobicity of the peptide are 204.7±37.5 and 2.4±0.3, respectively. All peptides are consisted with hydrophobic and aliphatic amino acids (A, V, L, I, and P). Therefore, analysis C was chosen as a standard for the new design of new hydrophobic CPPs (TABLE 1).
a. Amino Acid Length: 9-13
b. Bending Potential (Proline Position: PP)
Proline presences in the middle (from 5′ to 8′ amino acid) and at the end of sequence
c. Rigidity/Flexibility (Instability Index: II): 40-60
d. Structural Feature (Aliphatic Index: AI): 180-220
e. Hydropathy (GRAVY): 2.1-2.6
f. Amino Acid Composition: Hydrophobic and Aliphatic amino acids—A, V, L, I and P

TABLE 1
[Universal Structure of Newly Develop Hydrophobic CPPs]
Summarized Critical Factors of aMTD

| | Newly Designed CPPs |
|---|---|
| Critical Factor | Range |
| Bending Potential | Proline presences in the middle (5′, 6′, 7′ or 8′) |
| (Proline Position: PP) | and at the end (12′) of peptides |
| Rigidity/Flexibility | 40-60 |
| (Instability Index: II) | |
| Structural Feature | 180-220 |
| (Aliphatic Index: AI) | |
| Hydropathy | 2.1-2.6 |
| (Grand Average of | |
| Hydropathy GRAVY) | |
| Length | 9-13 |
| (Number of Amino Acid) | |
| Ammo acid Composition | A, V, I, L, P |

1-3. Determination of Critical Factors for Development of aMTDs

For confirming the validity of 6 critical factors providing the optimized cell-/tissue-permeability. all 240 aMTD sequences have been designed and developed based on six critical factors (TABLES 2-1 to 2-6). All 240 aMTDs (hydrophobic, flexible, bending, aliphatic and helical 12 a/a-length peptides are practically confirmed by their quantitative and visual cell-permeability. To determine the cell-permeability of aMTDs and rPeptides which do not satisfy one or more critical factors have also been designed and tested. Relative cell-permeability of 240 aMTDs to the negative control (random peptide (rP) 38, hydrophilic & non-aliphatic 12A/a length peptide) was significantly increased by up to 164 fold, with average increase of 19.6±1.6. Moreover, compared to reference CPPs (MTS/MTM1 and MTD), novel 240 aMTDs averaged of 13±1.1 (maximum 109.9) and 6.6±0.5 (maximum 55.5) fold higher cell-permeability, respectively. As a result, there were vivid association of cell-permeability of the peptides and critical factors. According to the result from the newly designed and tested novel 240 aMTDs, the empirically optimized critical factor (CFs) are provided below (TABLE 3).
a. Amino Acid Length: 12
b. Bending Potential (Proline Position: PP)
Proline presences in the middle (from 5′ to 8′ amino acid) and at the end of sequence
c. Rigidity/Flexibility (Instability Index: II): 41.3-57.3
d. Structural Feature (Aliphatic Index: AI): 187.5-220.0
e. Hydropathy (GRAVY): 2.2-2.6
f. Amino Acid Composition: Hydrophobic and Aliphatic amino acids—A, V, L, I and P

TABLE 2-1
[Newly Developed Hydrophobic CPPs-Amino Acid Sequences of 240 aMTD5 That
All Critical Factors Are Considered and Satisfied (Sequence ID No 1-46)]

| Sequence | | | | Rigidity/ | Sturctural | | |
|---|---|---|---|---|---|---|---|
| ID | | | | Flexibility | Feature | Hydropathy | Residue |
| Number | aMTD | Sequences | Length | (II) | (AI) | (GRAVY) | Structure |
| 1 | 1 | AAALAPVVLALP | 12 | 57.3 | 187.5 | 2.1 | Aliphatic |
| 2 | 2 | AAAVPLLAVVVP | 12 | 41.3 | 195.0 | 2.4 | Aliphatic |
| 3 | 3 | AALLVPAAVLAP | 12 | 57.3 | 187.5 | 2.1 | Aliphatic |
| 4 | 4 | ALALLPVAALAP | 12 | 57.3 | 195.8 | 2.1 | Aliphatic |
| 5 | 5 | AAALLPVALVAP | 12 | 57.3 | 187.5 | 2.1 | Aliphatic |
| 6 | 11 | LLAAVPAVLLAP | 12 | 57.3 | 187.5 | 2.1 | Aliphatic |
| 7 | 12 | LLAAVPAVLLAP | 12 | 57.3 | 211.7 | 2.3 | Aliphatic |
| 8 | 13 | AAALVPVVALLP | 12 | 57.3 | 203.3 | 2.3 | Aliphatic |
| 9 | 21 | AVALLPALLAVP | 12 | 57.3 | 211.7 | 2.3 | Aliphatic |
| 10 | 22 | AVVLVPVLAAAP | 12 | 57.3 | 195.0 | 2.4 | Aliphatic |
| 11 | 23 | VVLVLPAAAAVP | 12 | 57.3 | 195.0 | 2.4 | Aliphatic |
| 12 | 24 | IALAAPALIVAP | 12 | 50.2 | 195.8 | 2.2 | Aliphatic |
| 13 | 25 | IVAVAPALVALP | 12 | 50.2 | 203.3 | 2.4 | Aliphatic |
| 14 | 42 | VAALPVVAVVAP | 12 | 57.3 | 186.7 | 2.4 | Aliphatic |
| 15 | 43 | LLAAPLVVAAVP | 12 | 41.3 | 187.5 | 2.1 | Aliphatic |
| 16 | 44 | ALAVPVALLVAP | 12 | 57.3 | 203.3 | 2.3 | Aliphatic |
| 17 | 61 | VAALPVLLAALP | 12 | 57.3 | 211.7 | 2.3 | Aliphatic |
| 18 | 62 | VALLAPVALAVP | 12 | 57.3 | 203.3 | 2.3 | Aliphatic |
| 19 | 63 | AALLVPALVAVP | 12 | 57.3 | 203.3 | 2.3 | Aliphatic |
| 20 | 64 | AIVALPVAVLAP | 12 | 50.2 | 203.3 | 2.4 | Aliphatic |
| 21 | 65 | IAIVAPVVALAP | 12 | 50.2 | 203.3 | 2.4 | Aliphatic |
| 22 | 81 | AALLPALAALLP | 12 | 57.3 | 204.2 | 2.1 | Aliphatic |
| 23 | 82 | AVVLAPVAAVLP | 12 | 57.3 | 195.0 | 2.4 | Aliphatic |
| 24 | 83 | LAVAAPLALALP | 12 | 41.3 | 195.8 | 2.1 | Aliphatic |
| 25 | 84 | AAVAAPLLLALP | 12 | 41.3 | 195.8 | 2.1 | Aliphatic |
| 26 | 85 | LLVLPAAALAAP | 12 | 57.3 | 195.8 | 2.1 | Aliphatic |
| 27 | 101 | LVALAPVAAVLP | 12 | 57.3 | 203.3 | 2.3 | Aliphatic |
| 28 | 102 | LALAPAALALLP | 12 | 57.3 | 204.2 | 2.1 | Aliphatic |
| 29 | 103 | ALIAAPILALAP | 12 | 57.3 | 204.2 | 2.2 | Aliphatic |
| 30 | 104 | AVVAAPLVLALP | 12 | 41.3 | 203.3 | 2.3 | Aliphatic |
| 31 | 105 | LLALAPAALLAP | 12 | 57.3 | 204.1 | 2.1 | Aliphatic |
| 32 | 121 | AIVALPALALAP | 12 | 50.2 | 195.8 | 2.2 | Aliphatic |
| 33 | 123 | AAIIVPAALLAP | 12 | 50.2 | 195.8 | 2.2 | Aliphatic |
| 34 | 124 | IAVALPALIAAP | 12 | 50.3 | 195.8 | 2.2 | Aliphatic |
| 35 | 141 | AVIVLPALAVAP | 12 | 50.2 | 203.3 | 2.4 | Aliphatic |
| 36 | 143 | AVLALPAVLVAP | 12 | 57.3 | 195.0 | 2.4 | Aliphatic |
| 37 | 144 | VLAIVPAVALAP | 12 | 50.2 | 203.3 | 2.4 | Aliphatic |
| 38 | 145 | LLAVVPAVALAP | 12 | 57.3 | 203.3 | 2.3 | Aliphatic |
| 39 | 161 | AVIALPALIAAP | 12 | 57.3 | 195.8 | 2.2 | Aliphatic |
| 40 | 162 | AVVALPAALIVP | 12 | 50.2 | 203.3 | 2.4 | Aliphatic |
| 41 | 163 | LALVLPAALAAP | 12 | 57.3 | 195.8 | 2.1 | Aliphatic |
| 42 | 164 | LAAVLPALLAAP | 12 | 57.3 | 195.8 | 2.1 | Aliphatic |
| 43 | 165 | ALAVPVALAIVP | 12 | 50.2 | 203.3 | 2.4 | Aliphatic |
| 44 | 182 | ALIAPVVALVAP | 12 | 57.3 | 203.3 | 2.4 | Aliphatic |
| 45 | 183 | LLAAPVVIALAP | 12 | 57.3 | 211.6 | 2.4 | Aliphatic |
| 46 | 184 | LAAIVPAIIAVP | 12 | 50.2 | 211.6 | 2.4 | Aliphatic |

TABLE 2-2
[Newly Developed Hydrophobic CPPs-240 aMTDs That All Critical Factors Are
Considered and Satisfied (Sequence ID No 47-92)]

| Sequence | | | | Rigidity/ | Sturctural | | |
|---|---|---|---|---|---|---|---|
| ID | | | | Flexibility | Feature | Hydropathy | Residue |
| Number | aMTD | Sequences | Length | (II) | (AI) | (GRAVY) | Structure |
| 47 | 185 | AALVLPLIIAAP | 12 | 41.3 | 220.0 | 2.4 | Aliphatic |
| 48 | 201 | LALAVPALAALP | 12 | 57.3 | 195.8 | 2.1 | Aliphatic |
| 49 | 204 | LIAALPAVAALP | 12 | 57.3 | 195.8 | 2.2 | Aliphatic |
| 50 | 205 | ALALVPAIAALP | 12 | 57.3 | 195.8 | 2.2 | Aliphatic |
| 51 | 221 | AAILAPIVALAP | 12 | 50.2 | 195.8 | 2.2 | Aliphatic |
| 52 | 222 | ALLIAPAAVIAP | 12 | 57.3 | 195.8 | 2.2 | Aliphatic |
| 53 | 223 | AILAVPIAVVAP | 12 | 57.3 | 203.3 | 2.4 | Aliphatic |
| 54 | 224 | ILAAVPIALAAP | 12 | 57.3 | 195.8 | 2.2 | Aliphatic |
| 55 | 225 | VAALLPAAAVLP | 12 | 57.3 | 187.5 | 2.1 | Aliphatic |
| 56 | 241 | AAAVVPVLLVAP | 12 | 57.3 | 195.0 | 2.4 | Aliphatic |
| 57 | 242 | AALLVPALVAAP | 12 | 57.3 | 187.5 | 2.1 | Aliphatic |
| 58 | 243 | AAVLLPVALAAP | 12 | 57.3 | 187.5 | 2.1 | Aliphatic |
| 59 | 245 | AAALAPVLALVP | 12 | 57.3 | 187.5 | 2.1 | Aliphatic |
| 60 | 261 | LVLVPLLAAAAP | 12 | 41.3 | 211.6 | 2.3 | Aliphatic |
| 61 | 262 | ALIAVPAIIVAP | 12 | 50.2 | 211.6 | 2.4 | Aliphatic |
| 62 | 263 | ALAVIPAAAILP | 12 | 54.9 | 195.8 | 2.2 | Aliphatic |
| 63 | 264 | LAAAPVVIVIAP | 12 | 50.2 | 203.3 | 2.4 | Aliphatic |
| 64 | 265 | VLAIAPLLAAVP | 12 | 41.3 | 211.6 | 2.3 | Aliphatic |
| 65 | 281 | ALIVLPAAVAVP | 12 | 50.2 | 203.3 | 2.4 | Aliphatic |
| 66 | 282 | VLALVPALIVAP | 12 | 50.2 | 203.3 | 2.4 | Aliphatic |
| 67 | 283 | AALLAPALIVAP | 12 | 50.2 | 195.8 | 2.2 | Aliphatic |
| 68 | 284 | ALIAPAVALIVP | 12 | 50.2 | 211.7 | 2.4 | Aliphatic |
| 69 | 285 | AIVLLPAAVVAP | 12 | 50.2 | 203.3 | 2.4 | Aliphatic |
| 70 | 301 | VIAAPVLAVLAP | 12 | 57.3 | 203.3 | 2.4 | Aliphatic |
| 71 | 302 | LALAPALALLAP | 12 | 57.3 | 204.2 | 2.1 | Aliphatic |
| 72 | 304 | AIILAPIAAIAP | 12 | 57.3 | 204.2 | 2.3 | Aliphatic |
| 73 | 305 | IALAAPILLAAP | 12 | 57.3 | 204.2 | 2.2 | Aliphatic |
| 74 | 321 | IVAVALPALAVP | 12 | 50.2 | 203.3 | 2.3 | Aliphatic |
| 75 | 322 | VVAIVLPALAAP | 12 | 50.2 | 203.3 | 2.3 | Aliphatic |
| 76 | 323 | IVAVALPVALAP | 12 | 50.2 | 203.3 | 2.3 | Aliphatic |
| 77 | 324 | IVAVALPAALVP | 12 | 50.2 | 203.3 | 2.3 | Aliphatic |
| 78 | 325 | IVAVALPAVALP | 12 | 50.2 | 203.3 | 2.3 | Aliphatic |
| 79 | 341 | IVAVALPAVLAP | 12 | 50.2 | 203.3 | 2.3 | Aliphatic |
| 80 | 342 | VIVALAPAVLAP | 12 | 50.2 | 203.3 | 2.3 | Aliphatic |
| 81 | 343 | IVAVALPALVAP | 12 | 50.2 | 203.3 | 2.3 | Aliphatic |
| 82 | 345 | ALLIVAPVAVAP | 12 | 50.2 | 203.3 | 2.3 | Aliphatic |
| 83 | 361 | AVVIVAPAVIAP | 12 | 50.2 | 195.0 | 2.4 | Aliphatic |
| 84 | 363 | AVLAVAPALIVP | 12 | 50.2 | 203.3 | 2.3 | Aliphatic |
| 85 | 364 | LVAAVAPALIVP | 12 | 50.2 | 203.3 | 2.3 | Aliphatic |
| 86 | 365 | AVIVVAPALLAP | 12 | 50.2 | 203.3 | 2.3 | Aliphatic |
| 87 | 381 | VVAIVLPAVAAP | 12 | 50.2 | 195.0 | 2.4 | Aliphatic |
| 88 | 382 | AAALVIPAILAP | 12 | 54.9 | 195.8 | 2.2 | Aliphatic |
| 89 | 383 | VIVALAPALLAP | 12 | 50.2 | 211.6 | 2.3 | Aliphatic |
| 90 | 384 | VIVAIAPALLAP | 12 | 50.2 | 211.6 | 2.4 | Aliphatic |
| 91 | 385 | IVAIAVPALVAP | 12 | 50.2 | 203.3 | 2.4 | Aliphatic |
| 92 | 401 | AALAVIPAAILP | 12 | 54.9 | 195.8 | 2.2 | Aliphatic |

TABLE 2-3
[Newly Developed Hydrophobic CPPs-240 aMTDs That All Critical Factors Are
Considered and Satisfied (Sequence ID No 93-138)]

| Sequence | | | | Rigidity/ | Sturctural | | |
|---|---|---|---|---|---|---|---|
| ID | | | | Flexibility | Feature | Hydropathy | Residue |
| Number | aMTD | Sequences | Length | (II) | (AI) | (GRAVY) | Structure |
| 93 | 402 | ALAAVIPAAILP | 12 | 54.9 | 195.8 | 2.2 | Aliphatic |
| 94 | 403 | AAALVIPAAILP | 12 | 54.9 | 195.8 | 2.2 | Aliphatic |
| 95 | 404 | LAAAVIPAAILP | 12 | 54.9 | 195.8 | 2.2 | Aliphatic |
| 96 | 405 | LAAAVIPVAILP | 12 | 54.9 | 211.7 | 2.4 | Aliphatic |
| 97 | 421 | AAILAAPLIAVP | 12 | 57.3 | 195.8 | 2.2 | Aliphatic |
| 98 | 422 | AVVVAAPVLALP | 12 | 57.3 | 211.7 | 2.4 | Aliphatic |
| 99 | 424 | AVVIAIPVLALP | 12 | 57.3 | 195.0 | 2.4 | Aliphatic |
| 100 | 425 | ALAALVPAVLVP | 12 | 57.3 | 203.3 | 2.4 | Aliphatic |
| 101 | 442 | ALAALVPAVLVP | 12 | 57.3 | 203.3 | 2.3 | Aliphatic |
| 102 | 446 | ALAALVPVALVP | 12 | 57.3 | 203.3 | 2.3 | Aliphatic |
| 103 | 444 | LAAALVPVALVP | 12 | 57.3 | 203.3 | 2.3 | Aliphatic |
| 104 | 445 | ALAALVPALVVP | 12 | 57.3 | 203.3 | 2.3 | Aliphatic |
| 105 | 461 | IAAVIVPAVALP | 12 | 50.2 | 203.3 | 2.4 | Aliphatic |
| 106 | 462 | IAAVLVPAVALP | 12 | 57.3 | 203.3 | 2.4 | Aliphatic |
| 107 | 463 | AVAILVPLLAAP | 12 | 57.3 | 211.7 | 2.4 | Aliphatic |
| 108 | 464 | AVVILVPLAAAP | 12 | 57.3 | 203.3 | 2.4 | Aliphatic |
| 109 | 465 | IAAVIVPVAALP | 12 | 50.2 | 203.3 | 2.4 | Aliphatic |
| 110 | 481 | AIAIAIVPVALP | 12 | 50.2 | 211.6 | 2.4 | Aliphatic |
| 111 | 482 | ILAVAAIPVAVP | 12 | 54.9 | 203.3 | 2.4 | Aliphatic |
| 112 | 483 | ILAAAIIPAALP | 12 | 54.9 | 204.1 | 2.2 | Aliphatic |
| 113 | 484 | LAVVLAAPAIVP | 12 | 50.2 | 203.3 | 2.4 | Aliphatic |
| 114 | 485 | AILAAIVPLAVP | 12 | 50.2 | 211.6 | 2.4 | Aliphatic |
| 115 | 501 | VIVALAVPALAP | 12 | 50.2 | 203.3 | 2.4 | Aliphatic |
| 116 | 502 | AAIIIVLPAALP | 12 | 50.2 | 203.3 | 2.4 | Aliphatic |
| 117 | 503 | LIVALAVPALAP | 12 | 50.2 | 220.0 | 2.4 | Aliphatic |
| 118 | 504 | LIVALAVPALAP | 12 | 50.2 | 211.7 | 2.4 | Aliphatic |
| 119 | 505 | AIIIVIAPAAAP | 12 | 50.2 | 195.8 | 2.3 | Aliphatic |
| 120 | 521 | LAALIVVPAVAP | 12 | 50.2 | 203.3 | 2.4 | Aliphatic |
| 121 | 522 | ALLVIAVPAVAP | 12 | 57.3 | 203.3 | 2.4 | Aliphatic |
| 122 | 524 | AVALIVVPALAP | 12 | 50.2 | 203.3 | 2.4 | Aliphatic |
| 123 | 525 | ALAIVVAPVAVP | 12 | 50.2 | 195.0 | 2.4 | Aliphatic |
| 124 | 541 | LLALIIAPAAAP | 12 | 57.3 | 204.1 | 2.1 | Aliphatic |
| 125 | 542 | ALALIIVPAVAP | 12 | 50.2 | 211.6 | 2.4 | Aliphatic |
| 126 | 543 | LLAALIAPAALP | 12 | 57.3 | 204.1 | 2.1 | Aliphatic |
| 127 | 544 | IVALIVAPAAVP | 12 | 43.1 | 203.3 | 2.4 | Aliphatic |
| 128 | 545 | VVLVLAAPAAVP | 12 | 57.3 | 195.0 | 2.3 | Aliphatic |
| 129 | 561 | AAVAIVLPAVVP | 12 | 50.2 | 195.0 | 2.4 | Aliphatic |
| 130 | 562 | ALIAAIVPALVP | 12 | 50.2 | 211.7 | 2.4 | Aliphatic |
| 131 | 563 | ALAVIVVPALAP | 12 | 50.2 | 203.3 | 2.4 | Aliphatic |
| 132 | 564 | VAIALIVPALAP | 12 | 50.2 | 211.7 | 2.4 | Aliphatic |
| 133 | 565 | VAIALIVPALAP | 12 | 50.2 | 195.0 | 2.4 | Aliphatic |
| 134 | 582 | VAVALIVPALAP | 12 | 50.2 | 203.3 | 2.4 | Aliphatic |
| 135 | 583 | AVILALAPIVAP | 12 | 50.2 | 211.6 | 2.4 | Aliphatic |
| 136 | 585 | ALIVAIAPALVP | 12 | 50.2 | 211.6 | 2.4 | Aliphatic |
| 137 | 601 | AAILIAVPIAAP | 12 | 57.3 | 195.8 | 2.3 | Aliphatic |
| 138 | 602 | VIVALAAPVLAP | 12 | 50.2 | 203.3 | 2.4 | Aliphatic |

TABLE 2-4
[Newly Developed Hydrophobic CPPs-240 aMTDs That All Critical Factors Are
Considered and Satisfied (Sequence ID No 139-184)]

| Sequence | | | | Rigidity/ | Sturctural | | |
|---|---|---|---|---|---|---|---|
| ID | | | | Flexibility | Feature | Hydropathy | Residue |
| Number | aMTD | Sequences | Length | (II) | (AI) | (GRAVY) | Structure |
| 139 | 603 | VLVALAAPVIAP | 12 | 57.3 | 203.3 | 2.4 | Aliphatic |
| 140 | 604 | VALIAVAPAVVP | 12 | 57.3 | 195.0 | 2.4 | Aliphatic |
| 141 | 605 | VIAAVLAPVAVP | 12 | 57.3 | 195.0 | 2.4 | Aliphatic |
| 142 | 622 | ALIVLAAPVAVP | 12 | 50.2 | 203.3 | 2.4 | Aliphatic |
| 143 | 623 | VAAAIALPAIVP | 12 | 50.2 | 187.5 | 2.3 | Aliphatic |
| 144 | 625 | ILAAAAAPLIVP | 12 | 50.2 | 195.8 | 202 | Aliphatic |
| 145 | 643 | LALVLAAPAIVP | 12 | 50.2 | 211.6 | 2.4 | Aliphatic |
| 146 | 645 | ALAVVALPAIVP | 12 | 50.2 | 203.3 | 2.4 | Aliphatic |
| 147 | 661 | AAILAPIVAALP | 12 | 50.2 | 195.8 | 2.2 | Aliphatic |
| 148 | 664 | ILIAIAIPAAAP | 12 | 54.9 | 204.1 | 2.3 | Aliphatic |
| 149 | 665 | LAIVLAAPVAVP | 12 | 50.2 | 203.3 | 2.3 | Aliphatic |
| 150 | 666 | AAIAIIAPAIVP | 12 | 50.2 | 195.8 | 2.3 | Aliphatic |
| 151 | 667 | LAVAIVAPALVP | 12 | 50.2 | 203.3 | 2.3 | Aliphatic |
| 152 | 683 | LAIVLAAPAVLP | 12 | 50.2 | 211.7 | 2.4 | Aliphatic |
| 153 | 684 | AAIVLALPAVLP | 12 | 50.2 | 211.7 | 2.4 | Aliphatic |
| 154 | 685 | ALLVAVLPAALP | 12 | 57.3 | 211.7 | 2.3 | Aliphatic |
| 155 | 686 | AALVAVLPVALP | 12 | 57.3 | 203.3 | 2.3 | Aliphatic |
| 156 | 687 | AILAVALPLLAP | 12 | 57.3 | 220.0 | 2.3 | Aliphatic |
| 157 | 703 | IVAVALVPALAP | 12 | 50.2 | 203.3 | 2.4 | Aliphatic |
| 158 | 705 | IVAVALLPALAP | 12 | 50.2 | 211.7 | 2.4 | Aliphatic |
| 159 | 706 | IVAVALLPAVAP | 12 | 50.2 | 203.3 | 2.4 | Aliphatic |
| 160 | 707 | IVALAVLPAVAP | 12 | 50.2 | 203.3 | 2.4 | Aliphatic |
| 161 | 724 | VAVLAVLPALAP | 12 | 57.3 | 203.3 | 2.3 | Aliphatic |
| 162 | 725 | IAVLAVAPAVLP | 12 | 57.3 | 203.2 | 2.3 | Aliphatic |
| 163 | 726 | LAVAIIAPAVAP | 12 | 57.3 | 187.5 | 2.2 | Aliphatic |
| 164 | 727 | VALAIALPAVLP | 12 | 57.3 | 211.6 | 2.3 | Aliphatic |
| 165 | 743 | AIAIALVPVALP | 12 | 57.3 | 211.6 | 2.4 | Aliphatic |
| 166 | 744 | AAVVIVAPVALP | 12 | 50.2 | 195.0 | 2.4 | Aliphatic |
| 167 | 746 | VAIIVVAPALAP | 12 | 50.2 | 203.3 | 2.4 | Aliphatic |
| 168 | 747 | VALLAIAPALAP | 12 | 57.3 | 195.8 | 2.2 | Aliphatic |
| 169 | 763 | VAVLIAVPALAP | 12 | 57.3 | 203.3 | 2.3 | Aliphatic |
| 170 | 764 | AVALAVLPAVVP | 12 | 57.3 | 195.0 | 2.3 | Aliphatic |
| 171 | 765 | AVALAVVPAVLP | 12 | 57.3 | 195.0 | 2.3 | Aliphatic |
| 172 | 766 | IVVIAVAPAVAP | 12 | 50.2 | 195.0 | 2.4 | Aliphatic |
| 173 | 767 | IVVAAVVPALAP | 12 | 50.2 | 195.0 | 2.4 | Aliphatic |
| 174 | 783 | IVALVPAVAIAP | 12 | 50.2 | 203.3 | 2.5 | Aliphatic |
| 175 | 784 | VAALPAVALVVP | 12 | 57.3 | 195.0 | 2.4 | Aliphatic |
| 176 | 786 | LVAIAPLAVLAP | 12 | 41.3 | 211.7 | 2.4 | Aliphatic |
| 177 | 787 | AVALVPVIVAAP | 12 | 50.2 | 195.0 | 2.4 | Aliphatic |
| 178 | 788 | AIAVAIAPVALP | 12 | 57.3 | 187.5 | 2.3 | Aliphatic |
| 179 | 803 | AIALAVPVLALP | 12 | 57.3 | 211.7 | 2.4 | Aliphatic |
| 180 | 805 | LVLIAAAPIALP | 12 | 41.3 | 220.0 | 2.4 | Aliphatic |
| 181 | 806 | LVALAVPAAVLP | 12 | 57.3 | 203.3 | 2.3 | Aliphatic |
| 182 | 807 | AVALAVPALVLP | 12 | 57.3 | 203.3 | 2.3 | Aliphatic |
| 183 | 808 | LVVLAAAPLAVP | 12 | 41.3 | 203.3 | 2.3 | Aliphatic |
| 184 | 809 | LIVLAAPALAAP | 12 | 50.2 | 195.8 | 2.2 | Aliphatic |

TABLE 2-5
[Newly Developed Hydrophobic CPPs-240 aMTDs That All Critical Factors Are
Considered and Satisfied (Sequence ID No 185-230)]

| Sequence | | | | Rigidity/ | Sturctural | | |
|---|---|---|---|---|---|---|---|
| ID | | | | Flexibility | Feature | Hydropathy | Residue |
| Number | aMTD | Sequences | Lenght | (II) | (AI) | (GRAVY) | Structure |
| 185 | 810 | VIVLAAPALAAP | 12 | 50.2 | 187.5 | 2.2 | Aliphatic |
| 186 | 811 | AVVLAVPALAVP | 12 | 57.3 | 195.0 | 2.3 | Aliphatic |
| 187 | 824 | LIIVAAAPAVAP | 12 | 50.2 | 187.5 | 2.3 | Aliphatic |
| 188 | 825 | IVAVIVAPAVAP | 12 | 43.2 | 195.0 | 2.5 | Aliphatic |
| 189 | 826 | LVALAAPIIAVP | 12 | 41.3 | 211.7 | 2.4 | Aliphatic |
| 190 | 827 | IAAVLAAPALVP | 12 | 57.3 | 187.5 | 2.2 | Aliphatic |
| 191 | 828 | IALLAAPIIAVP | 12 | 41.3 | 220.0 | 2.4 | Aliphatic |
| 192 | 829 | AALALVAPVIVP | 12 | 50.2 | 203.3 | 2.4 | Aliphatic |
| 193 | 830 | IALVAAPVALVP | 12 | 57.3 | 203.3 | 2.4 | Aliphatic |
| 194 | 831 | IIVAVAPAAIVP | 12 | 43.2 | 203.3 | 2.5 | Aliphatic |
| 195 | 832 | AVAAIVPVIVAP | 12 | 43.2 | 195.0 | 2.5 | Aliphatic |
| 196 | 843 | AVLVLVAPAAAP | 12 | 41.3 | 219.2 | 2.5 | Aliphatic |
| 197 | 844 | VVALLAPLIAAP | 12 | 41.3 | 211.8 | 2.4 | Aliphatic |
| 198 | 845 | AAVVIAPLLAVP | 12 | 41.3 | 203.3 | 2.4 | Aliphatic |
| 199 | 846 | IAVAVAAPLLVP | 12 | 41.3 | 203.3 | 2.4 | Aliphatic |
| 200 | 847 | LVAIVVLPAVAP | 12 | 50.2 | 219.2 | 2.6 | Aliphatic |
| 201 | 848 | AVAIVVLPAVAP | 12 | 50.2 | 195.0 | 2.4 | Aliphatic |
| 202 | 849 | AVILLAPLIAAP | 12 | 57.3 | 220.0 | 2.4 | Aliphatic |
| 203 | 850 | LVIALAAPVALP | 12 | 57.3 | 211.7 | 2.4 | Aliphatic |
| 204 | 851 | VLAVVLPAVALP | 12 | 57.3 | 219.2 | 2.5 | Aliphatic |
| 205 | 852 | VLAVAAPAVLLP | 12 | 57.3 | 203.3 | 2.3 | Aliphatic |
| 206 | 863 | AAVVLLPIIAAP | 12 | 41.3 | 211.7 | 2.4 | Aliphatic |
| 207 | 864 | ALLVIAPAIAVP | 12 | 57.3 | 211.7 | 2.4 | Aliphatic |
| 208 | 865 | AVLVIAVPAIAP | 12 | 57.3 | 203.3 | 2.5 | Aliphatic |
| 209 | 867 | ALLVVIAPLAAP | 12 | 41.3 | 211.7 | 2.4 | Aliphatic |
| 210 | 868 | VLVAAILPAAIP | 12 | 54.9 | 211.7 | 2.4 | Aliphatic |
| 211 | 870 | VLVAAVLPIAAP | 12 | 41.3 | 203.3 | 2.4 | Aliphatic |
| 212 | 872 | VLAAAVLPLVVP | 12 | 41.3 | 219.2 | 2.5 | Aliphatic |
| 213 | 875 | AIAIVVPAVAVP | 12 | 50.2 | 195.0 | 2.4 | Aliphatic |
| 214 | 877 | VAIIAVPAVVAP | 12 | 57.3 | 195.0 | 2.4 | Aliphatic |
| 215 | 878 | IVALVAPAAVVP | 12 | 50.2 | 195.0 | 2.4 | Aliphatic |
| 216 | 879 | AAIVLLPAVVVP | 12 | 50.2 | 219.1 | 2.5 | Aliphatic |
| 217 | 881 | AALIVVPAVAVP | 12 | 50.2 | 195.0 | 2.4 | Aliphatic |
| 218 | 882 | AIALVVPAVAVP | 12 | 57.3 | 195.0 | 2.4 | Aliphatic |
| 219 | 883 | LAIVPAAIAALP | 12 | 50.2 | 195.8 | 2.2 | Aliphatic |
| 220 | 885 | LVAIAPAVAVLP | 12 | 57.3 | 203.3 | 2.4 | Aliphatic |
| 221 | 887 | VLAVAPAVAVLP | 12 | 57.3 | 195.0 | 2.4 | Aliphatic |
| 222 | 888 | ILAVVIAPAAAP | 12 | 54.9 | 187.5 | 2.3 | Aliphatic |
| 223 | 889 | ILVAAAPIAALP | 12 | 57.3 | 195.8 | 2.2 | Aliphatic |
| 224 | 891 | ILAVAAIPAALP | 12 | 54.9 | 195.8 | 2.2 | Aliphatic |
| 225 | 893 | VIAIPAILAAAP | 12 | 54.9 | 195.8 | 2.3 | Aliphatic |
| 226 | 895 | AIIIVVPAIAAP | 12 | 50.2 | 211.7 | 2.5 | Aliphatic |
| 227 | 896 | AILIVVAPIAAP | 12 | 50.2 | 211.7 | 2.5 | Aliphatic |
| 228 | 897 | AVIVPVAIIAAP | 12 | 50.2 | 203.3 | 2.5 | Aliphatic |
| 229 | 899 | AVVIALPAVVAP | 12 | 57.3 | 195.0 | 2.4 | Aliphatic |
| 230 | 900 | ALVAVIAPVVAP | 12 | 57.3 | 195.0 | 2.4 | Aliphatic |

TABLE 2-6
[Newly Developed Hydrophobic CPPs-240 aMTDs That All Critical Factors Are
Considered and Satisfied (Sequence ID No 231-240)]

| Sequence | | | | Rigidity/ | Sturctural | | |
|---|---|---|---|---|---|---|---|
| ID | | | | Flexibility | Feature | Hydropathy | Residue |
| Number | aMTD | Sequences | Lenght | (II) | (AI) | (GRAVY) | Structure |
| 231 | 901 | ALVAVLPAVAVP | 12 | 57.3 | 195.0 | 2.4 | Aliphatic |
| 232 | 902 | ALVAPLLAVAVP | 12 | 41.3 | 203.3 | 2.3 | Aliphatic |
| 233 | 904 | AVLAVVAPVVAP | 12 | 57.3 | 186.7 | 2.4 | Aliphatic |
| 234 | 905 | AVIAVAPLVVAP | 12 | 41.3 | 195.0 | 2.4 | Aliphatic |
| 235 | 906 | AVIALAPVVVAP | 12 | 57.3 | 195.0 | 2.4 | Aliphatic |
| 236 | 907 | VAIALAPVVVAP | 12 | 57.3 | 195.0 | 2.4 | Aliphatic |
| 237 | 908 | VALALAPVVVAP | 12 | 57.3 | 195.0 | 2.3 | Aliphatic |
| 238 | 910 | VAALLPAVVVAP | 12 | 57.3 | 195.0 | 2.3 | Aliphatic |
| 239 | 911 | VALALPAVVVAP | 12 | 57.3 | 195.0 | 2.3 | Aliphatic |
| 240 | 912 | VALLAPAVVVAP | 12 | 57.3 | 195.0 | 2.3 | Aliphatic |
| | | | | 52.6 ± 5.1 | 201.7 ± 7.8 | 2.3 ± 0.1 | |

TABLE 3
[Summarized Critical Factors of aMTD After In-Depth
Analysis of Experimental Results]
Summarized Critical Factors of aMTD

| | Analysis of Experimental Results |
|---|---|
| Critical Factor | Range |
| Bending Potential | Proline presences in the middle (5′, 6′, 7′ or 8′) |
| (Proline Position: PP) | and at the end (12′) of peptides |
| Rigidity/Flexibility | 41.3-57.3 |
| (Instability Index: II) | |
| Structural Feature | 187.5-220.0 |
| (Aliphatic Index: AI) | |
| Hydropathy | 2.2-2.6 |
| (Grand Average of | |
| Hydropathy GRAVY) | |
| Length | 12 |
| (Number of Amino Acid) | |
| Amino acid Composition | A, V, I, L, P |

TABLE 4
[Amino Acid and Nucleotide Sequence of Newly Developed Advanced MTDs
Which Follow All Critical Factors]

| ID | Amino Acid Sequence | NucleotideSequence |
|---|---|---|
| 61 | VAALPVLLAALP | GTG GCG GCG CTG CCG GTG CTG CTG GCG GCG CTG CCG |
| 65 | ALAVPVALAIVP | GCG CTG GCG GTG CCG GTG GCG CTG GCG ATT GTG CCG |

These examined critical factors are within the range that we have set for our critical factors; therefore, we were able to confirm that the aMTDs that satisfy these critical factors have much higher cell-permeability and intracellular delivery potential compared to reference hydrophobic CPPs reported during the past two decades.

2. Development of GPX7 Recombinant Proteins Fused to aMTD and Solubilization Domain

2-1. Design of Novel Hydrophobic CPPs-aMTDs for Development of GPX7 Recombinant Proteins

Based on these six critical factors proven by experimental data, newly designed advanced macromolecule transduction domains (aMTDs) have been developed, and optimized for their practical therapeutic usage to facilitate protein translocation across the membrane. For this present invention, cell-permeable GPX7 recombinant proteins have been developed by adopting aMTD61 and aMTD165 (TABLE 5) that satisfied all 6 critical factors.

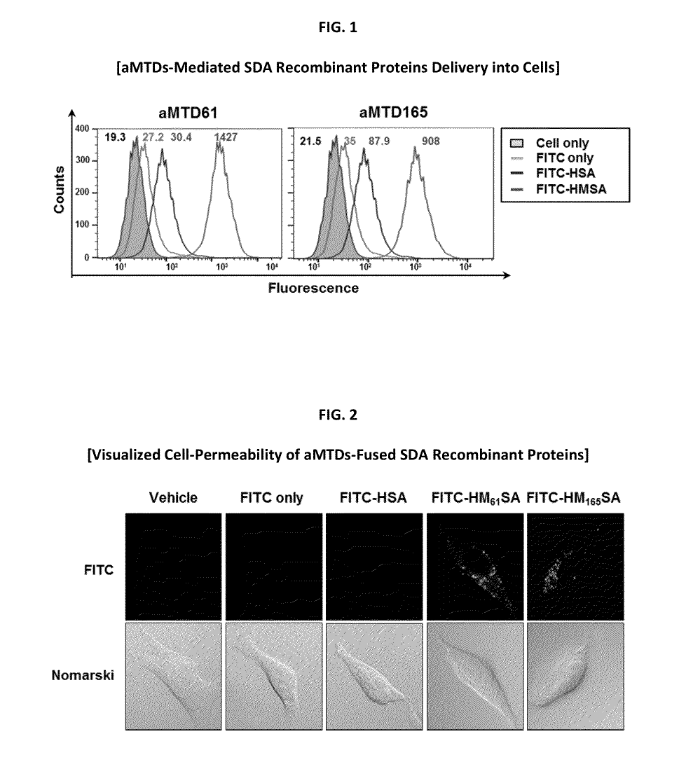

To determine the protein transduction activity of these aMTDs, histidine-tagged solubilization domain A (SDA)—a non-functional and soluble protein present in a spore surface coat of Myxococcus xanthus having tandem repeats of 2 N-terminal domain (NTD) sequences of CP_{—}000113.1-recombinant proteins including aMTDs were designed. SDA recombinant proteins were conjugated to 5/6-fluorescein isothiocyanate (FITC) and cellular uptake of FITC-labeled SDA recombinant proteins were measured by flow cytometry (FIG. 1) and confocal microscopy (FIG. 2). Both of aMTD61 and aMTD165 significantly promoted the uptake of SDA cargo protein into cultured cells compared with SDA recombinant proteins lacking aMTD (HSA; FIGS. 1 and 2) and their relative cell permeability to the HSA recombinant proteins was 46.9 and 10.3 fold, respectively (FIG. 1). Therefore, we selected these 2 aMTDs (aMTD61 and aMTD165) having cell-permeability for development of CP-GPX7 recombinant protein.

TABLE 5
[Critical Factors of aMTD61 and 165]

| | | | | Bending Potential | | | Rigidity/ | Sturctural | |
|---|---|---|---|---|---|---|---|---|---|
| | | | | Prolin Position | | | Flexibility | Feature | Hydropathy |
| No. | Length | Theoretical pl | M.W. | 5′ | 6′ | 12′ | (II) | (Al) | (GRAVY) |
| 61 | 12 | 5.49 | 1147.4 | 1 | 0 | 1 | 57.3 | 211.7 | 2.3 |
| 165 | 12 | 5.57 | 1133.4 | 1 | 0 | 1 | 50.2 | 203.3 | 2.4 |

2-2. Selection of Solubilization Domain for Recombinant Proteins

Since recombinant cargo proteins fused to previously developed hydrophobic CPP—MTS, MTM and MTDs—were highly insoluble and had extremely low solubility, we additionally adopted non-functional solubilization domains (SDs) to improve solubility, yield and stability of the recombinant proteins and hypothesized that fusion of GPX7 with SDs and novel hydrophobic CPP, aMTD, would greatly increase solubility/yield and cell-/tissue-permeability of recombinant cargo proteins for the clinical application. According to this specific aim, 5 solubilization domains were selected and information of these SDs are shown TABLE 6.

TABLE 6
[Informations of Solubilization Domains]

| | | | Protein | | Instability | |
|---|---|---|---|---|---|---|
| SD | Genbank ID | Origin | (kDa) | pl | Index (II) | GRAVY |
| A | CP000113.1 | Bacteria | 23 | 4.6 | 48.1 | −0.1 |
| B | BC086945.1 | Pansy | 11 | 4.9 | 43.2 | −0.9 |
| C | CP012127.1 | Human | 12 | 5.8 | 30.7 | −0.1 |
| D | CP012127.1 | Bacteria | 23 | 5.9 | 26.3 | −0.1 |
| E | CP011550.1 | Human | 11 | 5.3 | 44.4 | −0.9 |
| F | NG_034970 | Human | 34 | 7.1 | 56.1 | −0.2 |

2-3. Preparation of Recombinant aMTD-GPX7-SD Fusion Proteins

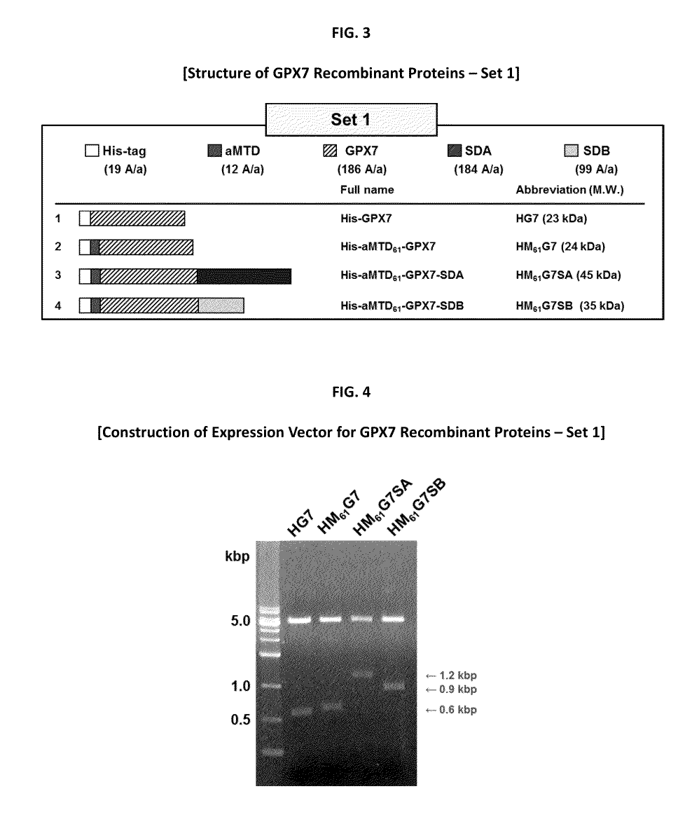

We first designed 4 different kinds of recombinant proteins either with or without the aMTD61 and SDs for GPX7 recombinant proteins (set 1). Each recombinant protein of set 1 was named His-GPX7 (HG7), His-aMTD61-GPX7 (HM61G7), His-aMTD61-GPX7-SDA (HM61G7SA) and His-aMTD61-GPX7-SDB (HM61G7SB; FIG. 3, 4). The designed GPX7 recombinant proteins (HG7, HM61G7, HM61G7SA and HM61G7SB) were expressed in bacterial system and purified. All of GPX7 recombinant proteins (set 1) were appeared the precipitation. The solubility of each soluble GPX7 recombinant proteins was determined by measuring absorbance (A450) and scored on a 1 point scale (FIG. 5). In respect of solubility and yield, set 1 for development of CP-GPX7 were not improved at all. PCR primers using set 1 cloning are lined up in TABLES 7, 8 and 9.

TABLE 7
[PCR Primers for His-Tagged GPX7 Proteins]

| Cargo | aMTD ID | Recombinant Protein | 5′ Primers | 3′ Primers |
|---|---|---|---|---|
| GPX7 | — | HG7 | 5′-GGAATTCCATATGGTGGCGGCGACGG | 5′-CCCGGATCCTTATAAGTCTTCTCGCTTC |
| | | | TGGCAGCGGCGTGG-3′ | AGTAGGATGAG-3′ |
| | 61 | HM₈₁G7 | 5′-GGAATTCCATATGGTGGCGGCGCTGC | 5′-CCCGGATCCTAAGTCTTCTCGCTTCAGT |
| | 61 | HM₆₁G7SA | CGGTGCTGCTGGCGGCGCTGCCGGTGGCG | AGGATGAG CTT-3′ |
| | 61 | HM₆₁G7SB | GCGACGGTGGCAGCGGCGTGG-3′ | |
| | 165 | H5AM₁₆₅G7SB | 5′-CCGCATATGGCGCTGGTGCCGGTGGC | 5′-ACGCGTCGACTAAGTCTTCTCGCTTCAG |
| | | | GCTGGCGATTGTGCCGGTGGCGGCGACGG | TAGGATGAG-3′ |
| | | | TGGCAGCGGCGTGG-3′ | |
| | — | HSAG7SB | 5′-CCGCATATGGTGGCGGCGACGGTGGC | |
| | | | AGCGGCGTGG-3′ | |
| | 165 | SCHG7M₁₆₃ | 5′-CGGGATCCGTGGCGGCGACGGTGGCA | 5′-CCGCTCGAGTTACGGCACAATCGCCAGC |
| | | | GCGGCGTGG-3′ | GCCACCGGCACCGCCAGCGCTAAGTCTTCTC |
| | 165 | SDHG7M₁₆₅ | 5′-CGCGAACAGATTGGAGGTGTGGCGGC | GCTTCAGTAGGATGAG-3′ |
| | | | GACGGTGGCAGCGGCGTGG-3′ | |
| | 165 | M₁₆₅G7SFH | 5′-GGAATTCATGGCGCTGGCGGTGCCGG | 5′-CCGCTCGAGTAAGTCTTCTCGCTTCAGT |
| | | | TGGCGCTGGCGATTGTGCCGGTGGCGGCG | AGGATGAG-3′ |
| | | | ACGGTGGCAGCGGCGTGG-3′ | |

TABLE 8
[PCR Primers for aMTD/SDA-Fused GPX7 Proteins]

| Cargo | SD | Recombinant Protein | 5′ Primers | 3′ Primers |
|---|---|---|---|---|
| GPX7 | SDA | HM₆₁G7SA | 5′-CCCGGATCCATGGCAAATATTACCGTTT | 5′-CGCGTCGACTTACCTCGGCTGCACCGGC |
| | | | TCTATAACGAA-3′ | ACGGAGATGAC-3′ |
| | | HSAM₁₆₅G7SB | 5′-GGGTTTCATATGATGGCAAATATTACCG | 5′-CGCGGATCCCCTCGGCTGCACCGGCACG |
| | | | TTTTC-3′ | G-3′ |
| | | HSAG7SB | | |
| | | HSAM₁₆₅SB | | 5′-ACGCGTCGACCGGCACAATCGCCAGCGC |
| | | | | CACCGGCACCGCCAGCGCCCTCGGCTGCACC |
| | | | | GGCACGGA-3′ |

TABLE 9
[PCR Primers for aMTD/SDB-Fused GPX7 Proteins]

| Cargo | SD | Recombinant Protein | 5′ Primers | 3′ Primers |
|---|---|---|---|---|
| GPX7 | SDB | HM₆₁G7SB | 5′-CCCGGATCCATGGCAGAACAAAGCGA | 5′-CGCGTCGACTTAAAGGGTTTCCGAAG |
| | | | CAAGGATGTGAAG-3′ | GCTTGGCTATCTT-3′ |
| | | HSAM₁₆₈G7SB | 5′-ACGCGTCGACATGGCAGAACAAAGCG | 5′-CCGCTCGAGGTTAAAGGGTTTCCGAA |
| | | HSAG7SB | AC-3′ | GGCTTG-3′ |
| | | HSAM₁₆₅SB | | |

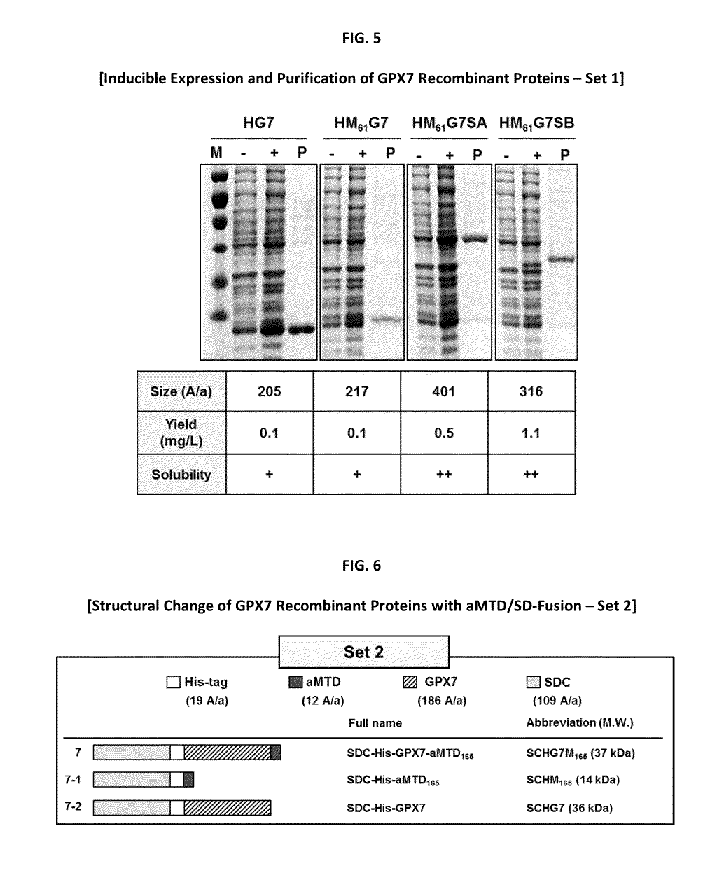

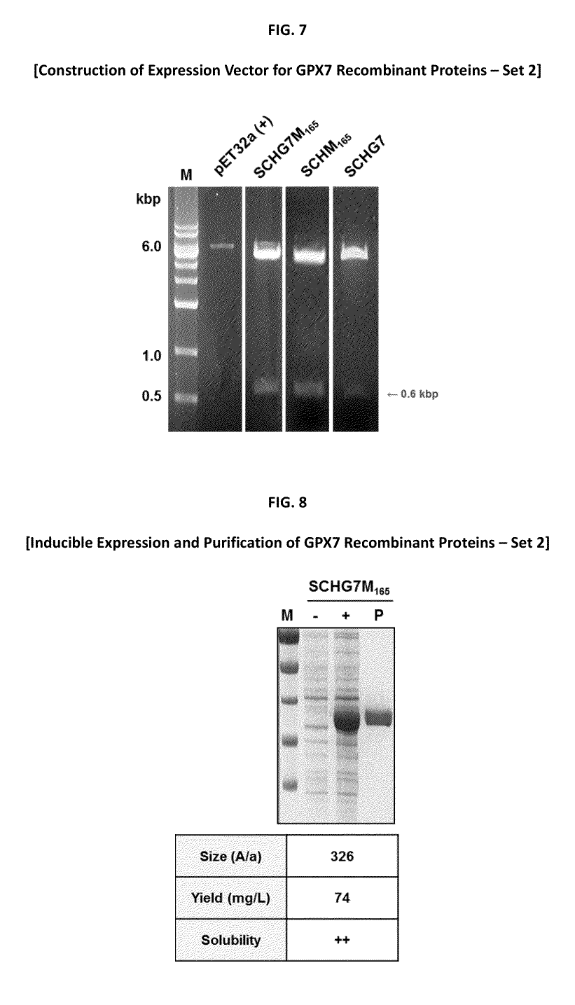

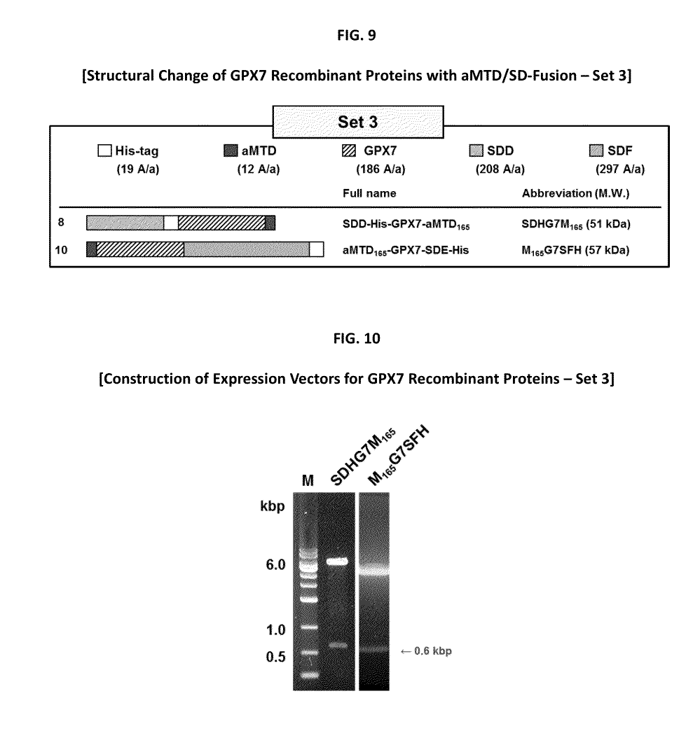

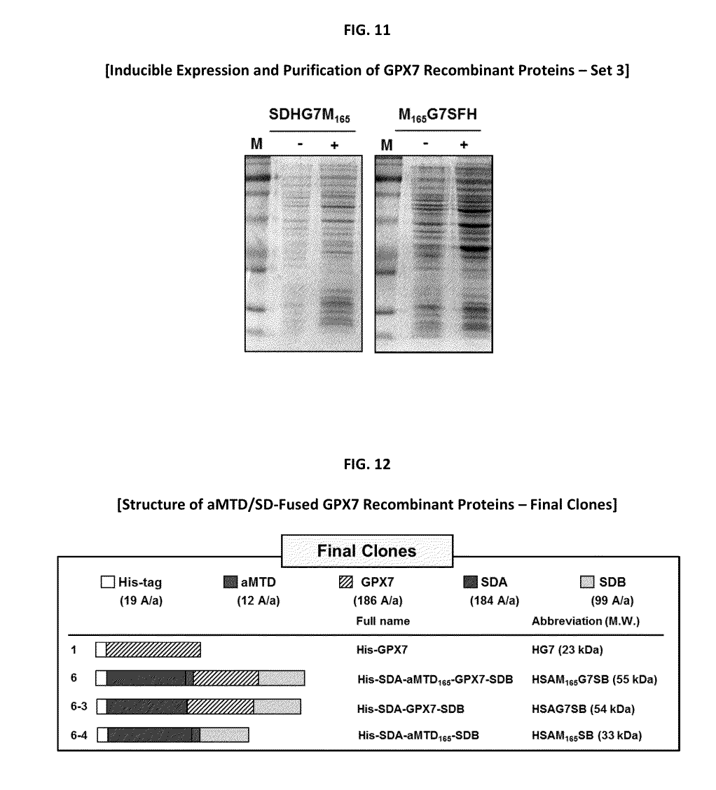

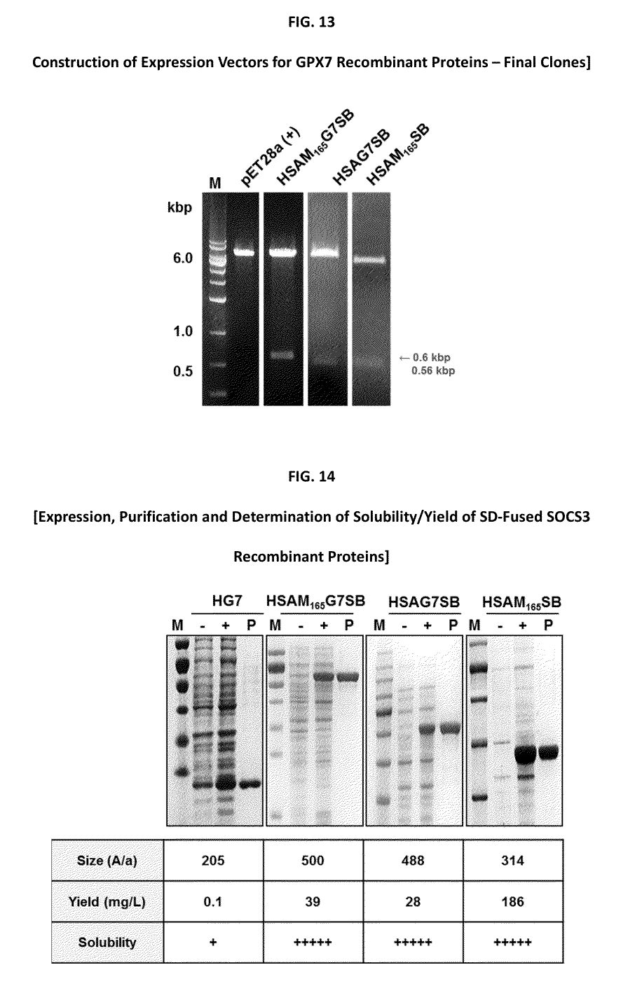

To develop stable CP-GPX7, aMTD61 and solubilization domains were replaced to aMTD165 and SDC fused to N terminus, respectively. Also, we designed 2 negative controls—recombinant proteins lacking GPX7 cargo protein (SDC-His-aMTD165; SCHM165) and aMTD (SDC-His-GPX7; SCHG7). In addition, solubilization domain was changed to SDD and SDF (FIGS. 6, 7, 9 and 10). All GPX7 recombinant proteins could still not solve the problems of solubility and yield. Nucleotide and amino acid sequences of SDs are presented in SEQ ID NOs: 489, 490, 491, 492, 493, 494, 495, 496, 497, 498, 499 and 500, separately. PCR primers using set 2 and set 3 cloning are lined up in TABLE 7.

We subsequently generated GPX7 recombinant protein which containing both of SDA and SDB (HSAM₁₆₅G7SB). In addition, GPX7 protein lacking aMTD (HSAG7SB) and GPX7 recombinant protein lacking GPX7 (HSAM₁₆₅SB) were designed (FIG. 12). The GPX7 recombinant proteins were expressed, purified, and prepared in soluble form (FIG. 14). GPX7 recombinant proteins containing aMTD165 and solubilization domains (HSAM₁₆₅G7SB) had little tendency to precipitate and solubility of aMTD/SDs-fused GPX7 proteins was scored on a 5 point scale (FIG. 14).

Yields per L of E. coli for each GPX7 recombinant protein (mg/L) ranged from 0.1 to 39 mg/L (FIG. 4). Yields of GPX7 proteins containing an aMTD and SDs (HSAM₁₆₅G7SB) were 390% higher than his-tagged GPX7 protein (HG7).

3. aMTD/SDs-Fused GPX7 Recombinant Proteins Significantly Increase Cell- and Tissue-Permeability

3-1. aMTD/SDs-Fused GPX7 Recombinant Proteins are Cell-Permeable

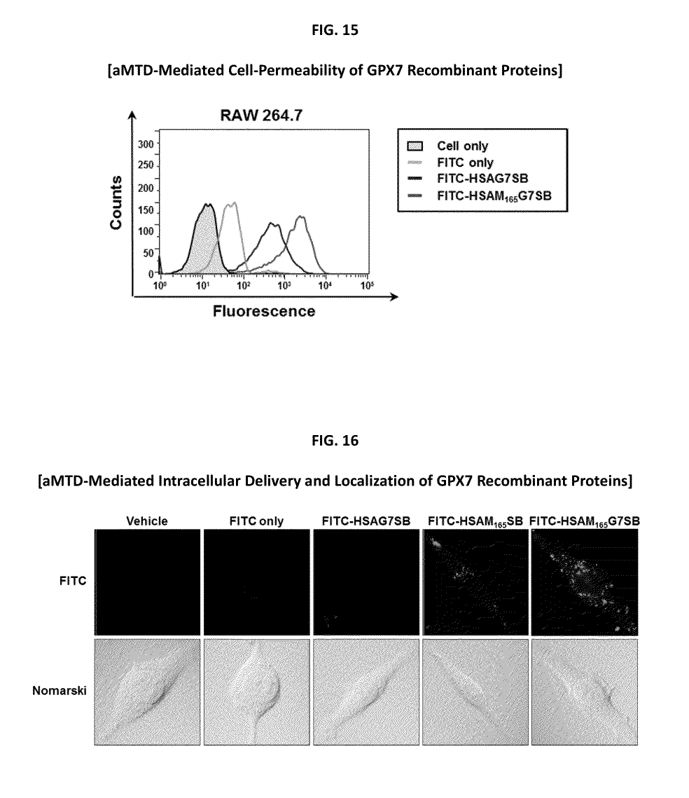

To examine the protein uptake, the GPX7 recombinant proteins were conjugated to FITC and cells were treated with 10 μM FITC-labeled GPX7 recombinant proteins. Internalized proteins were measured by flow cytometry (FIG. 15) and visualized by confocal laser scanning microscopy (FIG. 16). GPX7 proteins containing aMTD165 (HSAM₁₆₅G7SB) efficiently entered the cells (FIGS. 15 and 16) and were localized to various extents in cytoplasm (FIG. 16). In contrast, GPX7 protein (HSAG7SB) lacking aMTD did not appear to enter cells.

3-2. aMTD/SDs-Fused GPX7 Recombinant Proteins Enhance the Systemic Delivery to a Variety of Tissues

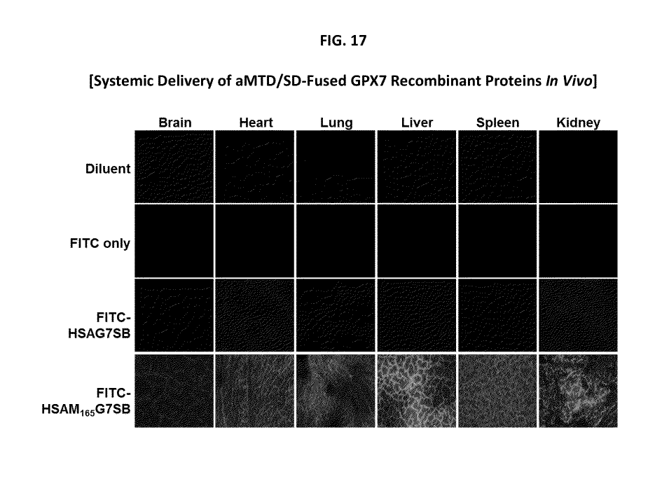

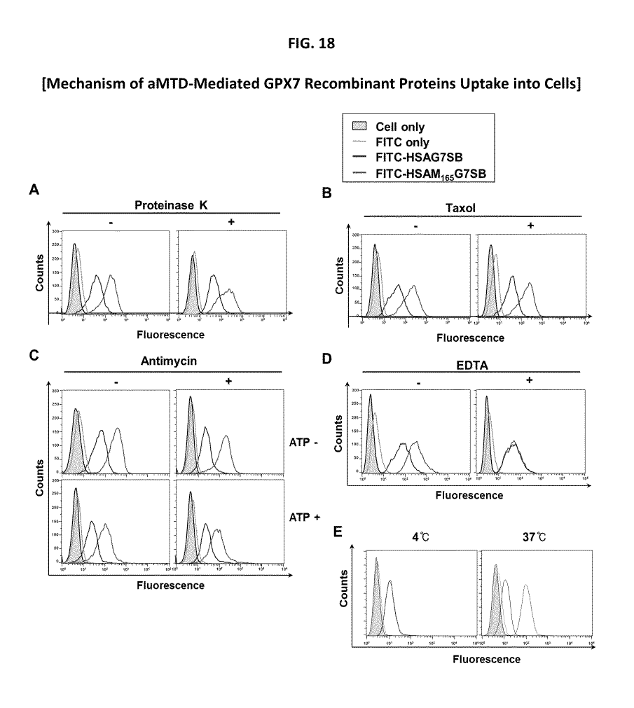

To further investigate in vivo delivery of GPX7 recombinant proteins, FITC-labeled GPX7 proteins were monitored following intraperitoneal (IP) injections in mice. Tissue distributions of fluorescence-labeled-GPX7 proteins in different organs was analyzed by fluorescence microscopy (FIG. 17). GPX7 recombinant proteins fused to aMTD165 (HSAM₁₆₅G7SB) were distributed to a variety of tissues (liver, kidney, spleen, lung, heart and, to a lesser extent, brain). Predictably, liver showed highest levels of fluorescent cell-permeable GPX7 since intraperitoneal administration favors the delivery of proteins to this organ via the portal circulation. GPX7 containing aMTD165 was detectable to a lesser degree in lung, spleen and heart. These data suggest that GPX7 protein containing both of aMTD165 and SDs leads to high cell-/tissue-permeability due to the increase in solubility and stability of the protein, and it displayed a dramatic synergic effect on cell-/tissue-permeability.

3-3. Cell Permeability Mediated aMTD165 was Achieved Efficiently Depend on Stable Plasma Bi-Lipid Layer Membrane

We next investigated how of aMTD165-mediated intracellular delivery was occurred. The aMTD-mediated intracellular delivery of GPX7 protein did not require protease-sensitive protein domains displayed on the cell surface (FIG. 10A), microtubule function (FIG. 10B), or ATP utilization (FIG. 10C), since aMTD165-dependent uptake [compare to HSAG7SB (blue)] was essentially unaffected by treating cells with proteinase K, taxol, or the ATP depleting agent, antimycin. Conversely, aMTD165-fused GPX7 proteins uptake was blocked by treatment with EDTA and low temperature (FIGS. 10D and E), indicating the importance of membrane integrity and fluidity for aMTD-mediated protein transduction.

4. aMTD/SD-Fused GPX7 Protein Efficiently Inhibits Cellular Processes

4-1. CP-GPX7 Inhibited TNF-α Induced NF-κB Nuclear Translocation

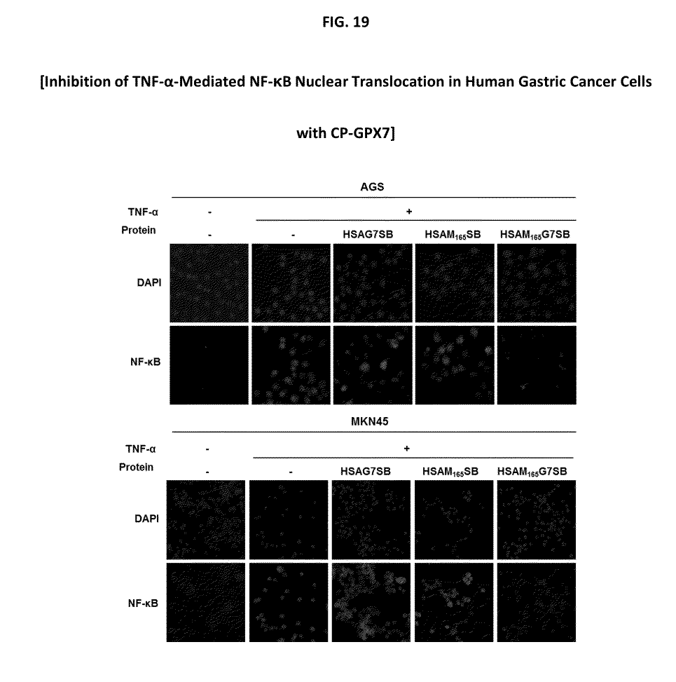

The ultimate test of cell-penetrating efficiency is a determination of intracellular activity of GPX7 proteins transported by aMTD. Since GPX7 are known to inhibit the TNF-α-induced NF-κB activation in GIT-specific cancer, we demonstrated whether cell-permeable GPX7 inhibits the translocation of NF-κB into the nucleus. As shown in FIG. 19, HSAM₁₆₅G7SB efficiently inhibited the nuclear localization of NF-κB after treatment with TNFα in human gastric cancer cells (AGS and MKN45), indicating the suppression of TNF-α mediated-NF-κB activation. In contrast, GPX7 recombinant proteins lacking aMTD165 or GPX7 (HSAG7SB and HSAM₁₆₅SB) did not affect the translocation of NF-κB into nuclear, indicating that HSAG7SB, which lacks an aMTD sequence and did not enter the cells, has no biological activity.

4-2. CP-GPX7 Suppressed the Phosphorylation of NF-κB in Gastric Cancer Cells

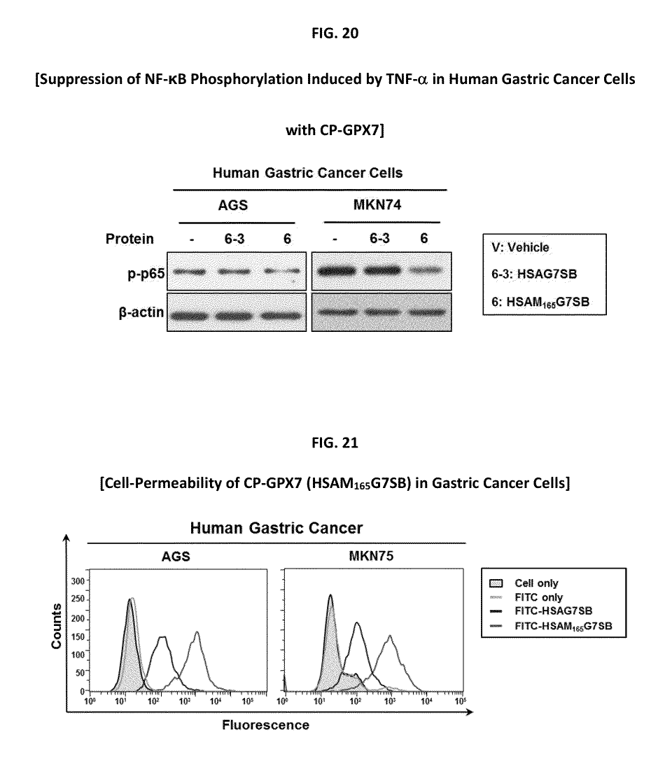

We next investigated the effect of cell-permeable GPX7 proteins on phosphorylation of NF-κB. Treatment of human gastric cancer cells (AGS and MKN74) with GPX7 proteins containing aMTD165 and SDs (HSAM₁₆₅G7SB) significantly suppressed the on phosphorylation of NF-κB (FIG. 20), indicating the inhibition of NF-κB activation. In contrast, NF-κB activity in human gastric cancer cells treated with non-permeable GPX7 protein (HSAG7SB) was unchanged, indicating that recombinant GPX7 lacking the aMTD doesn't affect intracellular signaling. Therefore, we conclude that differences in the biological activities of HSAM₁₆₅G7SB as compared to HSAG7SB are due to the differences in protein uptake mediated by the aMTD sequence. In light of solubility/yield, cell-/tissue-permeability and biological effect, GPX7 recombinant protein containing aMTD and SDs (HSAM₁₆₅G7SB) is a prototype of a new generation of cell-permeable GPX7 (CP-GPX7), and will be selected for further evaluation as a potential anti-tumor agent in GIT cancer.

### 5. CP-GPX7 Suppresses Pro-Tumorigenic Functions GIT Cells

5-1. CP-GPX7 Enhances the Penetration into Gastric Cancer Cells and Systemic Delivery to Stomach

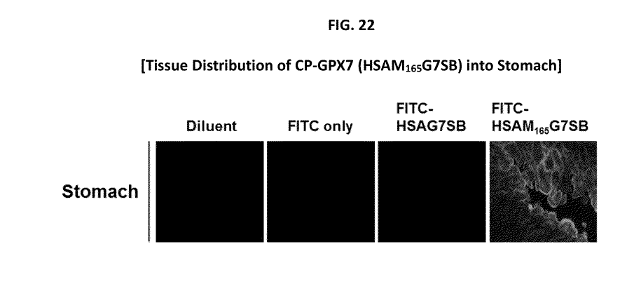

To determine the cell-permeability of CP-GPX7 in the gastric cancer cells, cellular uptake of FITC-labeled GPX7 recombinant proteins was quantitatively evaluated by flow cytometry. FITC-HSAM₁₆₅G7SB recombinant protein (CP-GPX7) promoted the transduction into cultured AGS and MKN75 gastric cancer cells (FIG. 22). In addition, CP-GPX7 enhanced the systemic delivery to stomach after intraperitoneal injection (FIG. 21). Therefore, these data indicate that CP-GPX7 could be intracellularly delivered and distributed to the target cells and stomach tissue, contributing for beneficial biotherapeutic effects.

5-2. CP-GPX7 Inhibits Cell Viability in GIT-Cancer Cells

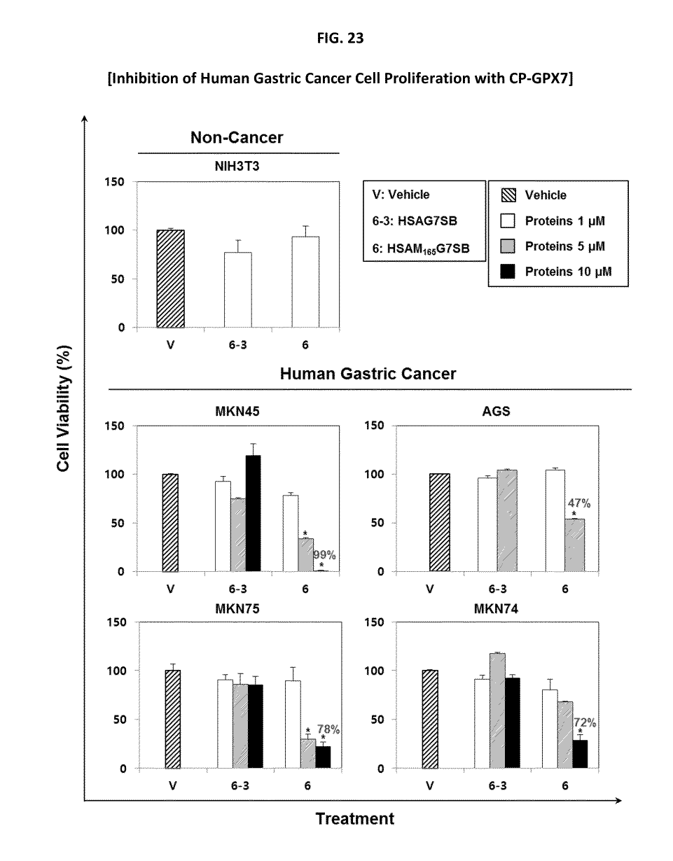

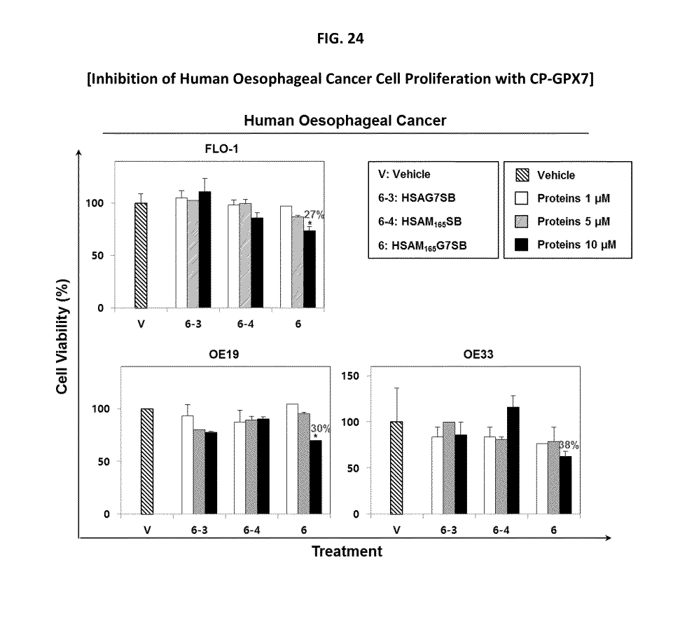

Since the endogenous level of GPX7 protein is reduced in GIT-cancer cells and reconstitution of GPX7 significantly suppresses growth and proliferation of the cells, we therefore examined the effects of CP-GPX7 on survival in human gastric and oesophageal cancer cell lines. (FIGS. 23 and 24). Human gastric and oesophageal cancer cells were incubated in normal culture media and treated with the GPX7 recombinant proteins fused to aMTD and SDs (HSAM₁₆₅G7SB; CP-GPX7) or a GPX7 recombinant lacking an aMTD (HSAG7SB). We also compared the ability of CP-GPX7 to induce cytotoxicity in non-cancer cells originated from mouse fibroblast (NIH3T3). As shown in FIGS. 23 and 24, GPX7 recombinant proteins containing aMTD165 significantly suppressed cell proliferation in both of gastric and oesophageal cancer cells. HSAM₁₆₅G7SB (CP-GPX7) protein was the most cytotoxic to MKN45 gastric cancer cells—over 99% in 10 μM treatment (p<0.01)—especially compared to vehicle alone (i.e. exposure of cells to culture media without recombinant proteins; FIG. 23). In addition, MKN74 and MKN75 cells also showed over 70% suppression on cell viability in treatment with CP-GPX7. However, neither cell-permeable GPX7 protein adversely affected the cell viability of non-cancer cells (NIH3T3) even after exposing these cells over 4 days (FIG. 23). These results suggest that the CP-GPX7 protein is not overly toxic to normal cells and selectively kills tumor cells, and would have a great ability to inhibit cell survival-associated phenotypes in GIT-cancer cells without any severe aberrant effects as a protein-based biotherapeutics.

5-3. CP-GPX7 Regulates the Expression of Cell Cycle-Associated Proteins in Gastric Cancer Cells.

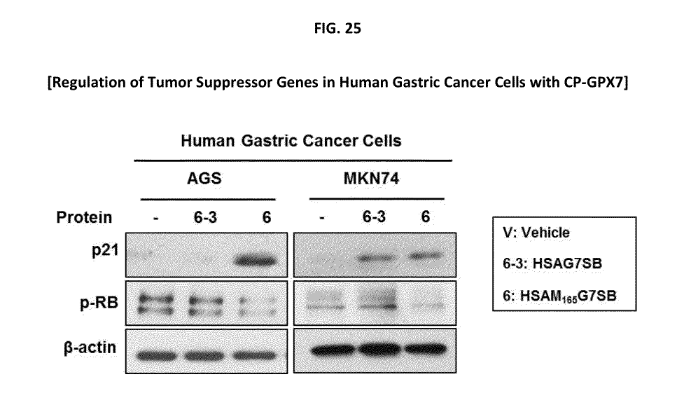

The CP-GPX7 appeared to be biological active, as HSAM₁₆₅G7SB-treated cells expressed lower levels of phosphorylated retinoblastoma tumor suppressor (RB) and higher levels of p21, as compared to HSAG7SB-treated cells (FIG. 25). These results suggest that CP-GPX7 inhibits cell cycle in cancer cells, leading to suppress the GIT-cancer cell proliferation.

5-4. CP-GPX7 Induces Apoptosis in GIT-Cancer Cells

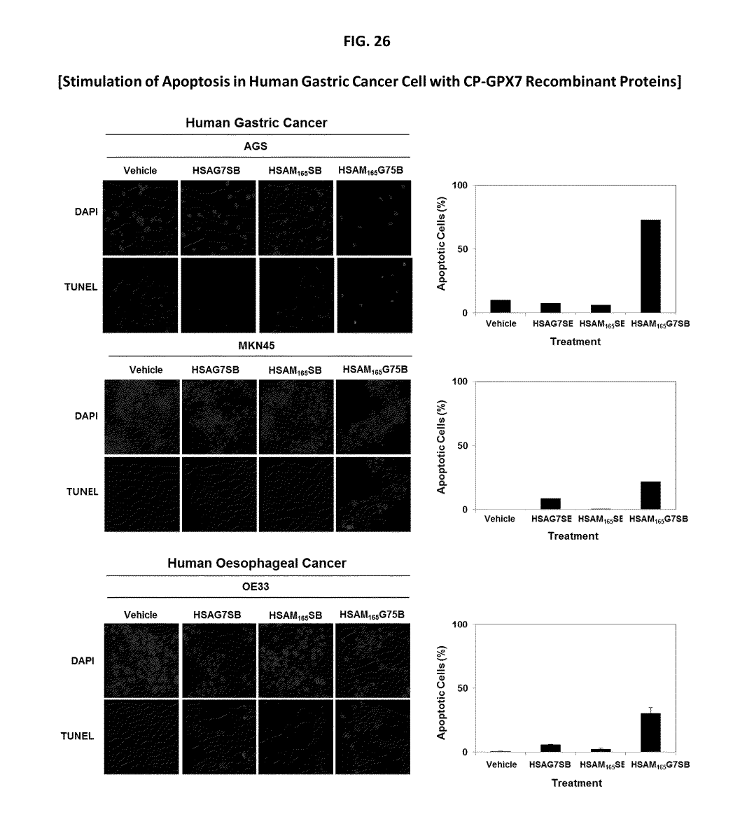

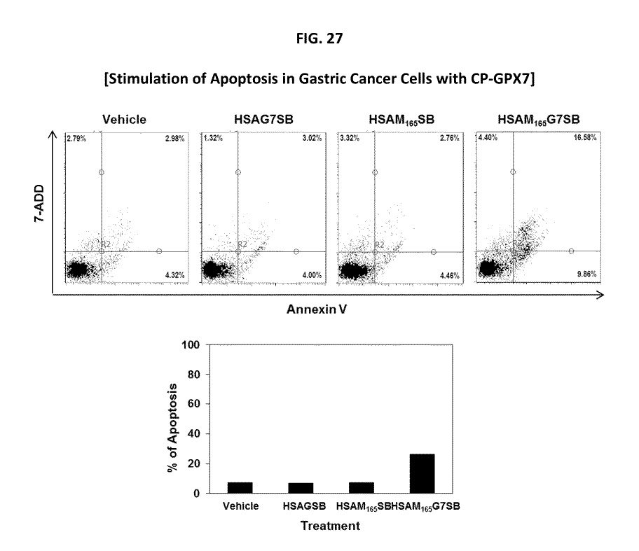

To further determine the effect of CP-GPX7 on the tumorigenicity of GIT-cancer cells, we subsequently investigated whether CP-GPX7 regulates apoptosis in gastric and oesophageal cancer cells. HSAM₁₆₅G7SB protein (CP-GPX7) was a considerably efficient inducer of apoptosis in GIT-cancer cells, as assessed either by a fluorescent terminal dUTP nick-end labeling (TUNEL) assay (FIG. 26) and Annexin V staining (FIG. 27). Consistently, no changes in TUNEL and Annexin V staining were observed in GIT-cancer cells treated with HSAG7SB compared to untreated cell (Vehicle). These results indicate that CP-GPX7 induces apoptosis GIT cancer cells and may suppress the cancer progression by this pathway.

5-5. CP-GPX7 Inhibits Migration of GIT-Cancer Cells

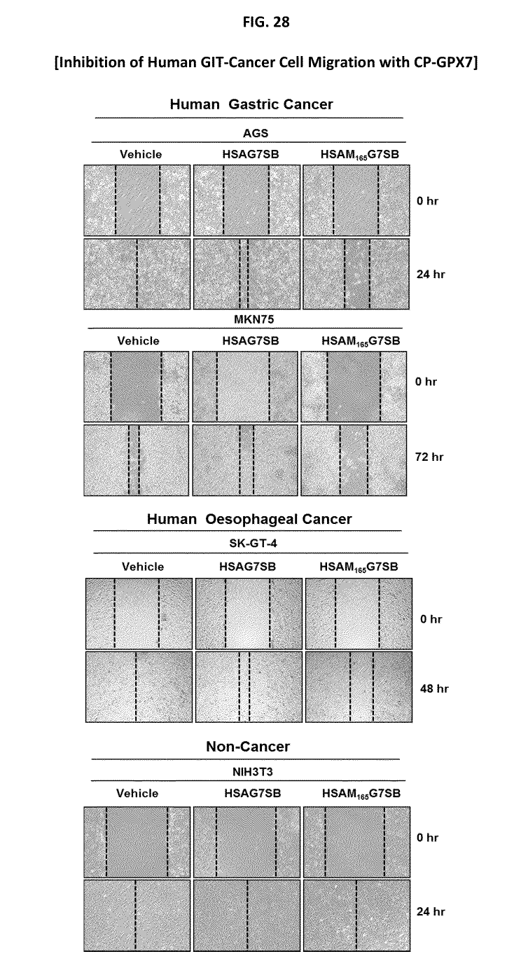

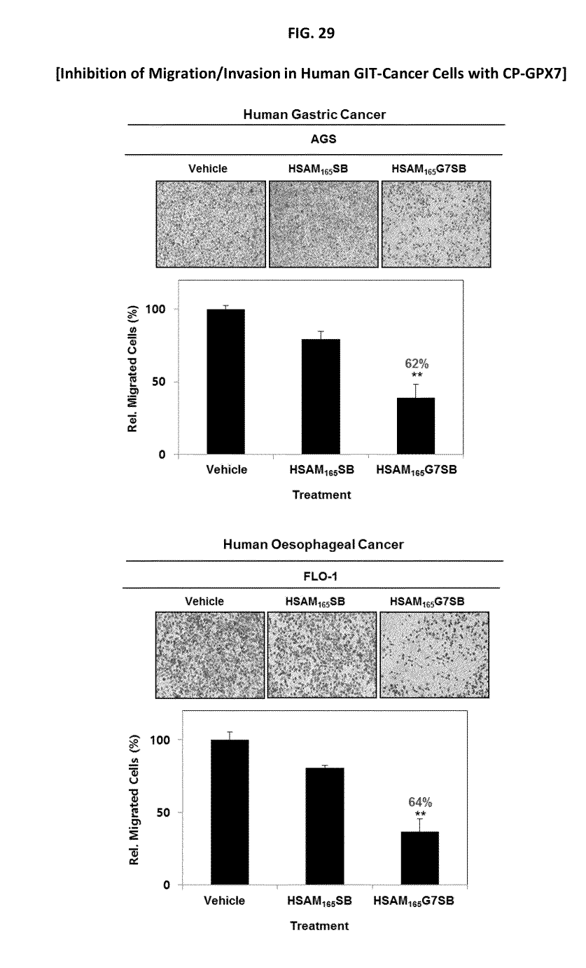

We next determined the ability of CP-GPX7 to influence migration. Human gastric cancer (AGS and MKN75) and oesophageal cancer (SK-GT-4) cells and mouse fibroblast (NIH3T3) were treated with 10 μM of GPX7 recombinant proteins for 2 hours, the monolayers were wounded, and cell migration in the wound was monitored after 24-72 hours (FIG. 28). HSAM₁₆₅G7SB suppressed repopulation of wounded monolayer in cancer cells (AGS, MKN75 and SK-GT-4) by 58%, 47%, and 51%, respectively, although GPX7 recombinant proteins lacking aMTD165 (HSAG7SB) had no effect on the cell migration. Consistent with this, AGS and FLO-1 cells treated with HSAM₁₆₅G7SB recombinant protein also showed significant inhibitory effect on their Transwell migration compared with untreated cells (Vehicle) and non-permeable GPX7 protein-treated cells (HS3B; FIG. 29). Taken together, these data indicate that CP-GPX7 inhibits migratory potential of GIT-cancer cells, suggesting the suppression of metastasis and potentials as an antitumor agent.

### 6. CP-GPX7 Suppresses the Growth of Human Tumor Xenografts

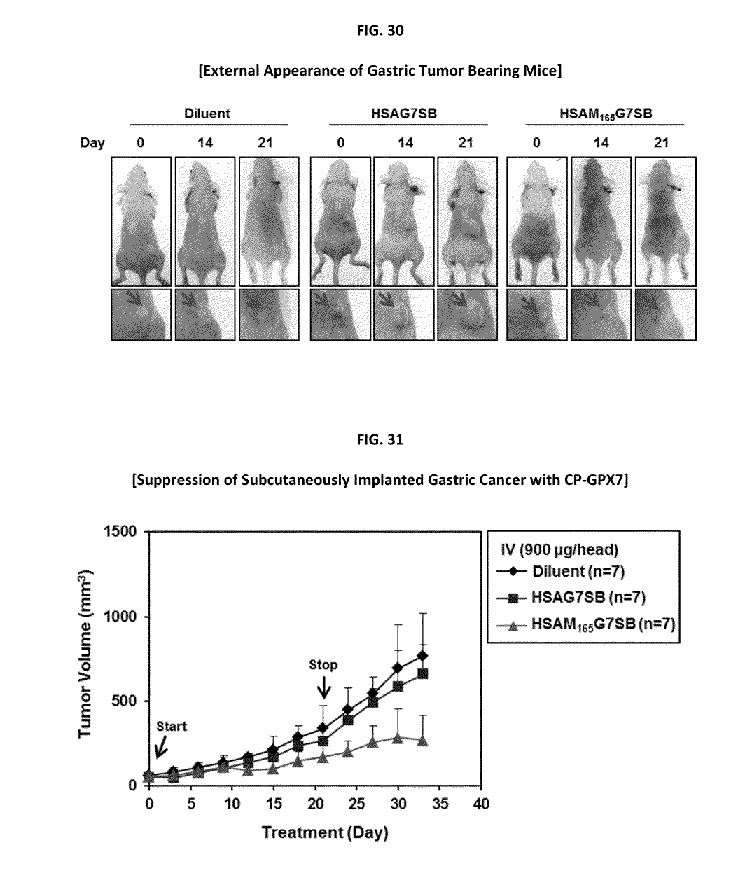

We assessed the anti-tumor activity of CP-GPX7 against human gastric cancer xenografts. Balb/c nu/nu mice were subcutaneously implanted with tumor block (1 mm³) of gastric cancer cells into the left side of the back. Tumor-bearing mice were intravenously administered HSAM₁₆₅G7SB or control proteins (HSAG7SB; 600 μg/head, respectively) for 21 days and observed for 2 weeks following the termination of the treatment (FIG. 30). HSAM₁₆₅G7SB proteins significantly suppressed the tumor growth during the treatment and the effect persisted for at least 2 weeks after the treatment was terminated (56% inhibition at day 21; 70% at day 33, respectively). Whereas, the growth of HSAG7SB-treated tumors increased, matching the rates observed in control mice (Diluent; FIGS. 30 and 31). These results suggest that CP-GPX7 inhibits the growth of established tumors as well as the tumor growth of gastric cancer cells.

### EXAMPLES

The following examples are presented to aid practitioners of the invention, to provide experimental support for the invention, and to provide model protocols. In no way are these examples to be understood to limit the invention.

### Example 1

### Construction of Expression Vectors for GPX7 Recombinant Proteins

His-tagged GPX7 recombinant proteins were designed into the expression vectors which contain human GPX7 proteins fused with aMTD61 or aMTD165 and solubilization domain A (SDA) and solubilization domain B (SDB). The GPX7 sequence was amplified using human genomic DNA as a template. GPX7 and solubilization domains were constructed using primer sets (TABLES 7, 8 and 9) by polymerase chain reaction (PCR).

PCR using 100 ng genomic DNA, 10 pmol each primer, each 0.2 mM dNTP mixture, 1× reaction buffer and 0.5 U Pfu(+) DNA polymerase (MGmed, Seoul, Korea) is following three steps: (i) denaturation (95° C.) for 30 seconds, (ii) annealing (60° C.) for 30 seconds (iii) extension (72° C.) for 1 min each and these steps are repeated 35 times. Set 1 PCR products was cloned at NdeI (5′) and SalI (3′) in pET-28a (+) vector (Novagen, Darmstadt, Germany). Set 2 PCR products was cloned at BamHI (5′) and XhoI (3′) in pET-32a (+) vector (Novagen, Darmstadt, Germany). Set 3 PCR products was cloned at BamHI (5′) and XhoI (3′) in pET-39b (+) vector (Novagen, Darmstadt, Germany) and EcoRI (5′) and XhoI (3′) in pH6HTC His6HaloTag®T7 vector (Promega, Madison, Wis., USA). DNA segments and vectors were cleaved with restriction enzyme (NEB, Hertfordshire, UK) at 37° C. for 3 hours. Digested vectors were ligated with amplified and digested DNA fragments using T4 DNA ligase (NEB, Hertfordshire, UK) at 4° C. overnight. These ligated samples were added 5 μl to competent E. coli DH5-alpha cells (MGmed, Seoul, Korea) on ice for 10 minutes. It moved into the 42° C. water bath for 90 seconds and returned the tubes to ice and incubated for 10 minutes. Then, the mixture added with LB broth media was incubated in 37° C. shaking incubator for 1 hour. The entire culture was plated on LB broth agar plate with kanamycin (25 μg/mL) (Biopure reagents, Seoul, Korea) before incubating overnight at 37° C. Single colony was picked and make a plasmid prep. After the digestion of restriction enzymes, digested plasmid was confirmed by using 1% agarose gels electrophoresis (FIGS. 5, 6, 7 and 8). PCR primers for the His-tagged GPX7 recombinant proteins fused to aMTD and SD are summarized in TABLE 7, 8 and 9. Amino acid sequences of aMTD and SD are shown in SEQ ID NOs: 484, 486, 490, 492, 494, 496, 498 and 500.

### Example 2

### Purification and Preparation of GPX7 Recombinant Proteins

The histidine-tagged GPX7 recombinant proteins were expressed from E. coli BL21-Gold (DE3) competent cells (Agilent Technologies, Santa Clara, USA) grown to an A600 of 0.7 and induced 16 hours at 25° C. with 0.5 mM Isopropyl β-D-1-thiogalactopyranoside (IPTG). The recombinant proteins (HSAM₁₆₅G7SB and HSAM₁₆₅SB) were lysed with lysis buffer A (10 mM imidazole, 50 mM NaH₂PO₄, 300 mM NaCl, pH 8.0), and purified by Ni²⁺ affinity chromatography. Column bound to proteins were washed three times with 30 ml of washing buffer A (20 mM imidazole, 50 mM NaH₂PO₄, 300 mM NaCl, pH 8.0). Ni⁺² affinity purified proteins were eluted three times with 20 mL of elution buffer A (250 mM imidazole, 50 mM NaH₂PO₄, 300 mM NaCl, pH 8.0).

Other recombinant proteins (HG7, HM₆₁G7, HM₆₁G7SA, HM₆₁G7SB, SCHG7M₁₆₅ and HSAG7SB) were expressed from E. coli BL21-Gold (DE3) competent cells grown to an A₆₀₀ of 0.7 and induced 3 hours at 37° C. with 0.7 mM IPTG. Recombinant proteins were lysed using lysis buffer B (8 M Urea, 10 mM Tris, 100 mM NaH₂PO₄) and purified by Ni²⁺ affinity chromatography. Resin bound to proteins were washed 3 times with 30 ml of washing buffer B (8 M Urea, 10 mM Tris, 20 m imidazole, 100 mM NaH₂PO₄). Proteins were eluted 3 times with 30 mL of elution buffer B (8 M Urea, 10 mM Tris, 250 mM imidazole). After purification, they was dialyzed twice against a refolding buffer (550 mM Guanidine-HCl, 440 mM L-Arginine, 50 mM Tris, 100 mM Non-Detergents Sulfobetaines (NDSB), 150 mM NaCl, 2 mM reduced glutathione and 0.2 mM oxidized glutathione). Finally, they were dialyzed against a physiological buffer such as DMEM at 4° C. until the dialysis was over 300×10⁵ times.

After purification, all proteins were dialyzed against DMEM as indicated above. Finally, SDS-PAGE analysis was conducted to confirm the presence of target protein and concentration of purified proteins was quantified using Bradford assay according to the manufacturer's instructions.

Solubility is scored on a 5 point scale ranging from highly soluble proteins with little tendency to precipitate (+++++) to largely insoluble proteins (+) by measuring their turbidity (A450). Yield (mg/L) in physiological buffer condition of each recombinant protein also is determined.

### Example 3

### Determination of Quantitative Cell-Permeability of aMTD/SD-Fused GPX7 Recombinant Proteins

For quantitative cell permeability, the GPX7 recombinant proteins were conjugated to FITC according to the manufacturer's instructions (Pierce Chemical, Rockford, Ill.). RAW 264.7 cells and human gastric cancer cells (AGS and MKN75) were treated with 10 μM FITC-labeled proteins for 1 hour at 37° C., washed with cold PBS three times, treat with proteinase K (10 μg/ml) for 5 minutes at 37° C. to remove cell-surface bound proteins. Cell-permeability of recombinant GPX7 proteins was analyzed by flow cytometry (Guava, Millipore, Darmstadt, Germany).

### Example 4

### Determination of Cell-Permeability and Intracellular Localization of aMTD/SD-Fused GPX7 Recombinant Proteins

For visual cell permeability, NIH3T3 cells were treated with 10 μM FITC-conjugated recombinant proteins for 1 hour at 37° C., washed with cold PBS three times, and treated with proteinase K (10 μg/ml) for 5 minutes at 37° C. Treated cells were fixed in 2% paraformaldehyde (PFA, Junsei, Tokyo, Japan) for 30 minutes at room temperature, washed with cold PBS three times, and mounted with VECTASHIELD Mounting Medium (Vector laboratories, Burlingame, Calif.) with DAPI (4′,6-diamidino 2-phenylindole) for nuclear staining. The intracellular localization of the fluorescent signal is determined by confocal laser scanning microscopy (LM700, Zeiss, Germany).

### Example 5

### Determination of Tissue-Permeability of aMTD/SD-Fused GPX7 Recombinant Proteins

750 μg of FITC-labeled GPX7 recombinant proteins is administered to wild type Balb/c mice. Two hours later, the mice are sacrificed, and liver, kidney, spleen, lung, heart, brain and stomach were isolated. Isolated tissues were washed and frozen with an OCT compound (Sakura, Alphen an den Rijn, Netherlands) on dry ice, and then sectioned to a thickness of 20 μm. The tissue specimens are mounted on a glass slide and observed by fluorescence microscopy (Nikon, Tokyo, Japan).

### Example 6

### Determination of Mechanism of aMTD-Mediated Intracellular Delivery

The GPX7 recombinant proteins were conjugated to FITC according to the manufacturer's instructions (Pierce Chemical, Rockford, Ill.). Human gastric cancer cells (AGS) were pretreated with different agents to assess the effects of various conditions on the protein uptake: (i) 5 μg/ml proteinase K for 5 minutes, (ii) 20 μM Taxol for 30 minutes, (iii) 10 μM antimycin in the presence or absence of 1 mM ATP for 2 hours, (iv) incubation on ice (or maintained at 37° C.) for 15, 30 or 60 minutes, and (v) 100 mM EDTA for 3 hours. Cells were then incubated with 10 μM FITC-labeled GPX7 recombinant proteins for 1 hour at 37° C., washed three times with cold PBS and treated with proteinase K (10 μg/ml) for 10 minutes at 37° C. to remove the cell-surface bound proteins and cell-permeability of GPX7 recombinant proteins was analyzed by flow cytometry (Guava, Millipore, Darmstadt, Germany).

### Example 7

### Determination of Inhibitory Effects of aMTD/SD-Fused GPX7 Recombinant Proteins on GIT-Cancer Cell Viability

Cell viability was evaluated with the CellTiter-Glo luminescent cell viability assay (Promega, Madison, Wis., USA). Human gastric cancer cells (AGS, MKN75, MKN74 and MKN45) and human oesophageal cancer cells (FLO-1, OE19 and OE33) were treated with 10 μM GPX7 recombinant proteins (HSAM₁₆₅G7SB, HSAG7SB and/or HSAM₁₆₅SB) or buffer alone with 2% fetal bovine serum for 72 hours at 37° C. Then, add a volume of CellTiter-Glo reagent equal to the volume of cell culture medium present in each well and incubate for 10 minutes at room temperature, record luminescence using microplate luminometer (LUMIstar Omega, BMG LABTECH, Ortenberg, Germany). The results were relative cell viability compared with the vehicle (buffer alone with 2% FBS)-treated controls.

### Example 8

### Determination of Inhibitory Effects of aMTD/SD-Fused GPX7 Recombinant Proteins on GIT-Cancer Cell Migration

Cancer cell migration was determined using the wound healing assay. Briefly, cells were seeded into 12-well plates and grown to 90% confluence. The wounds were produced by scraping of the cell layer with a sterile white tip. Cells were treated with 10 μM GPX7 recombinant proteins (HSAM₁₆₅G7SB, HSAG7SB and/or HSAM₁₆₅SB) or buffer alone for 2 hours prior to changing the growth medium. Cells were cultured for an additional 24 hours (AGS, SK-GT-4 and NIH3T3) or 72 hours (MKN75) at 37° C. before being photographed. The migration is quantified by counting the number of cells that migrated from the wound edge into the clear area.

### Example 9

Determination of Catalytic Effects of aMTD/SD-Fused GPX7 Recombinant Proteins on GIT-Cancer Cell Apoptosis

Apoptotic cells were analyzed using TUN EL assay with In Situ Cell Death Detection kit TMR red (Roche, 4056 Basel, Switzerland). Human gastric cancer cells (AGS and MKN45) and human oesophageal cancer cells (OE33) were treated with 10 μM GPX7 recombinant proteins (HSAM₁₆₅G7SB and HSAG7SB) or buffer alone for 24 hours with 2% fetal bovine serum, washed with cold PBS two times. Treated cells were fixed in 4% paraformaldehyde (PFA, Junsei, Tokyo, Japan) for 1 hour at room temperature, washed with cold PBS twice and incubated in the permeabilization solution (0.1% Triton X-100 in 0.1% sodium citrate solution) for 2 minutes on the ice. Cells were washed with cold PBS twice, and treated with TUNEL reaction mixture for 1 hour at 37° C. in dark, washed with cold PBS three times and observed by fluorescence microscopy (Nikon, Tokyo, Japan).

### Example 10

### Determination of Mechanism Specific Activity of aMTD/SD-Fused GPX7 Recombinant Proteins in GI-Cancer Cells by Immunofluorescence Assay

Human gastric cancer cells (AGS and MKN45) were grown on glass cover slips to approximately 80% confluency, treated with 10 μM GPX7 recombinant proteins (HSAM₁₆₅G7SB, HSAG7SB and HSAM₁₆₅SB) or buffer alone for 2 hours at 37° C., and washed cold PBS twice. Cells on the coverslips were treated with human TNF-α (50 ng/ml) for 30 minutes, fixed in 4% paraformaldehyde (PFA, Junsei, Tokyo, Japan) for 15 minutes at room temperature, washed cold PBS three times. The coverslips were incubated with 0.1% Triton X-100 (Biopure reagents, Seoul, Korea) for 30 minutes at room temperature, incubated with primary antibody against NF-κB-p65 (1:100, Cell Signaling Technology, Boston, USA) in 5% bovine serum albumin in TBS-T for overnight at 4° C. Secondary antibody was Texas Red®-X goat anti rabbit IgG (1:500, Life technologies, California, USA). The coverslips were mounted on to glass slides using VECTASHIELD Mounting Medium (Vector laboratories, Burlingame, Calif.) with DAPI (4′,6-diamidino 2-phenylindole) for nuclear staining and observed by fluorescence microscopy (Nikon, Tokyo, Japan).

### Example 11

### Determination of Mechanism Specific Activity of aMTD/SD-Fused GPX7 Recombinant Proteins in GI-Cancer Cells by Immunoblotting

Human gastric cancer cells (AGS and MKN74) were cultured in RPMI-1640 medium and maintained at 37° C. in an atmosphere containing 5% CO₂. A total of 5×10⁶ cells were treated with 10 μM GPX7 recombinant proteins (HSAM₁₆₅G7SB and HSAG7SB) or buffer alone for 24 hours. Cells were lysed in a RIPA buffer containing EDTA-free protease inhibitor cocktail (Roche, 4056 Basel, Switzerland) and Halt™ phosphatase inhibitor cocktail (Thermo, Rockford, USA). Equal amounts of cell lysate protein were subjected to SDS-PAGE and transferred to nitrocellulose membranes. The protein transferred membranes were incubated to block non-specific binding sites in immersing the membrane in 5% non-fat dried milk. The membranes were incubated with the rabbit anti-phospho RB antibody, the rabbit anti-phospho NF-κB p65 antibody, the rabbit anti-p21 antibody and the rabbit anti-β-actin antibody diluted 1:1000 in 5% bovine serum albumin in TBS-T at 4° C. for 24 hours. Secondary antibody was anti-rabbit IgG-HRP diluted 1:5000 in 5% skim milk in TBS-T for 1 hour at room temperature. All antibodies were purchased from Cell Signaling Technology (Boston, USA). The blots were developed using a chemiluminescence detection system, SuperSignal® West Dura Extended Duration Substrate (Thermo, Rockford, USA) and exposed to an x-ray film.

It will be apparent to those skilled in the art that various modifications can be made to the above-described exemplary embodiments of the present invention without departing from the spirit or scope of the invention. Thus, it is intended that the present invention covers all such modifications provided that they come within the scope of the appended claims and their equivalents.

[cDNA Sequence of Histidine Tag]
SEQ ID NO: 481
ATGGGCAGCAGCCATCATCATCATCATCACAGCAGCGGCCTGGTGCCGCGCGGCAGC
[Amino Acid Sequence of Histidine Tag]
SEQ ID NO: 482
Met Gly Ser Ser His His His His His His Ser Ser Gly Leu Val Pro Arg Gly Ser
[cDNA Sequences of aMTD61]
SEQ ID NO: 483
GTGGCGGCGCTGCCGGTGCTGCTGGCGGCGCTGCCG
[Amino Acid Sequences of aMTD61]
SEQ ID NO: 484
Please see TABLE 4
[cDNA Sequences of aMTD165]
SEQ ID NO: 485
CCGGTGGCGGTGCCGGTGGCGCTGGCGATTGTGCCG
[Amino Acid Sequences of aMTD165]
SEQ ID NO: 486
Please see TABLE 4
[cDNA Sequence of human GPX7]
SEQ ID NO: 487
ATGGTGGCGG CGACGGTGGC AGCGGCGTGG CTGCTCCTGT GGGCTGCGGC CTGCGCGCAG CAGGAGCAGG
ACTTCTACGA CTTCAAGGCG GTCAACATCC GGGGCAAACT GGTGTCGCTG GAGAAGTACC GCGGATCGGT
GTCCCTGGTG GTGAATGTGG CCAGCGAGTG CGGCTTCACA GACCAGCACT ACCGAGCCCT GCAGCAGCTG
CAGCGAGACC TGGGCCCCCA CCACTTCAAC GTGCTCGCCT TCCCCTGCAA CCAGTTTGGC CAACAGGAGC
CTGACAGCAA CAAGGAGATT GAGAGCTTTG CCCGCCGCAC CTACAGTGTC TCATTCCCCA TGTTTAGCAA
GATTGCAGTC ACCGGTACTG GTGCCCATCC TGCCTTCAAG TACCTGGCCC AGACTTCTGG GAAGGAGCCC
ACCTGGAACT TCTGGAAGTA CCTAGTAGCC CCAGATGGAA AGGTGGTAGG GGCTTGGGAC CCAACTGTGT
CAGTGGAGGA GGTCAGACCC CAGATCACAG CGCTCGTGAG GAAGCTCATC CTACTGAAGC GAGAAGACTT
A
[Amino Acid Sequence of human GPX7]
SEQ ID NO: 488
Met Val Ala Ala Thr Val Ala Ala Ala Trp Leu Leu Leu Trp Ala Ala Ala Cys Ala
Gln Gln Glu Gln Asp Phe Tyr Asp Phe Lys Ala Val Asn Ile Arg Gly Lys Leu Val
Ser Leu Glu Lys Tyr Arg Gly Ser Val Ser Leu Val Val Asn Val Ala Ser Glu Cys
Gly Phe Thr Asp Gln His Tyr Arg Ala Leu Gln Gln Leu Gln Arg Asp Leu Gly Pro
His His Phe Asn Val Leu Ala Phe Pro Cys Asn Gln Phe Gly Gln Gln Glu Pro Asp
Ser Asn Lys Glu Ile Glu Ser Phe Ala Arg Arg Thr Tyr Ser Val Ser Phe Pro Met
Phe Ser Lys Ile Ala Val Thr Gly Thr Gly Ala His Pro Ala Phe Lys Tyr Leu Ala
Gln Thr Ser Gly Lys Glu Pro Thr Trp Asn Phe Trp Lys Tyr Leu Val Ala Pro Asp
Gly Lys Val Val Gly Ala Trp Asp Pro Thr Val Ser Val Glu Glu Val Arg Pro Gln
Ile Thr Ala Leu Val Arg Lys Leu Ile Leu Leu Lys Arg Glu Asp Leu
[cDNA Sequences of SDA]
SEQ ID NO: 489
ATGGCAAATA TTACCGTTTT CTATAACGAA GACTTCCAGG GTAAGCAGGT CGATCTGCCG CCTGGCAACT
ATACCCGCGC CCAGTTGGCG GCGCTGGGCA TCGAGAATAA TACCATCAGC TCGGTGAAGG TGCCGCCTGG
CGTGAAGGCT ATCCTGTACC AGAACGATGG TTTCGCCGGC GACCAGATCG AAGTGGTGGC CAATGCCGAG
GAGTTGGGCC CGCTGAATAA TAACGTCTCC AGCATCCGCG TCATCTCCGT GCCCGTGCAG CCGCGCATGG
CAAATATTAC CGTTTTCTAT AACGAAGACT TCCAGGGTAA GCAGGTCGAT CTGCCGCCTG GCAACTATAC
CCGCGCCCAG TTGGCGGCGC TGGGCATCGA GAATAATACC ATCAGCTCGG TGAAGGTGCC GCCTGGCGTG
AAGGCTATCC TCTACCAGAA CGATGGTTTC GCCGGCGACC AGATCGAAGT GGTGGCCAAT GCCGAGGAGC
TGGGTCCGCT GAATAATAAC GTCTCCAGCA TCCGCGTCAT CTCCGTGCCG GTGCAGCCGA GG
[Amino Acid Sequences of SDA]
SEQ ID NO: 490
Met Ala Asn Ile Thr Val Phe Tyr Asn Glu Asp Phe Gln Gly Lys Gln Val Asp Leu
Pro Pro Gly Asn Tyr Thr Arg Ala Gln Leu Ala Ala Leu Gly Ile Glu Asn Asn Thr
Ile Ser Ser Val Lys Val Pro Pro Gly Val Lys Ala Ile Leu Tyr Gln Asn Asp Gly
Phe Ala Gly Asp Gln Ile Glu Val Val Ala Asn Ala Glu Glu Leu Gly Pro Leu Asn
Asn Asn Val Ser Ser Ile Arg Val Ile Ser Val Pro Val Gln Pro Arg Met Ala Asn
Ile Thr Val Phe Tyr Asn Glu Asp Phe Gln Gly Lys Gln Val Asp Leu Pro Pro Gly
Asn Tyr Thr Arg Ala Gln Leu Ala Ala Leu Gly Ile Glu Asn Asn Thr Ile Ser Ser
Val Lys Val Pro Pro Gly Val Lys Ala Ile Leu Tyr Gln Asn Asp Gly Phe Ala Gly
Asp Gln Ile Glu Val Val Ala Asn Ala Glu Glu Leu Gly Pro Leu Asn Asn Asn Val
Ser Ser Ile Arg Val Ile Ser Val Pro Val Gln Pro Arg
[cDNA Sequences of SDB]
SEQ ID NO: 491
ATGGCAGAAC AAAGCGACAA GGATGTGAAG TACTACACTC TGGAGGAGAT TCAGAAGCAC AAAGACAGCA
AGAGCACCTG GGTGATCCTA CATCATAAGG TGTACGATCT GACCAAGTTT CTCGAAGAGC ATCCTGGTGG
GGAAGAAGTC CTGGGCGAGC AAGCTGGGGG TGATGCTACT GAGAACTTTG AGGACGTCGG GCACTCTACG
GATGCACGAG AACTGTCCAA AACATACATC ATCGGGGAGC TCCATCCAGA TGACAGATCA AAGATAGCCA
AGCCTTCGGA AACCCTT
[Amino Acid Sequences of SDB]
SEQ ID NO: 492
Met Ala Glu Gln Ser Asp Lys Asp Val Lys Tyr Tyr Thr Leu Glu Glu Ile Gln Lys
His Lys Asp Ser Lys Ser Thr Trp Val Ile Leu His His Lys Val Tyr Asp Leu Thr
Lys Phe Leu Glu Glu His Pro Gly Gly Glu Glu Val Leu Gly Glu Gln Ala Gly Gly
Asp Ala Thr Glu Asn Phe Glu Asp Val Gly His Ser Thr Asp Ala Arg Glu Leu Ser
Lys Thr Tyr Ile Ile Gly Glu Leu His Pro Asp Asp Arg Ser Lys Ile Ala Lys Pro
Ser Glu Thr Leu
[cDNA Sequences of SDC]
SEQ ID NO: 493
ATGAGCGATA AAATTATTCA CCTGACTGAC GACAGTTTTG ACACGGATGT ACTCAAAGCG GACGGGGCGA
TCCTCGTCGA TTTCTGGGCA GAGTGGTGCG GTCCGTGCAA AATGATCGCC CCGATTCTGG ATGAAATCGC
TGACGAATAT CAGGGCAAAC TGACCGTTGC AAAACTGAAC ATCGATCAAA ACCCTGGCAC TGCGCCGAAA
TATGGCATCC GTGGTATCCC GACTCTGCTG CTGTTCAAAA ACGGTGAAGT GGCGGCAACC AAAGTGGGTG
CACTGTCTAA AGGTCAGTTG AAAGAGTTCC TCGACGCTAA CCTGGCC
[Amino Acid Sequences of SDC]
SEQ ID NO: 494
Met Ser Asp Lys Ile Ile His Leu Thr Asp Asp Ser Phe Asp Thr Asp Val Leu Lys
Ala Asp Gly Ala Ile Leu Val Asp Phe Trp Ala Glu Trp Cys Gly Pro Cys Lys Met
Ile Ala Pro Ile Leu Asp Glu Ile Ala Asp Glu Tyr Gln Gly Lys Leu Thr Val Ala
Lys Leu Asn Ile Asp Gln Asn Pro Gly Thr Ala Pro Lys Tyr Gly Ile Arg Gly Ile
Pro Thr Leu Leu Leu Phe Lys Asn Gly Glu Val Ala Ala Thr Lys Val Gly Ala Leu
Ser Lys Gly Gln Leu Lys Glu Phe Leu Asp Ala Asn Leu Ala
[cDNA Sequences of SDD]
SEQ ID NO: 495
ATGAAAAAGA TTTGGCTGGC GCTGGCTGGT TTAGTTTTAG CGTTTAGCGC ATCGGCGGCG CAGTATGAAG
ATGGTAAACA GTACACTACC CTGGAAAAAC CGGTAGCTGG CGCGCCGCAA GTGCTGGAGT TTTTCTCTTT
CTTCTGCCCG CACTGCTATC AGTTTGAAGA AGTTCTGCAT ATTTCTGATA ATGTGAAGAA AAAACTGCCG
GAAGGCGTGA AGATGACTAA ATACCACGTC AACTTCATGG GTGGTGACCT GGGCAAAGAT CTGACTCAGG
CATGGGCTGT GGCGATGGCG CTGGGCGTGG AAGACAAAGT GACTGTTCCG CTGTTTGAAG GCGTACAGAA
AACCCAGACC ATTCGTTCTG CTTCTGATAT CCGCGATGTA TTTATCAACG CAGGTATTAA AGGTGAAGAG
TACGACGCGG CGTGGAACAG CTTCGTGGTG AAATCTCTGG TCGCTCAGCA GGAAAAAGCT GCAGCTGACG
TGCAATTGCG TGGCGTTCCG GCGATGTTTG TTAACGGTAA ATATCAGCTG AATCCGCAGG GTATGGATAC
CAGCAATATG GATGTTTTTG TTCAGCAGTA TGCTGATACA GTGAAATATC TGTCCGAGAA AAAA
[Amino Acid Sequences of SDD]
SEQ ID NO: 496
Met Lys Lys Ile Trp Leu Ala Leu Ala Gly Leu Val Leu Ala Phe Ser Ala Ser Ala
Ala Gln Tyr Glu Asp Gly Lys Gln Tyr Thr Thr Leu Glu Lys Pro Val Ala Gly Ala
Pro Gln Val Leu Glu Phe Phe Ser Phe Phe Cys Pro His Cys Tyr Gln Phe Glu Glu
Val Leu His Ile Ser Asp Asn Val Lys Lys Lys Leu Pro Glu Gly Val Lys Met Thr
Lys Tyr His Val Asn Phe Met Gly Gly Asp Leu Gly Lys Asp Leu Thr Gln Ala Trp
Ala Val Ala Met Ala Leu Gly Val Glu Asp Lys Val Thr Val Pro Leu Phe Glu Gly
Val Gln Lys Thr Gln Thr Ile Arg Ser Ala Ser Asp Ile Arg Asp Val Phe Ile Asn
Ala Gly Ile Lys Gly Glu Glu Tyr Asp Ala Ala Trp Asn Ser Phe Val Val Lys Ser
Leu Val Ala Gln Gln Glu Lys Ala Ala Ala Asp Val Gln Leu Arg Gly Val Pro Ala
Met Phe Val Asn Gly Lys Tyr Gln Leu Asn Pro Gln Gly Met Asp Thr Ser Asn Met
Asp Val Phe Val Gln Gln Tyr Ala Asp Thr Val Lys Tyr Leu Ser Glu Lys Lys
[cDNA Sequences of SDE]
SEQ ID NO: 497
GGGTCCCTGC AGGACTCAGA AGTCAATCAA GAAGCTAAGC CAGAGGTCAA GCCAGAAGTC AAGCCTGAGA
CTCACATCAA TTTAAAGGTG TCCGATGGAT CTTCAGAGAT CTTCTTCAAG ATCAAAAAGA CCACTCCTTT
AAGAAGGCTG ATGGAAGCGT TCGCTAAAAG ACAGGGTAAG GAAATGGACT CCTTAACGTT CTTGTACGAC
GGTATTGAAA TTCAAGCTGA TCAGACCCCT GAAGATTTGG ACATGGAGGA TAACGATATT ATTGAGGCTC
ACCGCGAACA GATTGGAGGT
[Amino Acid Sequences of SDE]
SEQ ID NO: 498
Gly Ser Leu Gln Asp Ser Glu Val Asn Gln Glu Ala Lys Pro Glu Val Lys Pro Glu
Val Lys Pro Glu Thr His Ile Asn Leu Lys Val Ser Asp Gly Ser Ser Glu Ile Phe
Phe Lys Ile Lys Lys Thr Thr Pro Leu Arg Arg Leu Met Glu Ala Phe Ala Lys Arg
Gln Gly Lys Glu Met Asp Ser Leu Thr Phe Leu Tyr Asp Gly Ile Glu Ile Gln Ala
Asp Gln Thr Pro Glu Asp Leu Asp Met Glu Asp Asn Asp Ile Ile Glu Ala His Arg
Glu Gln Ile Gly Gly
[cDNA Sequences of SDF]
SEQ ID NO: 499
GGATCCGAAA TCGGTACTGG CTTTCCATTC GACCCCCATT ATGTGGAAGT CCTGGGCGAG CGCATGCACT
ACGTCGATGT TGGTCCGCGC GATGGCACCC CTGTGCTGTT CCTGCACGGT AACCCGACCT CCTCCTACGT
GTGGCGCAAC ATCATCCCGC ATGTTGCACC GACCCATCGC TGCATTGCTC CAGACCTGAT CGGTATGGGC
AAATCCGACA AACCAGACCT GGGTTATTTC TTCGACGACC ACGTCCGCTT CATGGATGCC TTCATCGAAG
CCCTGGGTCT GGAAGAGGTC GTCCTGGTCA TTCACGACTG GGGCTCCGCT CTGGGTTTCC ACTGGGCCAA
GCGCAATCCA GAGCGCGTCA AAGGTATTGC ATTTATGGAG TTCATCCGCC CTATCCCGAC CTGGGACGAA
TGGCCAGAAT TTGCCCGCGA GACCTTCCAG GCCTTCCGCA CCACCGACGT CGGCCGCAAG CTGATCATCG
ATCAGAACGT TTTTATCGAG GGTACGCTGC CGATGGGTGT CGTCCGCCCG CTGACTGAAG TCGAGATGGA
CCATTACCGC GAGCCGTTCC TGAATCCTGT TGACCGCGAG CCACTGTGGC GCTTCCCAAA CGAGCTGCCA
ATCGCCGGTG AGCCAGCGAA CATCGTCGCG CTGGTCGAAG AATACATGGA CTGGCTGCAC CAGTCCCCTG
TCCCGAAGCT GCTGTTCTGG GGCACCCCAG GCGTTCTGAT CCCACCGGCC GAAGCCGCTC GCCTGGCCAA
AAGCCTGCCT AACTGCAAGG CTGTGGACAT CGGCCCGGGT CTGAATCTGC TGCAAGAAGA CAACCCGGAC
CTGATCGGCA GCGAGATCGC GCGCTGGCTG TCTACTCTGG AGATTTCCGG T
[Amino Acid Sequences of SDF]
SEQ ID NO: 500
Gly Ser Glu Ile Gly Thr Gly Phe Pro Phe Asp Pro His Tyr Val Glu Val Leu Gly
Glu Arg Met His Tyr Val Asp Val Gly Pro Arg Asp Gly Thr Pro Val Leu Phe Leu
His Gly Asn Pro Thr Ser Ser Tyr Val Trp Arg Asn Ile Ile Pro His Val Ala Pro
Thr His Arg Cys Ile Ala Pro Asp Leu Ile Gly Met Gly Lys Ser Asp Lys Pro Asp
Leu Gly Tyr Phe Phe Asp Asp His Val Arg Phe Met Asp Ala Phe Ile Glu Ala Leu
Gly Leu Glu Glu Val Val Leu Val Ile His Asp Trp Gly Ser Ala Leu Gly Phe His
Trp Ala Lys Arg Asn Pro Glu Arg Val Lys Gly Ile Ala Phe Met Glu Phe Ile Arg
Pro Ile Pro Thr Trp Asp Glu Trp Pro Glu Phe Ala Arg Glu Thr Phe Gln Ala Phe
Arg Thr Thr Asp Val Gly Arg Lys Leu Ile Ile Asp Gln Asn Val Phe Ile Glu Gly
Thr Leu Pro Met Gly Val Val Arg Pro Leu Thr Glu Val Glu Met Asp His Tyr Arg
Glu Pro Phe Leu Asn Pro Val Asp Arg Glu Pro Leu Trp Arg Phe Pro Asn Glu Leu
Pro Ile Ala Gly Glu Pro Ala Asn Ile Val Ala Leu Val Glu Glu Tyr Met Asp Trp
Leu His Gln Ser Pro Val Pro Lys Leu Leu Phe Trp Gly Thr Pro Gly Val Leu Ile
Pro Pro Ala Glu Ala Ala Arg Leu Ala Lys Ser Leu Pro Asn Cys Lys Ala Val Asp
Ile Gly Pro Gly Leu Asn Leu Leu Gln Glu Asp Asn Pro Asp Leu Ile Gly Ser Glu
Ile Ala Arg Trp Leu Ser Thr Leu Glu Ile Ser Gly

What is claimed is:

1. GPX7 recombinant proteins fused to newly invented hydrophobic cell-penetrating peptides (CPPs)—advanced macromolecule transduction domains (aMTDs) and solubilization domain (SD)

2. The GPX7 recombinant proteins according to claim 1, wherein aMTDs are selected from the group consisting of SEQ ID NO: 1 to SEQ ID NO: 240.

3. The GPX7 recombinant proteins according to claim 1, wherein SDs are selected from the group consisting of SEQ ID NO: 490, SEQ ID NO: 492, SEQ ID NO: 494, SEQ ID NO: 496, SEQ ID NO: 498, SEQ ID NO: 500.

4. The GPX7 recombinant proteins according to claim 1, wherein SDs are fused to GPX7 recombinant proteins for high solubility and yield.

5. Isolated polynucleotides that encode GPX7 recombinant proteins according to claim 1.

6. The isolated polynucleotides according to claim 5, wherein the isolated polynucleotide of aMTDs are selected from the group consisting of SEQ ID NO: 241 to SEQ ID NO: 480.

7. The isolated polynucleotides according to claim 5, wherein the isolated polynucleotide of SDs are selected from the group consisting of SEQ ID NO: 489, SEQ ID NO: 491, SEQ ID NO: 493, SEQ ID NO: 495, SEQ ID NO: 497, SEQ ID NO: 499.

8. The result of therapeutic applicability in gastrointestinal track (GIT)-specific cancer with GPX7 recombinant proteins fused to newly invented hydrophobic cell-penetrating peptides (CPPs), namely advanced macromolecule transduction domains (aMTDs) and solubilization domain (SD)
